(19) United States
(12) Patent Application Publication
(10) Pub. No.: US 2010/0093733 A1
(43) Pub. Date: Apr. 15, 2010

(54) AMIDE AND UREA DERIVATIVES FOR THE TREATMENT OF METABOLIC DISEASES

(75) Inventors: Oscar Barba, Oxfordshire (GB); Graham Dawson, Oxfordshire (GB); William Gattrell, Oxfordshire (GB); Martin James Procter, Oxfordshire (GB); Chrystelle Marie Rasamison, Oxfordshire (GB); Colin Peter Sambrook-Smith, Oxfordshire (GB); Philippe Wong-Kai-In, Oxfordshire (GB)

Correspondence Address: OSI PHARMACEUTICALS, INC., 41 PINELAWN ROAD, MELVILLE, NY, 11747, US

(73) Assignee: PROSIDION LIMITED, Oxford, Oxfordshire (GB)

(21) Appl. No.: 12/527,162

(22) PCT Filed: Feb. 15, 2008
(86) PCT No.: PCT/GB08/50103

§ 371 Date: Dec. 4, 2009

(30) Foreign Application Priority Data
Feb. 15, 2007 (GB) 0702960.6
Feb. 15, 2007 (GB) 0702961.4

(51) Int. Cl. A61K 31/535; C07D 213/56; A61K 31/44; C07D 277/00; A61K 31/425; C07D 231/00; A61K 31/415; C07D 295/12; C07C 229/00; A61K 31/24; C07C 233/00; A61K 31/165; C07D 217/00; A61K 31/47; C07D 471/02; C07D 261/20; A61K 31/42; C07D 235/00; C07D 409/00; C07D 317/44; A61K 31/36; C07D 261/06; C07D 401/00; C07D 237/28; A61K 31/495; C07D 207/00; A61K 31/40; A61K 31/335

(52) U.S. Cl. 514/237.5; 546/316; 514/355; 548/200; 514/365; 548/374.1; 514/406; 544/165; 560/43; 514/539; 564/184; 514/617; 546/146; 514/307; 546/115; 514/302; 548/241; 514/379; 548/309.4; 514/393; 546/281.1; 514/337; 549/441; 514/464; 548/247; 514/378; 546/268.1; 514/300; 544/235; 514/249; 548/533; 514/423; 549/436; 514/452

(57) ABSTRACT

Compounds of formula (I): or pharmaceutically acceptable salts and esters thereof, are useful for the treatment of obesity, type II diabetes and the metabolic syndrome.

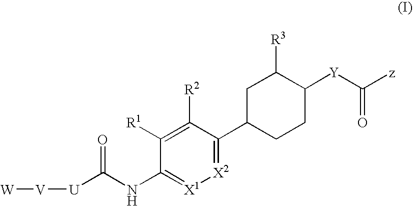

### BACKGROUND OF THE INVENTION

The invention relates to inhibitors of diacylglycerol acyltransferase. The inhibitors are amide and urea derivatives which are useful for the treatment of diseases such as obesity, type II diabetes mellitus and metabolic syndrome.

Obesity is characterized by an excessive adipose tissue mass relative to body size. Clinically, body fat mass is estimated by the body mass index (BMI; weight(kg)/height(m)²), or waist circumference. Individuals are considered obese when the BMI is greater than 30 and there are established medical consequences of being overweight. It has been an accepted medical view for some time that an increased body weight, especially as a result of abdominal body fat, is associated with an increased risk for diabetes, hypertension, heart disease, and numerous other health complications, such as arthritis, stroke, gallbladder disease, muscular and respiratory problems, back pain and even certain cancers.

Pharmacological approaches to the treatment of obesity have been mainly concerned with reducing fat mass by altering the balance between energy intake and expenditure. Many studies have clearly established the link between adiposity and the brain circuitry involved in the regulation of energy homeostasis. Direct and indirect evidence suggest that serotonergic, dopaminergic, adrenergic, cholinergic, endocannabinoid, opioid, and histaminergic pathways in addition to many neuropeptide pathways (e.g. neuropeptide Y and melanocortins) are implicated in the central control of energy intake and expenditure. Hypothalamic centres are also able to sense peripheral hormones involved in the maintenance of body weight and degree of adiposity, such as insulin and leptin, and fat tissue derived peptides.

Drugs aimed at the pathophysiology associated with insulin dependent Type I diabetes and non-insulin dependent Type II diabetes have many potential side effects and do not adequately address the dyslipidaemia and hyperglycaemia in a high proportion of patients. Treatment is often focused at individual patient needs using diet, exercise, hypoglycaemic agents and insulin, but there is a continuing need for novel antidiabetic agents, particularly ones that may be better tolerated with fewer adverse effects.

Similarly, metabolic syndrome (syndrome X) which is characterized by hypertension and its associated pathologies including atherosclerosis, lipidemia, hyperlipidemia and hypercholesterolemia have been associated with decreased insulin sensitivity which can lead to abnormal blood sugar levels when challenged. Myocardial ischemia and microvascular disease is an established morbidity associated with untreated or poorly controlled metabolic syndrome.

There is a continuing need for novel antiobesity and antidiabetic agents, particularly ones that are well tolerated with few adverse effects.

Disorders or imbalances in triglyceride metabolism have been implicated in the pathogenesis of a variety of disease risks, including obesity, insulin resistance syndrome, type II diabetes, metabolic syndrome (syndrome X) and coronary heart disease.

Diacylglycerol O-acyltransferase (DGAT) is a key enzyme in triglyceride synthesis. DGAT catalyzes the final and rate limiting step in triacylglycerol synthesis from 1,2-diacylglycerol (DAG) and long chain fatty acyl CoA as substrates. Thus, DGAT plays an essential role in the metabolism of cellular diacylglycerol and is important for triglyceride production and energy storage homeostasis (Mayorek et al, European Journal of Biochemistry (1989) 182, 395-400).

Two forms of DGAT have been cloned: DGAT1 and DGAT2 (Cases et al, Proceedings of the National Academy of Science, USA (1998) 95, 13018-13023, Lardizabal et al, Journal of Biological Chemistry (2001) 276, 38862-38869 and Cases et al, Journal of Biological Chemistry (2001) 276, 38870-38876). Although both enzymes utilize the same substrates, there is no significant homology between DGAT1 and DGAT2. Further, although both enzymes are widely expressed, differences exist in the relative abundance of DGAT1 and DGAT2 expression in various tissues.

DGAT1 knock-out mice do not become obese when challenged with a high fat diet in contrast to wild-type littermates (Smith et al., Nature Genetics 25:87-90, 2000). DGAT1 knock-out mice display reduced postprandial plasma glucose levels and exhibit increased energy expenditure, but have normal levels of serum triglycerides (Smith et al., 2000), possibly due to the preserved DGAT2 activity. Since DGAT1 is expressed in the intestine and adipose tissue (Cases et al., 1998), there are at least two possible mechanisms to explain the resistance of DGAT1 knock-out mice to diet induced obesity. First, abolishing DGAT1 activity in the intestine may block the reformation and export of triacylglycerol from intestinal cells into the circulation via chylomicron particles. Second, knocking out DGAT1 activity in the adipocyte may decrease deposition of triacylglycerol in the white adipose tissue.

Compounds that decrease the synthesis of triglycerides from diacylglycerol by inhibiting or lowering the activity of the DGAT1 enzyme are predicted to be of value as therapeutic agents for the treatment diseases associated with abnormal metabolism of triglycerides. Thus a need exits for additional DGAT1 inhibitors that have efficacy for the treatment of metabolic disorders such as, for example, obesity, type II diabetes mellitus and metabolic syndrome.

### SUMMARY OF THE INVENTION

Compounds of formula (I)

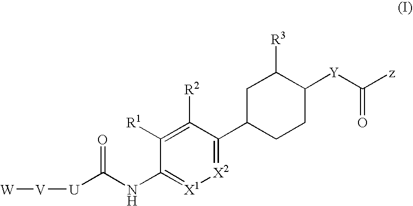

one of X¹ and X² is N and the other is CR⁴;

R¹, R² and R⁴ independently represent a group selected from hydrogen, F, Cl, methyl and methoxy;

R³ represents hydrogen, C₁-C₃ alkyl, hydroxy or C₁-C₃ alkoxy;

Y represents a bond, or —(CH₂)ₙ(CR¹⁰R¹³)ₒ(CH₂)ₚ—; or when R³ represents hydrogen or C₁-C₃ alkyl then Y may also represent —O—(CH₂)ₙ(CR¹⁰R¹³)ₒ(CH₂)ₚ—;

n represents an integer 0 to 3;

o represents 0 or 1;

p represents an integer 0 to 3;

provided that n+o+p is 1, 2 or 3;

Z represents hydroxy, C₁-C₆ alkoxy or —NR⁶R⁷;

R⁶ represents hydrogen, C₁-C₆ alkyl or C₃-C₆ cycloalkyl either of which groups may optionally be substituted by one or two groups selected from hydroxy, C₁-C₃ alkoxy, —NR⁸R⁹, phenyl, —CONR¹¹R¹² and COOR⁵, provided that there are at least two carbon atoms between the nitrogen of the —NR⁶R⁷ group and any hydroxy, C₁-C₃ alkoxy, or —NR⁸R⁹ substituent on R⁶;

R⁷ represents hydrogen or C₁-C₆ alkyl;

or R⁶ and R⁷ are joined such that —NR⁶R⁷ forms a 5- to 7-membered heterocyclic ring optionally containing an additional heteroatom selected from N, O and S, which ring may optionally be substituted by a group selected from hydroxy, C₁-C₃ alkoxy, C₁-C₃ alkyl, CH₂OH, CH₂OMe and COOH;

U represents a bond or >N-Q;

Q represents hydrogen, or Q is joined to W such that WVNQ together form a 5- to 7-membered nitrogen containing heterocyclic ring fused to a phenyl which may optionally be substituted as for W;

when U represents a bond, V represents a bond, (CH₂)ₘ which may be optionally substituted by C₁-C₃ alkyl, hydroxy or C₁-C₃ alkoxy, —(CH₂)ₐO(CH₂)_{b}—, —(CH₂)_{c}OCH(Me)—, —NR¹⁴(CH₂)_{d}— or cyclopropyl in which the points of attachment are 1,1 or 1,2;

when U represents >N-Q, V represents a bond or (CH₂)ₘ;

m represents an integer 1 to 3;

a represents 0 or 1;

b represents 1 or 2;

c represents 0 or 1;

d represents 1 or 2;

R⁵, R⁸, R⁹, R¹¹, R¹² and R¹⁴ independently represent hydrogen or C₁-C₃ alkyl;

R¹⁰ and R¹³ independently represent hydrogen or C₁-C₃ alkyl or, together with the carbon atom to which they are attached, can be joined to form a C₃-C₆ cycloalkyl ring; and

W represents a 5- to 10-membered monocyclic or bicyclic aromatic or heteroaromatic ring, which bicyclic rings may contain one unsaturated ring; any of said rings being optionally substituted by one or more groups selected from halo, hydroxy, cyano, C₁-C₆ alkyl or C₁-C₃ alkoxy, either of which may be substituted by one or more fluoro atoms; and when W is monocyclic may also be optionally substituted by phenyl, a 5- to 6-membered heteroaromatic, 5- to 7-membered heterocyclic or C₃-C₆ cycloalkyl ring;

or a pharmaceutically acceptable salt or ester thereof.

### DETAILED DESCRIPTION OF THE INVENTION

In one embodiment of the invention U represents a bond. In a further embodiment U represents >N-Q.

In one embodiment of the invention X¹ represents CR⁴. In a further embodiment X² represents CR⁴.

In one embodiment of the invention Y represents a bond. In another embodiment of the invention Y represents —(CH₂)ₙ(CR¹⁰R¹³)ₒ(CH₂)ₚ—, for example (CH₂)ₙ. In another embodiment of the invention Y represents —O—(CH₂)ₙ(CR¹⁰R¹³)ₒ(CH₂)ₚ—, for example —O—(CH₂)ₙ—.

Preferred compounds of the invention include those wherein:

R¹ represents hydrogen, F, Cl or Me, for example hydrogen, F or Cl, especially hydrogen.

R² represents hydrogen, F, Cl or Me, for example hydrogen, Cl or Me, especially hydrogen.

R³ represents hydrogen, hydroxy or OMe, for example hydrogen or hydroxy, especially hydrogen.

X¹ represents CR⁴.

R⁴ represents hydrogen.

R⁵ represents hydrogen.

n represents 1 or 2, particularly 1.

o represents 0.

p represents 0.

Y represents a bond or CH₂, particularly a bond.

In one embodiment of the invention Z represents hydroxy. In a specific embodiment of the invention Y represents CH₂ and Z represents hydroxy. In another specific embodiment of the invention Y represents CH₂CH₂ and Z represents hydroxy. In another specific embodiment of the invention Y represents a bond and Z represents hydroxy.

In another embodiment of the invention Z represents —NR⁶R⁷.

In another embodiment of the invention Z represents C₁-C₆ alkoxy.

When R⁶ and R⁷ are joined to form a 5- to 7-membered ring, —NR⁶R⁷ may represent pyrrolidin-1-yl-, 2-OH-pyrrolidin-1-yl-, 2-COOH-pyrrolidin-1-yl-, 2-CH₂OH-pyrrolidin-1-yl-, 2-CH₂OMe-pyrrolidin-1-yl-, morpholin-4-yl, 4-OH-piperidin-1-yl or 4-Me-piperazin-1-yl, especially 2-COOH-pyrrolidin-1-yl-.

When R⁶ and R⁷ are not joined, R⁶ preferably represents unsubstituted alkyl such as ethyl, propyl e.g. CHMe₂, or butyl e.g. CH₂CHMe₂, or substituted alkyl such as —CH₂COOH, —CH(ⁱPr)COOH, —C(Me)₂COOH, —C(Me)₂CH₂OH, —CH₂CH(OH)CH₂OH, —CH₂CH₂OMe, —CH₂CH₂CH₂OMe, —CH₂CH₂NMe₂, —C(Me)₂CONMe₂ or —CH(CH₂OH)COOH. Most preferably R⁶ represents —CH₂COOH, —CH(ⁱPr)COOH, —CH(^{t}Bu)COOH—, C(Me)₂COOH, C(Me)₂CONMe₂ or —CH(CH₂OH)COOH.

When R⁶ and R⁷ are not joined, R⁷ preferably represents hydrogen.

When Z represents C₁-C₆ alkoxy, examples include C₁-C₃ alkoxy e.g. —OMe, —OEt or —OCHMe₂, particularly —OEt or —OCHMe₂ e.g. —OCHMe₂.

Preferably R⁸ represents Me.

Preferably R⁹ represents Me.

Preferably R¹¹ represents Me.

Preferably R¹² represents Me.

When R¹⁰ and R¹³, together with the carbon atom to which they are attached, are joined to form a C₃-C₆ ring, examples of rings include cyclopropyl, cyclobutyl and cyclopentyl rings.

Preferably R¹⁰ represents hydrogen.

Preferably R¹³ represents hydrogen.

Preferably R¹⁴ represents hydrogen.

Preferably m represents 1 or 2, particularly 1.

Preferably a represents 1.

Preferably b represents 1.

Preferably c represents 0.

Preferably d represents 1.

Particular —YC(O)Z moieties include those present in the Examples such as —COOH, —COOⁱPr, —CH₂COOH, —CONHCMe₂COOH, —CO(pyrrolidin-1-yl), —CONHCH₂CH(OH)CH₂(OH), —CO(4-Me-piperazin-1-yl), —CO(4-OH-piperidin-1-yl), —CONHCH₂CH₂OMe, —CONHCH₂CH₂CH₂OMe, —CO(2-CH(OMe-pyrrolidin-1-yl), —NHCMe₂CH₂OH, —CO(morpholin-1-yl), —CO(2-CH₂OH-pyrrolidin-1-yl), —CONHCHMe₂, —CONHCH₂CHMe₂, —CONHEt, —CONMeⁱPr, —CONMeEt, —CONMe₂, —CONHCH(CHMeEt)COOMe, —CONH(2-OH-cyclopentyl), —CONH(4-COOH-cyclohexyl), —COOCHMe₂, —CONH^{t}Bu, —CONHCH(CH^{t}Bu)COOH, —CONHCH(CHMeEt)COOH, —CH₂CH₂COOH, —CO-(2-COOH-pyrrolidin-1-yl), —CH₂CO—(2-COOH-pyrrolidin-1-yl), —CH₂CH₂CO-(2-COOH-pyrrolidin-1-yl), —CONHCH₂COOH, —CONHCH(CHMe₂)COOH, —CONHCH(CH₂OH)COOH, —CONHCH(ⁱPr)COOH, —CONHCMe₂CONMe₂, —OCH₂COOH, —CONMeCMe₂COOH and CH₂COOEt.

When U is a bond a particular group of —YC(O)Z moieties which may be mentioned include —COOH, —CH₂COOH, —CONHCMe₂COOH, —CO(pyrrolidin-1-yl), —CONHCH₂CH(OH)CH₂(OH), —CO(4-Me-piperazin-1-yl), —CO(4-OH-piperidin-1-yl), —CONHCH₂CH₂OMe, —CONHCH₂CH₂CH₂OMe, —CO(2-CH(OMe-pyrrolidin-1-yl), —NHCMe₂CH₂OH, —CO(morpholin-1-yl), —CO(2-CH₂OH-pyrrolidin-1-yl), —CONHCHMe₂, —CONHCH₂CHMe₂, —CONHEt, —CONHCH(CHMeEt)COOMe, —CONH(2-OH-cyclopentyl), —COOCHMe₂, —CONHt-Bu, —CONHCH(CH^{t}Bu)COOH and —CONHCH(CHMeEt)COOH.

More particularly, when U is a bond, —YC(O)Z moieties include —COOH, —COOCHMe₂, —CONHCMe₂COOH, —CONHCH(CHMeEt)COOH and —CO(2-CH₂OH)-pyrrolidin-1-yl, especially —COOH, —COOCHMe₂, CONHCMe₂COOH, —CONHCH(CH^{t}Bu)COOH and —CONHCH(CHMeEt)COOH.

When U is >N-Q a particular group of —YC(O)Z moieties which may be mentioned include —COOH, —CH₂COOH, —CH₂CH₂COOH, CONHCMe₂COOH, —CO-(2-COOH-pyrrolidin-1-yl), —CH₂CO-(2-COOH-pyrrolidin-1-yl), —CH₂CH₂CO-(2-COOH-pyrrolidin-1-yl), —CONHCH₂COOH, —CONHCH(CHMe₂)COOH, —CONHCH(CH₂OH)COOH, —CONHCH(ⁱPr)COOH, CONHCH(^{t}Bu)COOH, —CONHCMe₂CONMe₂, —OCH₂COOH, —CONMeCMe₂COOH, CH₂COOEt and COOCHMe₂.

When U is a bond exemplary V moieties include a bond, CH₂, (CH₂)₂, CH(Me), CH(Me)CH₂, OCH(Me), CH₂OCH₂, NHCH₂, CH(OMe), OCH₂, 1,1- and 1,2-cyclopropyl.

Preferably Q represents hydrogen.

When Q represents hydrogen, exemplary V moieties include a bond, CH₂, (CH₂)₂ or (CH₂)₃.

Most suitably V represents a bond.

Exemplary aromatic rings that W may represent include monocylic rings such as phenyl and bicyclic rings such as naphthalene.

Exemplary heteroaromatic rings that W may represent include 5-membered monocyclic rings such as pyrrole, furan, thiophene, imidazole, pyrazole, oxazole, isoxazole, oxadiazole, thiazole and triazole; 6-membered monocyclic rings such as pyridine, pyrazine, pyridazine and pyrimidine; 9-membered bicyclic rings such as furo[3,2-c]pyridine, benzo[c]isoxazole, imidazo[1,2-a]pyridine, indole, indazole, benzothiazole, benzofuran, benzimidazole, indolizine, isoindole, benzothiophene e.g. benzo[b]thiophene; and 10-membered bicyclic rings such as quinoline, isoquinoline, quinazoline, quinoxaline, 1,8-naphthyridine, [1,6]naphthyridine, benzo[d]pyridazine and benzo[c]pyridazine

Examplary bicyclic rings which contain one unsaturated ring include indane and rings comprising an unsaturated 5- to 6-membered heteroocyclic ring containing 1 or 2 heteroatoms selected from O, N and S, for example indoline, isoindoline, chromane, isochromane, benzo[1,3]dioxane and dihydrobenzo[1,4]dioxine.

The aromatic or heteroaromatic ring of W may optionally be substituted by one or more, e.g. 1 or 2, groups as defined above. Exemplary heteroaromatic ring substituents for W include those recited above for W.

Exemplary 5- to 7-membered heterocyclic ring substituents include rings containing 1 to 3 e.g. 1 or 2 heteroatoms selected from, O, N and S such as pyrrolidine, piperidine, morpholine and piperazine as well as methyl substituted derivatives thereof such as N-methylpiperazine.

Exemplary substituted monocyclic rings that W may represent include chlorophenyl e.g. 3- or 4-chlorophenyl, dichlorophenyl e.g. 3,4-dichlorophenyl and methoxyphenyl e.g. 2-methoxyphenyl. Exemplary substituted bicyclic rings that W may represent include 6-methoxynaphthalene, 1-methoxynaphthalene, 6-fluoronaphthalene, 2,4-dimethylthiazole, 5-tert-butyl-2-methyl-2H-pyrazol-3-yl, 3-methylisoxazole, 3-methoxyisoxazole, 2-methyl-5-trifluoromethyl-2H-pyrazol-3-yl, 3-chloro-2-methoxy-pyridine, 2-hydroxypyridine, 2-methyl-2H-pyrazole-3-yl, 4-chloro-2,3-dimethyl-2H-pyrazole-3-yl, 5-ethyl-2-methyl-2H-pyrazole-3-yl, 3-isopropylisoxazole, 5-isopropyl-2-methyl-2H-pyrazole-3-yl, 5-methylisoxazole, 3-methylpyrazine, 5-methyl-2H-pyrazole-3-yl, 5-isopropyl-2H-pyrazole-3-yl, 2-methoxypyridine, 3,5-dimethoxyisoxazole, 1-methyl-4-chloro-pyrazole-5-yl, 2-methylimidazo[1,2-a]pyridine, 2-methyl-[1,6]naphthyridine, 2-methyl-2H-indazole-3-yl, 1-methyl-1H-indazole-3-yl, 3-hydroxy-quinaxoline, 2-methylquinoline, 2-methyl-1H-benzoimidazole-5-yl, 7-methoxybenzofuran, 2-methyl-1,8-naphthyridine, 4-phenylphenyl-, 3-phenylisoxazole, 5-phenylisoxazole, 5-phenyl-2H-pyrazole-3-yl, imidazol-1-yl-phenyl, 4-cyclohexylphenyl and 4-(morpholin-4-yl)phenyl.

Preferably W represents phenyl, naphthyl, benzo[c]isoxazole, pyrazole, quinoline, pyridine, isoxazole or benzofuran which rings may be optionally substituted.

When U is >N-Q a particular W groups which may be mentioned are phenyl optionally substituted by one or more groups selected from halo, hydroxy, cyano and C₁-C₆ alkyl or C₁-C₃ alkoxy either of which may be substituted by one or more fluoro atoms; and when W is optionally substituted it is preferably substituted by one or more, e.g. 1 or 2, groups selected from C₁-C₃ alkyl, trifluoromethyl, C₁-C₃ alkoxy, trifluoromethoxy, halogen and hydroxy. Specific examples of W groups include 5-chloro-2-methoxyphenyl, 5-methyl-2-methoxyphenyl, 2-methylphenyl, 2-fluorophenyl, 2-methoxyphenyl, 2-trifluoromethylphenyl and 3-methylphenyl.

When Q is joined to W such that WVNQ together forms a 5- to 7-membered nitrogen containing heterocyclic ring fused to an optionally substituted phenyl ring, exemplary heterocyclic rings include rings optionally containing an additional heteroatom selected from O, N and S. Exemplary heterocyclic rings containing no further heteroatoms include pyrrolidine, piperidine and azepine, especially pyrrolidine and piperidine. Exemplary heterocyclic rings containing further heteroatoms include piperazine and morpholine. Thus for example WVNQ may represent 1-indoline or 1-(1,2,3,4-tetrahydroquinoline).

Suitably the stereochemical orientation of the —YCOZ group to the aromatic ring across the cyclohexane ring is trans.

The molecular weight of the compounds of formula (I) is preferably less than 800, more preferably less than 600, even more preferably less than 500.

Specific compounds of the invention which may be mentioned are those included in the Examples and pharmaceutically acceptable salts and esters thereof.

As used herein, unless stated otherwise, “alkyl” as well as other groups having the prefix “alk” such as, for example, alkenyl, alkynyl, alkoxy and the like, means carbon chains which may be linear or branched or combinations thereof. Examples of alkyl groups include methyl, ethyl, propyl, isopropyl, butyl, sec- and tert-butyl, pentyl, hexyl, heptyl and the like. “Alkenyl”, “alkynyl” and other like terms include carbon chains having at least one unsaturated carbon-carbon bond.

The term “fluoroalkyl” includes alkyl groups substituted by one or more fluorine atoms, e.g. CH₂F, CHF₂ and CF₃. Fluoroalkoxy may be interpreted similarly, e.g. trifluoromethoxy.

The term “cycloalkyl” means carbocycles containing no heteroatoms, and includes monocyclic saturated carbocycles. Examples of cycloalkyl include cyclopropyl, cyclobutyl, cyclopentyl and cyclohexyl.

The term “halo” includes fluorine, chlorine, bromine and iodine atoms.

Compounds described herein may contain one or more asymmetric centers and may thus give rise to enantiomers, diastereomers and optical isomers. The present invention includes all such possible enantiomers, diastereomers as well as their racemic mixtures, their substantially pure resolved enantiomers, all possible geometric isomers, and pharmaceutically acceptable salts thereof. The above formula (I) is shown without a definitive stereochemistry at certain positions. The present invention includes all stereoisomers of formula (I) and pharmaceutically acceptable salts thereof. Further, mixtures of stereoisomers as well as isolated specific stereoisomers are also included. During the course of the synthetic procedures used to prepare such compounds, or in using racemization or epimerization procedures known to those skilled in the art, the products of such procedures can be a mixture of stereoisomers.

When a tautomer of the compound of formula (I) exists, the present invention includes any possible tautomers and pharmaceutically acceptable salts and esters thereof, and mixtures thereof, except where specifically drawn or stated otherwise.

When the compound of formula (I) and pharmaceutically acceptable salts and esters thereof exist in the form of solvates or polymorphic forms, the present invention includes any possible solvates and polymorphic forms. A type of a solvent that forms the solvate is not particularly limited so long as the solvent is pharmacologically acceptable. For example, water, ethanol, propanol, acetone or the like can be used.

The term “pharmaceutically acceptable salt” refers to salts prepared from pharmaceutically acceptable non-toxic bases or acids. When the compound of the present invention is acidic, its corresponding salt can be conveniently prepared from pharmaceutically acceptable non-toxic bases, including inorganic bases and organic bases. Salts derived from such inorganic bases include aluminum, ammonium, calcium, copper (ic and ous), ferric, ferrous, lithium, magnesium, potassium, sodium, zinc and the like salts. Particularly preferred are the ammonium, calcium, magnesium, potassium and sodium salts. Salts derived from pharmaceutically acceptable organic non-toxic bases include salts of primary, secondary, and tertiary amines, as well as cyclic amines and substituted amines such as naturally occurring and synthesized substituted amines. Other pharmaceutically acceptable organic non-toxic bases from which salts can be formed include arginine, betaine, caffeine, choline, N′,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like.

When the compound of the present invention is basic, its corresponding salt can be conveniently prepared from pharmaceutically acceptable non-toxic acids, including inorganic and organic acids. Such acids include, for example, acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, oxalic, pamoic, pantothenic, phosphoric, succinic, sulfuric, tartaric, p-toluenesulfonic acid and the like.

The term “pharmaceutically acceptable ester” refers to esters prepared from pharmaceutically acceptable non-toxic alcohols, especially labile esters that may conveniently be employed as pro-drugs. Example alcohols include, but are not limited to, methanol, ethanol, isopropanol, butanol, 2-methylpropanol, 2-methoxyethanol, 2-(dimethylamino)ethanol, 2-(diethylamino)ethanol, 2-(1-piperidinyl)ethanol, 2-(1-morpholinyl)ethanol, hydroxyacetone and the like.

It will be apparent to those skilled in the art that compounds of formula (I) where R³ is OH and Z is OH are hydroxy acids and therefore may exist as cyclic esters (lactones). These lactones, including those represented by formula (II) are encompassed within the scope of this patent.

Since the compounds of formula (I) are intended for pharmaceutical use they are preferably provided in substantially pure form, for example at least 60% pure, more suitably at least 75% pure, especially at least 98% pure (% are on a weight for weight basis).

In accordance with this invention, the compounds of formula (I) can be prepared as outlined below wherein X¹, X², R¹, R², R³, Q, V, W, Y and Z are as defined above for formula (I).

Compounds of formula (I) in which Z represents —NR⁶R⁷ may be prepared from corresponding compounds of formula (I) in which Z represents hydroxy by a process involving a conventional step of carboxylic acid to amide conversion. For example the compound of formula (I) in which Z represents hydroxy, or a protected derivative thereof, or an activated derivative thereof, such as an acid halide or anhydride, may be reacted with a compound of formula HNR⁶R⁷.

Compounds of formula (I) in which Z represents hydroxy may be prepared from corresponding compounds of formula (I) in which Z represents C₁-C₆ alkoxy or another ester forming moiety by a conventional process of ester hydrolysis under conditions of acid or base catalysis.

Compounds of formula (I), or a protected derivative thereof, may be prepared by a process comprising reaction of a compound of formula (II):

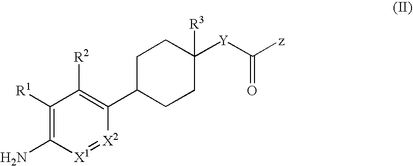

or a protected derivative thereof; with, when U is a bond, a compound of formula (III):

W—V—COOH (III)

or a protected derivative thereof or an activated derivative thereof;

or when U is >N-Q, a compound of formula (IIIA) or (IIIB)

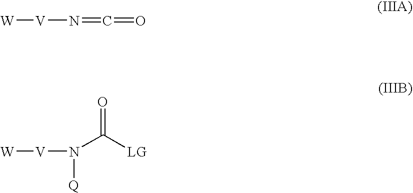

wherein LG is a leaving group, or a protected derivative of either thereof.

Examples of leaving groups include halogen e.g. Cl. Further leaving groups include carbamate leaving groups i.e. where LG=OR^{c} wherein R^{c} is typically an aromatic moiety such as substituted phenyl especially 4-nitrophenyl.

Formation of the amide by reaction of compounds of formula (II) and (III) or protected derivatives thereof may be facilitated by use of a coupling reagent such as WSC or HATU. Alternatively compounds of formula (III) may be employed in the form of an activated derivative thereof such as an acid halide or acid anhydride. Such activated derivatives may be obtained from the corresponding acid by conventional means. When a compound of formula (III) is employed as an acid halide, it may suitably be reacted with a compound of formula (II) in an inert aprotic solvent such as THF in the presence of a base such as TEA.

Compounds of formula (II) and (IIIA) or (IIIB) may suitably be combined in an inert solvent such as THF at room or elevated temperature.

Compounds of formula (IIIA) may be prepared by reacting a compound of formula (IIIC):

W—V—NH₂ (IIIC)

or a protected derivative thereof; with an isocyanate forming agent such as triphosgene.

Suitably when Z represents hydroxy the compound of formula (I) is protected as the corresponding ester i.e. a corresponding compound in which Z represents OR wherein R represents an organic moiety such as alkyl, e.g. C₁-C₆ alkyl, such as methyl or ethyl, or -alkylaryl e.g. benzyl. Thus compounds of formula (I) in which Z represents OH or else the Z group comprises a COOH moiety may be prepared by deprotection, e.g. hydrolysis, of a corresponding compound in which said Z group is protected, for example as an ester such as an alkyl, e.g. methyl or ethyl, ester.

Compounds of formula (II) or a protected derivative thereof in which Y represents a bond and R³ represents hydrogen may be prepared by the route shown in Scheme 1 below:

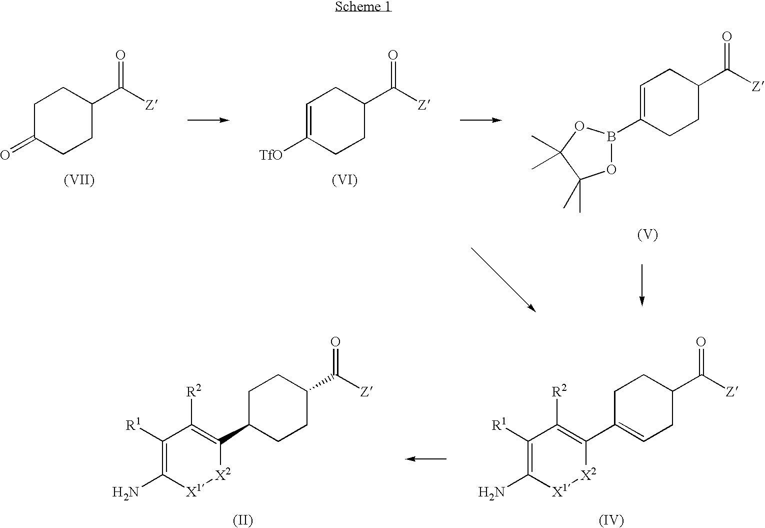

In Scheme 1, Z′ represents —NR⁶R⁷ or —OR wherein R is an ester forming group as described above.

Thus, an appropriately substituted cyclohexanone such as (VII) can be converted to the corresponding triflate (VI) via quenching of the intermediate enolate with a triflating agent such as N-phenyl triflamide. The enolate could be generated by addition of a base such as lithium diisopropylamide, typically at low temperature. Triflate (VI) can be reacted with an appropriately substituted aryl or heteroaryl boronic acid under Suzuki coupling conditions to afford cyclohexene (IV). A Suzuki coupling typically utilises a palladium catalyst in the presence of a base e.g. potassium carbonate in a solvent such as ethanol or dioxane, or in a mixture of solvents such as ethanol/water. If desired, the triflate (VI) can be converted to boronic ester (V) by reaction with bis-pinacolato-diborane in the presence of a base and palladium catalyst. Boronic ester (V) can then be reacted with an appropriately substituted aryl or heteroaryl bromide or iodide, again under Suzuki coupling conditions, to afford cyclohexene (IV). Cyclohexene (IV) can be reduced to an aniline of type (II) by, for example, reaction with hydrogen gas under palladium catalysis. This reaction could afford a mixture of stereoisomers which may be separated, if desired, as outlined previously.

Compounds of formula (II) or a protected derivative thereof in which Y represents a bond and R³ represents hydrogen may alternatively be prepared by reduction of a corresponding nitro compounds as outlined in Scheme 2 below wherein Z′ is as described in Scheme 1:

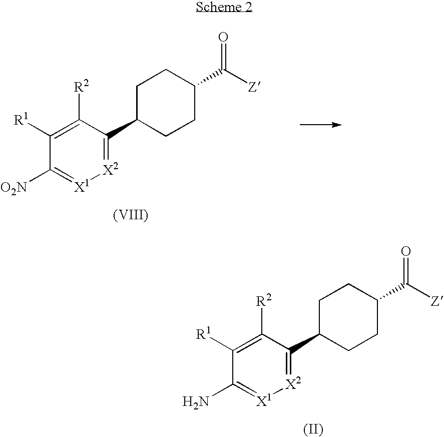

Nitrobenzene (VIII) can be reduced to aniline (II) by a variety of techniques such as hydrogenation in the presence of a palladium catalyst. If R¹ and/or R² are a halogen such as chlorine, a platinum catalyst may be employed to mitigate dehalogenation.

The corresponding nitro compounds of formula (VIII) may be prepared following Scheme 3 outlined below:

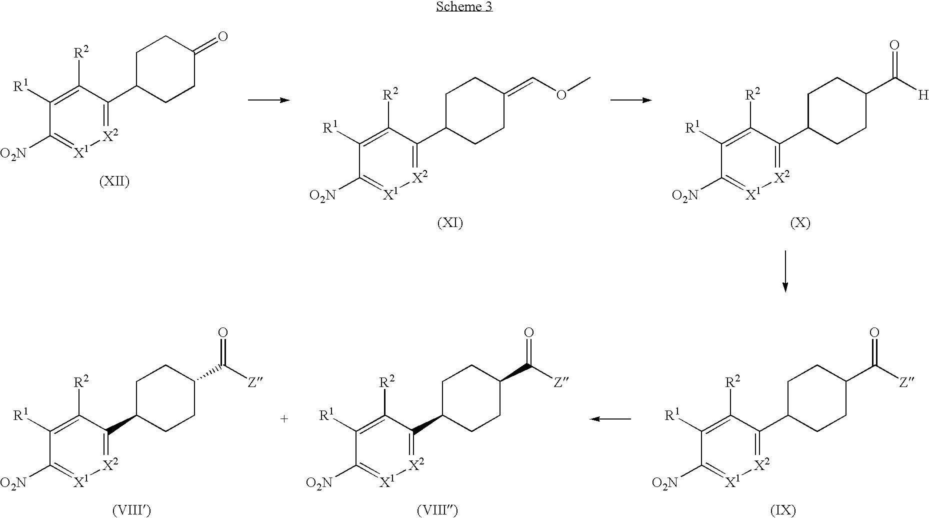

In Scheme 3, Z″ represents OR wherein R is an ester forming group as described above.

Homologation of cyclohexanone (XII) under Wittig or Horner-Wadsworth-Emmons conditions could afford enol ethers of type (XI). Subsequent hydrolysis under acidic conditions affords aldehydes of type (X). Conversion to ester (IX) can be carried out under conventional conditions. For examples esters may be prepared in one pot using Oxone™ in a suitable alcoholic solvent. Stereoisomers (VIII′) and (VIII″) can be separated using standard laboratory techniques such as column chromatography or recrystallisation. Stereochemistry can be ascertained by appropriate NMR techniques.

Compounds of formula (XII) may be prepared following Scheme 4 below:

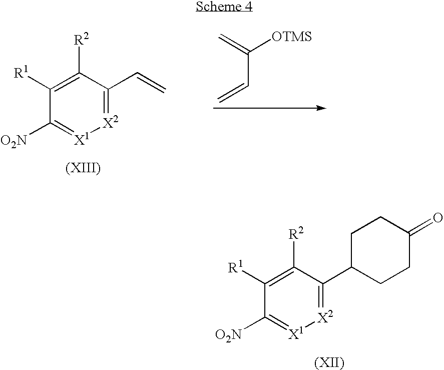

Reaction of an appropriate nitrostyrene (XIII) with 2-trimethylsilyloxybutadiene affords the crude silyl enol ether intermediate, that can be treated with dilute acid to yield the cyclohexanone (XII).

Compounds of formula (II) in which Y represents CH₂ and R³ represents hydrogen may be prepared following Scheme 5:

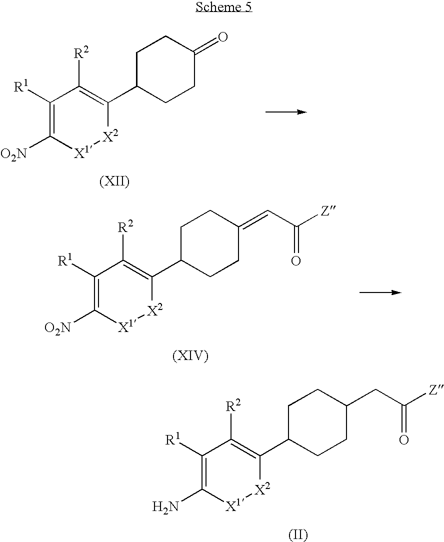

In Scheme 5, Z″ represents OR wherein R is an ester forming group as described above.

Homologation of cyclohexanone (XIV) under appropriate Wittig or Horner-Wadsworth-Emmons conditions could afford α,β-unsaturated ester (XVI). Reduction of the nitro group and double bond to yield aniline (II) could be carried out in one step, if desired, under catalytic hydrogenation conditions in the presence of a palladium catalyst. Aniline (II) may be obtained as a mixture of stereoisomers, which could be separated as outlined above.

Compounds of formula (II) in which Y represents (CH₂)₂ and (CH₂)₃ and R³ represents hydrogen may be prepared following Scheme 6 below:

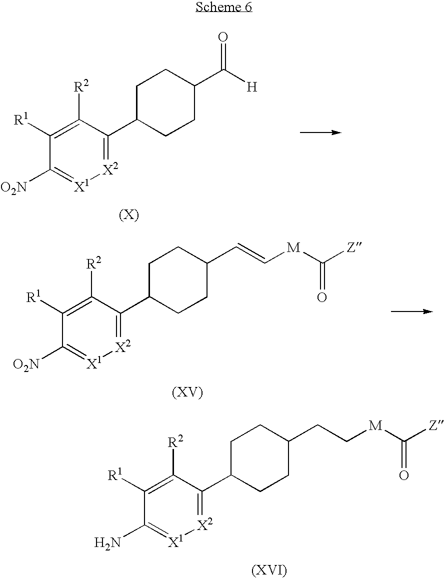

In Scheme 6, Z″ represents OR wherein R is an ester forming group as described above. Anilines of type (XVI) where M is a bond or methylene linker can be prepared from aldehyde (X) as outlined in Scheme 6. Homologation of aldehyde (X) under appropriate Wittig or Horner-Wadsworth-Emmons conditions could afford unsaturated ester (XV). Conversion to (XVI) can be carried out under catalytic hydrogenation conditions in the presence of, for example, a palladium catalyst.

Further nitro intermediates which may be used in the preparation of compounds of formula (II), e.g. intermediates of type (XVII) and (XVIII) where R³ is hydroxy or C₁-C₃ alkoxy, can be prepared as outlined in Scheme 7 below, where R^{t} represents C₁-C₃ alkyl and Z″ is as described above:

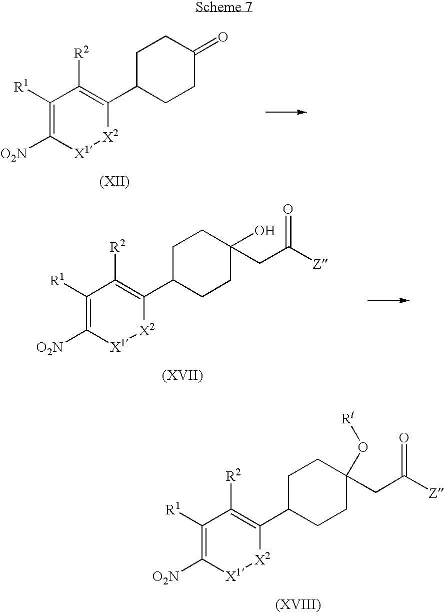

Addition of the lithium salt of methyl acetate to cyclohexanone (XII) would afford β-hydroxyester of type (XVII) as a mixture of stereoisomers. Alkylation of (XVII) with alkyl iodide (R^{t}—I) in the presence of a base could afford β-alkoxyesters of type (XVIII). R^{t} is, for example, methyl. If desired, stereoisomers could be separated by standard laboratory techniques. Compounds of type (XVIII) could be converted to the corresponding anilines by reduction of the nitro group employing, for example, hydrogenation in the presence of a palladium catalyst.

Further nitro intermediates of type (XX) and (XXI) where R^{t} represents C₁-C₃ alkyl can be prepared as outlined in Scheme 8 below:

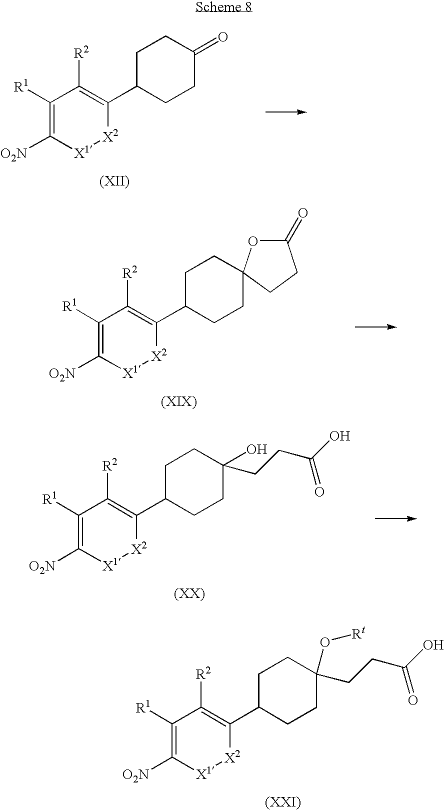

Reaction of cyclohexanone (XII) with methyl acrylate in the presence of samarium (II) iodide could afford lactone (XIX). Hydrolysis of this lactone with, for example, sodium hydroxide would afford hydroxy acid (XX) which could be alkylated with an alkyl iodide (R^{t}I) in the presence of base, to yield alkoxy acids of type (XXI).

Nitro intermediates of formula (XXIII) or a protected derivative thereof in which Y represents —O(CH₂)ₙ(CR¹⁰R¹³)ₒ(CH₂)ₚ— may be prepared as shown in Scheme 9 below:

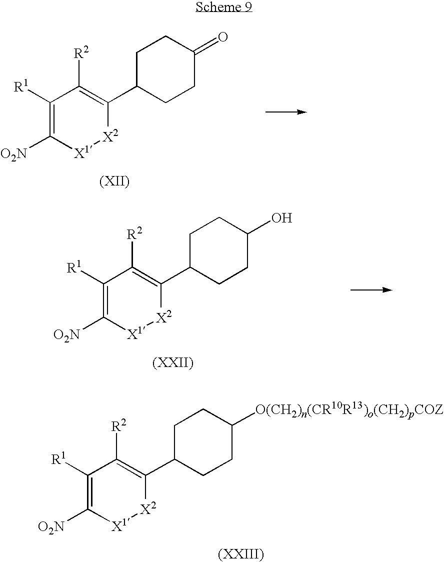

Reduction of cyclohexanone (XII) with a reducing agent such as sodium borohydride would afford a cyclohexanol of type (XXII) as a mixture of stereoisomers. Alkylation of the cyclohexanol with a compound of formula Br(CH₂)ₙ(CR¹⁰R¹³)ₒ(CH₂)ₚCOZ, or a protected derivative thereof, would afford compounds of type (XXIII). α-, β- and γ-Bromo esters are commercially available, or can be readily prepared by conventional means. Reduction of compounds of formula (XXIII) to the corresponding anilines may be carried out by employing a technique such as catalytic hydrogenation in the presence of a palladium catalyst.

Nitro intermediates of formula (XXIV) or a protected derivative thereof, in which D represents a bond or a one or two carbon linker and Z″ is as described above, may be prepared as shown in Scheme 10 below:

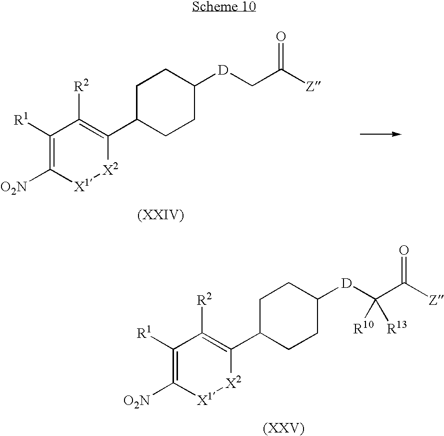

Treatment of ester (XXIV) with a strong base such as lithium diisopropylamide, typically at low temperature, affords the corresponding intermediate anion. This anion can be quenched with an alkyl halide such as methyl iodide to afford the α-alkyl ester. This protocol can be repeated to install a second alkyl group. Alternatively, if the alkyl halide contains two suitably placed halides, such as 1,3-dibromopropane, then a carbocylic ring can be installed adjacent to the ester group by this procedure.

Compounds of formula (II) may be prepared by reduction of a corresponding compound of formula (XXVI) or a protected derivative thereof:

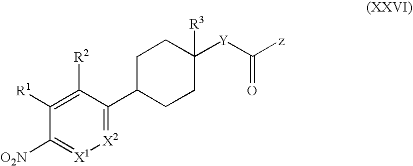

Reduction of compounds of formula (XXVI) may be carried out by employing a technique such as catalytic hydrogenation in the presence of a palladium catalyst.

Intermediates of the type (II″) where R¹ is chlorine or fluorine can be prepared by halogenation of an intermediate of type (II′) as shown in Scheme 11 below:

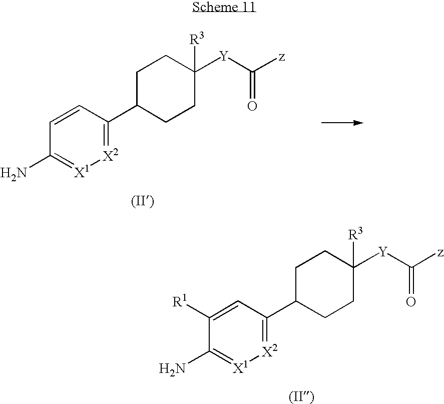

Thus reaction of (II′) with an electrophilic halogen source as N-chlorosuccinimide or SelectFluor™ generates the corresponding chloro- or fluoroanilines respectively.

Alternatively, compounds of formula (I) wherein U is >N-Q, or a protected derivative thereof, may be prepared by reaction of a compound of formula (XXVII):

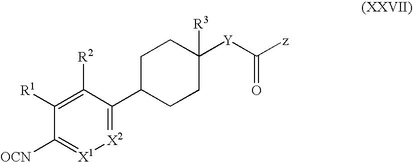

or a compound of formula (XXVIII), where LG is a leaving group as described above for compounds of formula (IIIB):

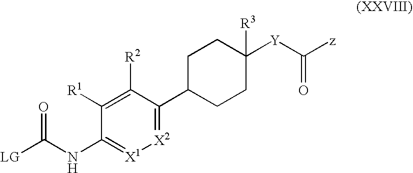

or a protected derivative of either thereof; with a compound of formula (XXIX):

W—V—NQH (XXIX)

or a protected derivative thereof, under similar conditions to those described above for the reaction of compounds of formula (II) with compounds of formula (IIIA) or (IIIB). This process is also applicable for those compounds where Q is joined to W such that WVNQ together forms a 5- to 7-membered nitrogen containing heterocyclic ring fused to phenyl which may optionally be substituted.

Compounds of formula (XXVII) and (XXVIII) may be prepared from compounds of formula (II) by an analogous process to that described above for the preparation of compounds of formula (IIIA) and (IIIB) from compounds of formula (IIIC). Thus compounds of formula (XXVII) may be prepared from compounds of formula (II) by known methods, for example carbamoyl chlorides (compounds of type (XXVIII) where LG is chlorine) may be prepared by reacting compounds of formula (II) with phosgene. Carbamoyl chlorides can lose hydrogen chloride to form isocyantes. Compounds of formula (XXVIII) may be prepared from compounds of formula (II) by known methods, for example when LG is a substituted phenol, carbamates can be prepared by reacting compounds of formula (II) with substituted phenyl chloroformates.

The skilled person will we aware that the intermediate ester compounds (Z′ or Z″═OR) in Schemes 1, 2, 3, 5, 6, 7, 9 and 10 may if desired be converted into amide analogues (Z═NR⁶R⁷) by conventional means.

Compounds of formula (III), (IIIA), (IIIB), (IIIC), (VII) and HNR⁶R⁷ are either known compounds or may be prepared by conventional means known per se or may be prepared by methods described elsewhere herein.

It will be understood that it if necessary may be desirable to employ protecting groups in organic synthesis. Examples of protecting groups and means for their removal may be obtained by reference to: “Protective Groups in Organic Synthesis”, T. W. Greene & P. G. M. Wuts, John Wiley & Sons.

Further details for the preparation of the compounds of formula (I) are found in the examples.

The compounds of formula (I) may be prepared singly or as compound libraries comprising at least 2, for example 5 to 1,000, compounds and more preferably 10 to 100 compounds of formula (I). Compound libraries may be prepared by a combinatorial “split and mix” approach or by multiple parallel synthesis using either solution or solid phase chemistry, using procedures known to those skilled in the art.

During the synthesis of the compounds of formula (I), labile functional groups in the intermediate compounds, e.g. hydroxy, carboxy and amino groups, may be protected. The protecting groups may be removed at any stage in the synthesis of the compounds of formula (I) or may be present on the final compound of formula (I). A comprehensive discussion of the ways in which various labile functional groups may be protected and methods for cleaving the resulting protected derivatives is given in, for example, Protective Groups in Organic Chemistry, T. W. Greene and P. G. M. Wuts, (1991) Wiley-Interscience, New York, 2^{nd} edition.

Any novel intermediates as defined above such as (II), (IV), (VIII) (VIII′), (IX), (X), (XI), (XII), (XIII), (XIV), (XV), (XVI), (XVII), (XVIII), (XIX), (XX), (XXI), (XXII), (XXIII), (XXIV), (XXV), (XXVI), (XXVII) and (XXVIII) are also included within the scope of the invention.

The preferences recited above for the compounds of formulae (I) also apply to any intermediate compounds.

The invention also encompasses a pharmaceutical composition comprising a compound of formula (I), or a pharmaceutically acceptable salt thereof, in combination with a pharmaceutically acceptable carrier.

Preferably the composition is comprised of a pharmaceutically acceptable carrier and a non-toxic therapeutically effective amount of a compound of formula (I), or a pharmaceutically acceptable salt thereof.

Moreover, the invention also provides a pharmaceutical composition for the treatment of disease by inhibiting DGAT1, e.g. resulting in the treatment of obesity, comprising a pharmaceutically acceptable carrier and a non-toxic therapeutically effective amount of compound of formula (I), or a pharmaceutically acceptable salt thereof.

The pharmaceutical compositions may optionally comprise other therapeutic ingredients or adjuvants. The compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions may be conveniently presented in unit dosage form and prepared by any of the methods well known in the art of pharmacy.

In practice, the compounds of formula (I), or pharmaceutically acceptable salts thereof, can be combined as the active ingredient in intimate admixture with a pharmaceutical carrier according to conventional pharmaceutical compounding techniques. The carrier may take a wide variety of forms depending on the form of preparation desired for administration, e.g. oral or parenteral (including intravenous).

Thus, the pharmaceutical compositions can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion, or as a water-in-oil liquid emulsion. In addition to the common dosage forms set out above, the compound of formula (I), or a pharmaceutically acceptable salt thereof, may also be administered by controlled release means and/or delivery devices. The compositions may be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

The compounds of formula (I), or pharmaceutically acceptable salts thereof, can also be included in pharmaceutical compositions in combination with one or more other therapeutically active compounds.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In preparing the compositions for oral dosage form, any convenient pharmaceutical media may be employed. For example, water, glycols, oils, alcohols, flavoring agents, preservatives, coloring agents, and the like may be used to form oral liquid preparations such as suspensions, elixirs and solutions; while carriers such as starches, sugars, microcrystalline cellulose, diluents, granulating agents, lubricants, binders, disintegrating agents, and the like may be used to form oral solid preparations such as powders, capsules and tablets. Because of their ease of administration, tablets and capsules are the preferred oral dosage units whereby solid pharmaceutical carriers are employed. Optionally, tablets may be coated by standard aqueous or nonaqueous techniques.

A tablet containing the composition of this invention may be prepared by compression or molding, optionally with one or more accessory ingredients or adjuvants. Compressed tablets may be prepared by compressing, in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent. Each tablet preferably contains from about 0.05 mg to about 5 g of the active ingredient and each cachet or capsule preferably containing from about 0.05 mg to about 5 g of the active ingredient.

For example, a formulation intended for the oral administration to humans may contain from about 0.5 mg to about 5 g of active agent, compounded with an appropriate and convenient amount of carrier material which may vary from about 5 to about 95 percent of the total composition. Unit dosage forms will generally contain between from about 1 mg to about 2 g of the active ingredient, typically 25 mg, 50 mg, 100 mg, 200 mg, 300 mg, 400 mg, 500 mg, 600 mg, 800 mg, or 1000 mg.

Pharmaceutical compositions of the present invention suitable for parenteral administration may be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g. glycerol, propylene glycol and liquid polyethylene glycol), vegetable oils, and suitable mixtures thereof.

Pharmaceutical compositions of the present invention can be in a form suitable for topical use such as, for example, an aerosol, cream, ointment, lotion, dusting powder, or the like. Further, the compositions can be in a form suitable for use in transdermal devices. These formulations may be prepared, using a compound of formula (I), or a pharmaceutically acceptable salt thereof, via conventional processing methods. As an example, a cream or ointment is prepared by admixing hydrophilic material and water, together with about 5 wt % to about 10 wt % of the compound, to produce a cream or ointment having a desired consistency.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories may be conveniently formed by first admixing the composition with the softened or melted carrier(s) followed by chilling and shaping in molds.

In addition to the aforementioned carrier ingredients, the pharmaceutical formulations described above may include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient.

Compositions containing a compound of formula (I), or pharmaceutically acceptable salts thereof, may also be prepared in powder or liquid concentrate form.

Generally, dosage levels on the order of 0.01 mg/kg to about 150 mg/kg of body weight per day are useful in the treatment of the above-indicated conditions, or alternatively about 0.5 mg to about 7 g per patient per day. For example, obesity may be effectively treated by the administration of from about 0.01 to 50 mg of the compound per kilogram of body weight per day, or alternatively about 0.5 mg to about 3.5 g per patient per day.

It is understood, however, that the specific dose level for any particular patient will depend upon a variety of factors including the age, body weight, general health, sex, diet, time of administration, route of administration, rate of excretion, drug combination and the severity of the particular disease undergoing therapy.

The compounds of the invention may be used for treating diseases in which inhibition of DGAT1 is desirable, such as obesity.

The compounds of the invention may also be used for treating of other diseases in which obesity is a factor including metabolic diseases such as Type II diabetes, metabolic syndrome (syndrome X), impaired glucose tolerance, dyslipidemia, hyperlipidemia, hypertriglyceridemia, hypercholesterolemia, low HDL levels and hypertension. The compounds may also be used for treating other disorders associated with DGAT1, such as acne.

The invention also provides a method for the treatment of obesity comprising a step of administering to a subject in need thereof an effective amount of a compound of formula (I), or a pharmaceutically acceptable salt thereof.

The invention also provides a method for the treatment of a metabolic disease selected from Type II diabetes, metabolic syndrome (syndrome X), impaired glucose tolerance, dyslipidemia, hyperlipidemia, hypertriglyceridemia, hypercholesterolemia, low HDL levels and hypertension, comprising a step of administering to a subject in need thereof an effective amount of a compound of formula (I), or a pharmaceutically acceptable salt thereof.

The invention also provides the use of a compound of formula (I), or a pharmaceutically acceptable salt thereof, in the treatment of a condition as defined above.

The invention also provides the use of a compound of formula (I), or a pharmaceutically acceptable salt thereof, in the manufacture of a medicament for the treatment of a condition as defined above.

In the methods of the invention the term “treatment” includes both therapeutic and prophylactic treatment.

The compounds of formula (I), or pharmaceutically acceptable salts thereof, may be administered alone or in combination with one or more other therapeutically active compounds. The other therapeutically active compounds may be for the treatment of the same disease or condition as the compounds of formula (I), or a different disease or condition. The therapeutically active compounds may be administered simultaneously, sequentially or separately.

The compounds of formula (I), may be administered with other active compounds for the treatment of obesity and/or diabetes, for example insulin and insulin analogs, gastric lipase inhibitors, pancreatic lipase inhibitors, sulfonyl ureas and analogs, biguanides, α2 agonists, glitazones, PPAR-γ agonists, RXR agonists, fatty acid oxidation inhibitors, α-glucosidase inhibitors, β-agonists, phosphodiesterase inhibitors, lipid lowering agents, glycogen phosphorylase inhibitors, MCH-1 antagonists and CB-1 antagonists, amylin antagonists, lipoxygenase inhibitors, somostatin analogs, glucokinase activators, glucagon antagonists, insulin signalling agonists, PTP1B inhibitors, gluconeogenesis inhibitors, antilypolitic agents, GSK inhibitors, galanin receptor agonists, anorectic agents, CCK receptor agonists, leptin, serotonergic/dopaminergic antiobesity drugs, CRF antagonists, CRF binding proteins, thyromimetic compounds, aldose reductase inhibitors, glucocorticoid receptor antagonists, NHE-1 inhibitors or sorbitol dehydrogenase inhibitors.

When used in combination therapy, the compounds of formula (I) are preferably administered in combination with other non-central approaches to obesity e.g. with orlistat (Xenical®) or a with an agonist of GPR119 (GPR119 is identified as SNORF25 in WO00/50562 which discloses both the human and rat receptors and in U.S. Pat. No. 6,468,756 which also discloses the mouse receptor) if peripherally acting.

All publications, including, but not limited to, patents and patent application cited in this specification, are herein incorporated by reference as if each individual publication were specifically and individually indicated to be incorporated by reference herein as fully set forth.

The invention will now be described by reference to the following examples which are for illustrative purposes and are not to be construed as a limitation of the scope of the present invention.

### EXAMPLES

### Abbreviations

DBU: 1,8-diazobicyclo[5,4,0]undec-7-ene; DCM: dichloromethane; DMF: dimethylformamide; h: hour; HATU: O-(7-Azabenzotriazol-1-yl)-N,N,N′,N′-tetramethyluronium hexafluorophosphate; HOBt: 1-hydroxybenzotriazole; min: minute; MP: macroporous; RT: retention time; TEA: triethylamine; TFA: trifluoroacetic acid; THF: tetrahydrofuran; WSC: 1-ethyl-3-(3-dimethylaminopropyl)carbodiimide hydrochloride.

### Materials and Methods

### LCMS Method 1

LCMS data were obtained as follows: Waters Atlantis C18, 3μ (3.0×20 mm, flow rate 0.85 mL/min) eluting with a H₂O-MeCN gradient containing 0.1% v/v HCO₂H over 6.5 min with UV detection at 220 nm Gradient information: 0.0-0.3 min 100% H₂O; 0.3-4.25 min: Ramp to 10% H₂O-90% CH₃CN; 4.25 min-4.4 min: Ramp to 100% CH₃CN; 4.4-4.9 min: Hold at 100% MeCN; 4.9-5.0 min: Return to 100% H₂O; 5.00-6.50 min: Hold at 100% H₂O. The mass spectra were obtained using an electrospray ionisation source in either the positive (ESI⁺) ion or negative ion (ESI⁻) mode.

### LCMS Method 2

Waters Xterra MS C18, 5 μm (4.6×50 mm, flow rate 1.5 mL/min) eluting with a H₂O-MeCN gradient containing 0.1% v/v ammonia over 12 minutes with UV detection at 215 and 254 nm Gradient information: 0.0-8.0 min: Ramp from 95% H₂O-5% MeCN to 5% H₂O-95% MeCN; 8.0-9.9 min: Hold at 5% H₂O-95% MeCN; 9.9-10.0 min: Return to 95% H₂O-5% MeCN; 10.0-12.0 min: Hold at 95% H₂O-5% MeCN. Mass spectra were obtained using an electrospray ionization source in either the positive (ESL⁺) or negative (ESI⁻) mode.

Unless otherwise stated, LCMS method 1 was employed.

### Synthesis of Intermediates

### Intermediate 1: [4-(4-Nitrophenyl)cyclohexylidene]acetic acid ethyl ester

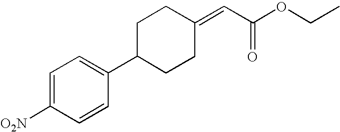

To a suspension of triethylphosphonoacetate (6.7 g, 30 mmol) in DMF (20 mL) cooled in an ice/water bath was added sodium hydride (1.18 g as a 60% suspension in oil) portion-wise. After 30 min 4-(4-nitrophenyl)cyclohexanone (5.98 g) was added, and stirring continued for a further 45 min. The reaction mixture was poured into 0.5M hydrochloric acid solution and extracted with ethyl acetate. The organic layer was separated and washed with water, sodium hydrogen carbonate solution and brine. The solution was then dried (MgSO₄) and concentrated. The resulting residue was washed twice with petroleum ether to afford the title compound. δ_{H} 400 MHz (CDCl₃) 1.32 (3H, t), 1.78 (2H, m), 2.15 (2H, m), 2.23 (2H, m), 2.97 (1H, m), 4.06 (1H, m), 4.20 (2H, q), 5.77 (1H, s), 7.40 (2H, d) and 8.21 (2H, d).

### Intermediate 2: trans-[4-(4-Aminophenyl)cyclohexyl]acetic acid ethyl ester

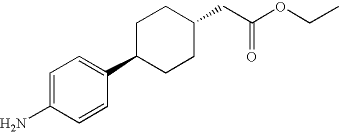

A mixture of [4-(4-nitrophenyl)cyclohexylidene]acetic acid ethyl ester and palladium on carbon (50 mg, 10%) was stirred rapidly under hydrogen atmosphere overnight. The mixture was filtered through celite and concentrated. Trituration with toluene/petroleum ether afforded the title compound. δ_{H} 400 MHz (CDCl₃) 1.18 (2H, m), 1.30 (3H, t), 1.49 (2H, m), 1.75 (4H, m), 2.25 (2H, d), 2.40 (1H, m), 3.58 (1H, s), 4.19 (2H, q), 6.68 (2H, d) and 7.03 (2H, d).

### Intermediate 3: 4-(4-Nitrophenyl)cyclohexanecarbaldehyde

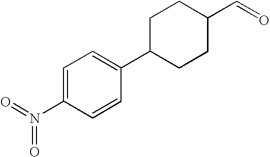

To a suspension of (methoxymethyl)triphenylphosphonium chloride (12.5 g) in THF (40 mL) was added potassium t-butoxide (2.05 g). After stirring for 90 min, 4-(4-nitrophenyl)cyclohexanone (2 g) was added the mixture heated under reflux for 17 h. The mixture was cooled, poured into saturated aqueous sodium hydrogen carbonate solution and extracted with twice with diethyl ether. The combined extracts were washed with water, brine, dried and concentrated. The resulting residue was triturated with diethyl ether and filtered. The filtrate was concentrated, dissolved in a mixture of acetic acid/water (4:1) and heated to 70° C. for 210 min. The reaction mixture was cooled, concentrated and purified via flash chromatography to afford the title compound as a 1:1.7 mixture of isomers. δ_{H} 400 MHz (CDCl₃) 1.42-1.63 (4H, m), 1.73-1.90 (2H, m), 2.09 (1H, m), 2.21 (1H, m), 2.32-2.45 (1H, m), 2.59-2.73 (1H, m), 7.34 (0.74H, d), 7.40 (1.26H, d), 8.18 (2H, m), 9.73 (0.63H, s) and 9.82 (0.37H, s).

### Intermediate 4: (E)-3-[4-(4-Nitrophenyl)cyclohexyl]acrylic acid methyl ester and 3-[4-(4-nitrophenyl)cyclohexylidene]propionic acid methyl ester

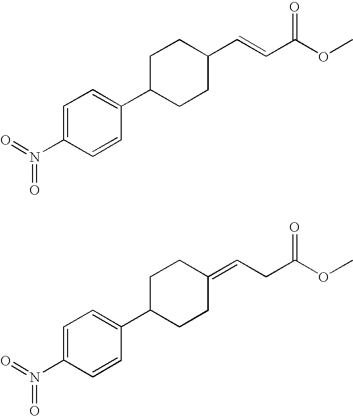

To a solution of 4-(4-nitrophenyl)cyclohexanecarbaldehyde (1.3 g) in toluene (50 mL) was added DBU (1 mL) and the mixture heated at 50° C. for 5 h. (Carboxymethoxy-methylene)triphenylphosphorane (2.79 g) was then added portion-wise and the reaction mixture heated to 100° C. for 17 h. The mixture was cooled, diluted with ethyl acetate and washed sequentially with aqueous potassium hydrogen sulphate solution (5%), saturated aqueous sodium hydrogen carbonate solution, brine, dried (MgSO₄) and concentrated. Flash chromatography afforded the title compounds as a mixture. MH⁺290.05, RT 5.37 min.

### Intermediate 5: 3-[4-(4-Aminophenyl)cyclohexyl]propionic acid methyl ester

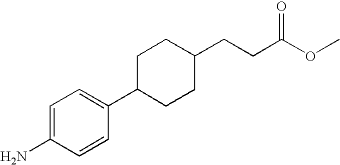

A suspension of a mixture of (E)-3-[4-(4-nitrophenyl)cyclohexyl]acrylic acid methyl ester and 3-[4-(4-nitrophenyl)cyclohexylidene]propionic acid methyl (1.41 g) and palladium on carbon (10%, 0.14 g) in ethanol (30 mL) was stirred rapidly under hydrogen atmosphere for 17 h. The mixture was filtered through celite and concentrated to afford the title compound. MH⁺262.08, RT 3.62 min.

### Intermediate 6: trans and cis-4-(4-Nitrophenyl)cyclohexanecarboxylic acid isopropyl ester

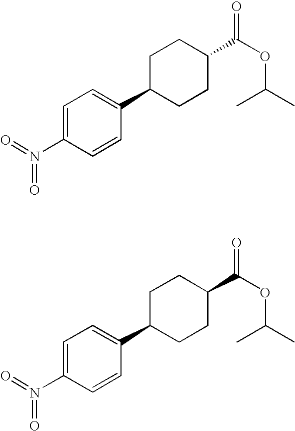

To a solution of 4-(4-nitrophenyl)cyclohexanecarbaldehyde (10 g) in isopropanol (430 mL) was added Oxone™ (26.35 g). After stirring for 17 h, the mixture was poured into 1M hydrochloric acid and extracted twice with DCM. The combined organic extracts were washed with brine, dried (MgSO₄) and concentrated. The residue was purified by flash chromatography (eluent hexane, 4% ethyl acetate) to afford cis-4-(4-nitrophenyl)cyclohexanecarboxylic acid isopropyl ester. δ_{H} 400 MHz (CDCl₃) 1.30 (6H, d) 1.64-1.86 (6H, m), 2.30 (2H, m), 2.65-2.76 (2H, m), 5.12 (1H, septet), 7.38 (2H, d) and 7.18 (2H, d).

Further elution with the same solvent afford trans-4-(4-nitrophenyl)cyclohexane-carboxylic acid isopropyl ester. δ_{H} 400 MHz (CDCl₃) 1.28 (6H, d), 1.47-1.70 (4H, m), 2.03 (2H, m), 2.17 (2H, m), 2.36 (1H, m), 2.68 (1H, m), 5.06 (1H, septet), 7.39 (2H, d) and 8.19 (2H, d).

### Intermediate 7: trans-4-(4-Aminophenyl)cyclohexanecarboxylic acid isopropyl ester

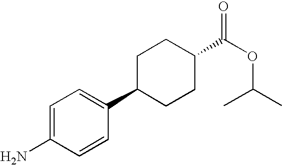

A suspension of trans-4-(4-nitrophenyl)cyclohexanecarboxylic acid isopropyl ester (5.37 g) and palladium on carbon (10%, 0.5 g) in ethanol (50 mL) was stirred rapidly under hydrogen atmosphere for 17 h. The mixture was filtered through celite and concentrated to afford the title compound. MH⁺262.09, RT 2.69 min.

### Intermediate 8: cis-4-(4-Aminophenyl)cyclohexanecarboxylic acid isopropyl ester

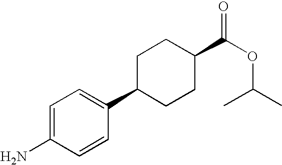

A suspension of cis-4-(4-nitrophenyl)cyclohexanecarboxylic acid isopropyl ester (0.82 g) and palladium on carbon (10%, 0.08 g) in ethanol (15 mL) was stirred rapidly under hydrogen atmosphere for 17 h. The mixture was filtered through celite and concentrated to afford the title compound. MH⁺262.07, RT 2.74 min.

### Intermediate 9: trans-4-(4-Amino-3-chlorophenyl)cyclohexanecarboxylic acid isopropyl ester

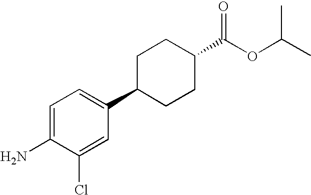

A solution of trans-4-(4-aminophenyl)cyclohexanecarboxylic acid isopropyl ester (1 g) in acetonitrile was heated to 60° C. and N-chlorosuccinimide (0.51 g) was added. After heating for 2 h the mixture was cooled, concentrated and re-dissolved in ethyl acetate. The solution was washed with 2M ammonia solution, brine, dried (MgSO₄) and concentrated. Flash chromatography afforded the title compound. MH⁺295.07, RT 3.94 min.

### Intermediate 10: trans-4-(4-Acetylaminophenyl)cyclohexanecarboxylic acid isopropyl ester

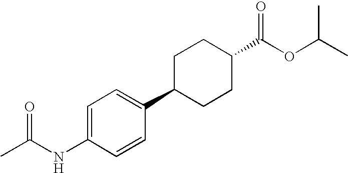

To a solution of trans-4-(4-aminophenyl)cyclohexanecarboxylic acid isopropyl ester (1 g) and triethylamine (1.6 mL) in DCM (10 mL) was added acetic anhydride (0.72 mL). After 3 h the reaction mixture was diluted with ethyl acetate and washed with water, 2M hydrochloric acid and brine. The solution was dried (MgSO₄) and concentrated to afford the title compound. MH⁺304.16, RT 3.51 min.

### Intermediate 11: trans-4-(4-Acetylamino-3-fluorophenyl)cyclohexanecarboxylic acid isopropyl ester

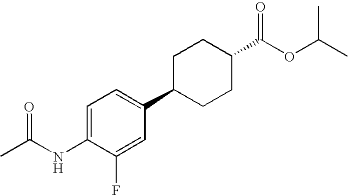

To a suspension SelectFluor™ (0.584 g) in acetonitrile was added trans-4-(4-acetylaminophenyl)cyclohexanecarboxylic acid isopropyl ester (0.5 g) and the mixture heated to reflux for 48 h. The mixture was allowed to cool to room temperature and TFA (2 mL) added. The mixture was then heated to reflux for 17 h. The reaction mixture was cooled, concentrated and the residue re-dissolved in ethyl acetate. The solution was washed with saturated sodium hydrogen carbonate solution, water and dried (MgSO₄). The mixture was then concentrated and purified by flash chromatography to obtain the title compound. MH⁺322.06, RT 3.61 min.

### Intermediate 12: trans-4-(4-Amino-3-fluorophenyl)cyclohexanecarboxylic acid isopropyl ester

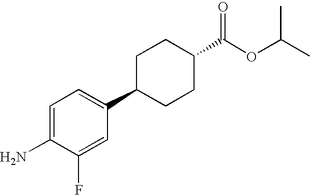

A solution of trans-4-(4-acetylamino-3-fluorophenyl)cyclohexanecarboxylic acid isopropyl ester (0.2 g) in hydrochloric acid (5 mL) and isopropanol (2 mL) was heated to 75° C. for 17 h. The mixture was cooled, concentrated, the residue re-dissolved in isopropanol (20 mL) and concentrated sulphuric acid (0.5 mL) added. The mixture was heated to reflux for 1 h, then cooled and concentrated. The resulting residue was re-dissolved in ethyl acetate, washed with saturated sodium hydrogen carbonate solution, dried (MgSO₄) and concentrated to afford the title compound. MH⁺279.06, RT 3.76 min.

### Intermediate 13: trans-2-{[4-(4-Nitrophenyl)cyclohexanecarbonyl]amino}-2-methylpropionic acid methyl ester

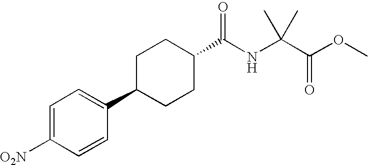

To a solution of trans-4-(4-nitrophenyl)cyclohexanecarboxylic acid (3.32 mmol) in DMF was added WSC (6.64 mmol), HOBt (6.64 mmol) and triethylamine (13.3 mmol). After stirring for 30 min, 2-amino-2-methylpropionic acid methyl ester hydrochloride (6.64 mmol) was added and stirring continued overnight. The mixture was diluted with ethyl acetate and washed with water, saturated sodium hydrogen carbonate solution and brine. The organic layer was dried (MgSO₄) and concentrated to obtain the title compound. MH⁺349.07, RT 3.44 min.

### Intermediate 14: trans-2-{[4-(4-Aminophenyl)cyclohexanecarbonyl]amino}-2-methylpropionic acid methyl ester

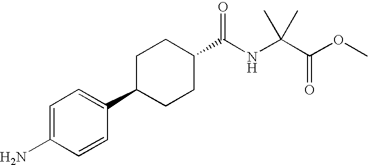

A suspension of trans-2-{[4-(4-nitrophenyl)cyclohexanecarbonyl]amino}-2-methyl-propionic acid methyl ester (0.85 g) and palladium on carbon (10%, 0.09 g) in ethanol (20 mL) was stirred rapidly under hydrogen atmosphere for 17 h. The mixture was filtered through celite and concentrated to afford the title compound. MH⁺319.07, RT 2.17 min.

### Intermediate 15: trans-4-{4-[(Benzo(c)isoxazole-3-carbonyl)amino]phenyl}cyclohexane-carboxylic acid isopropyl ester

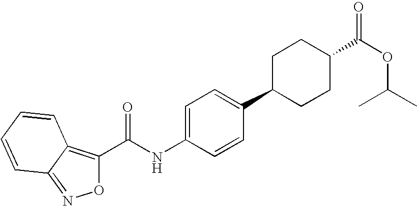

To a solution of benzo[c]isoxazole-3-carboxylic acid (4.59 mmol) in DMF was added WSC (6.38 mmol), HOBt (6.38 mmol) and triethylamine (6.38 mmol). After stirring for 30 min, trans-4-(4-aminophenyl)cyclohexanecarboxylic acid isopropyl ester (3.83 mmol) was added. After stirring for 17 h, the mixture was diluted with ethyl acetate and washed with water, saturated sodium hydrogen carbonate solution and brine. The organic layer was dried (MgSO₄) and concentrated to obtain the title compound. MH⁺407.05, RT 4.34 min.

### Intermediate 16: trans-4-{4-[(Benzo(c)isoxazole-3-carbonyl)amino]phenyl}cyclohexane-carboxylic acid

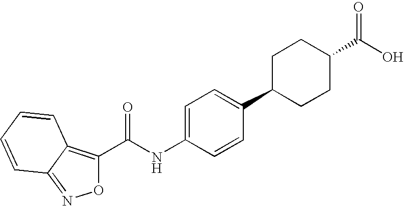

To a solution of trans-4-{4-[(benzo(c)isoxazole-3-carbonyl)amino]phenyl}-cyclohexanecarboxylic acid isopropyl ester (3.25 mmol) in THF/methanol (1:1, 20 mL) was added 1M sodium hydroxide (13 mmol). After stirring for 3 days the mixture was diluted with water and extracted three times with diethyl ether. The aqueous layer was acidified by addition of concentrated hydrochloric acid and extracted three times with ethyl acetate. The combined organic extracts were washed with brine, dried (MgSO₄) and concentrated. Flash chromatography afforded the title compound. MH⁺365.07, RT 3.54 min.

### Intermediate 17

trans-4-(4-Nitrophenyl)cyclohexanecarboxylic acid was prepared in a similar manner to Intermediate 16, RT 4.24 min.

### Intermediate 18: trans-4-[4-(3,4-Dichlorobenzoylamino)phenyl]cyclohexanecarboxylic acid isopropyl ester

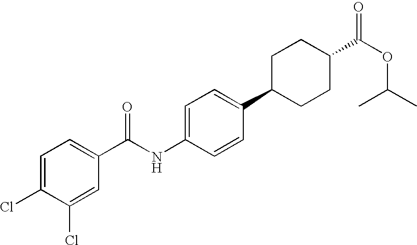

To a solution of trans-4-(4-aminophenyl)cyclohexanecarboxylic acid isopropyl ester (1.91 mmol) and triethylamine (3.83 mmol) in DCM (10 mL) was added 3,4-dichlorobenzoyl chloride (3.83 mmol). After 1 h the reaction was diluted with ethyl aceate and washed with 1M hydrochloric acid and 1M sodium hydroxide. The organic extract was dried (MgSO₄) and concentrated. Trituration with diethyl ether afforded the title compound. MH⁺433.99, RT 4.41 min.

### Intermediate 19: trans-4-[4-(3,4-Dichlorobenzoylamino)phenyl]cyclohexanecarboxylic acid

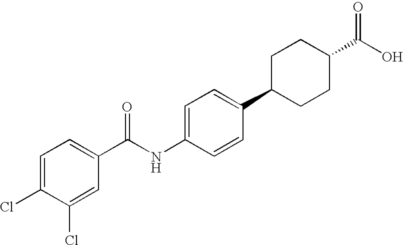

To a solution of trans-4-[4-(3,4-dichlorobenzoylamino)phenyl]cyclohexanecarboxylic acid isopropyl ester (12.4 mmol) in THF/methanol (1:1, 20 mL) was added 1M sodium hydroxide (20 mmol). After stirring for overnight the mixture was diluted with water and extracted three times with diethyl ether. The aqueous layer was acidified by addition of concentrated hydrochloric acid and extracted three times with ethyl acetate. The combined organic extracts were washed with brine, dried (MgSO₄) and concentrated to afford the title compound. MH⁺391.91, RT 3.82 min.

### Intermediate 20: 4-(4,4,5,5-Tetramethyl-[1,3,2]dioxaborolan-2-yl)cyclohex-3-ene-carboxylic acid ethyl ester

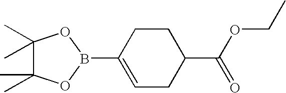

Lithium diisopropylamine (44.1 mmol) was added to a solution of 4-oxo-cyclohexanecarboxylic acid ethyl ester (29.4 mmol) in THF (100 mL) at −78° C. After 20 min, N-phenyl triflimide (44.1 mmol) in THF (50 mL) was added and stirring continued for a further 10 min before the cooling bath was removed and the mixture allowed to reach room temperature. After 2.5 h the mixture was diluted with diethyl ether and washed sequentially with 1M sodium hydroxide and 1M hydrochloric acid. The solution was dried (MgSO₄), concentrated and purified by flash chromatography. A suspension of this material, bis-dipinacolato diborane (5.96 g), potassium acetate (6.26 g), [1,1′-bis(diphenylphosphino)ferrocene]dichloropalladium(II) (0.45 g) and 1,1′-bis(diphenylphosphino)ferrocene (0.306 g) in dioxane was heated to reflux overnight. The mixture was cooled, diluted with ethyl acetate and washed with water and brine. The solution was dried (MgSO₄) concentrated and purified by chromatography to afford the title compound. MH⁺281.14, RT 3.82 min.

### Intermediate 21: trans-4-(6-Aminopyridin-3-yl)cyclohexanecarboxylic acid ethyl ester

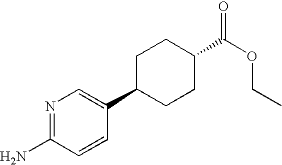

To a solution of 5-iodopyridin-2-ylamine (6.05 mmol) and 4-(4,4,5,5-tetramethyl-[1,3,2]dioxaborolan-2-yl)cyclohex-3-enecarboxylic acid ethyl ester (6.65 mmol) in anhydrous DMF (10 mL) were added tetrakis(triphenylphospine)-palladium (0) (10 mole %) and cesium carbonate (6.65 mmol). The solution was degassed under argon and heated to 100° C. for 17 h. The mixture was cooled, diluted with ethyl acetate and washed with water. A suspension of this material and palladium on carbon (10%, 0.1 g) in ethyl acetate (20 mL) was stirred rapidly under hydrogen atmosphere for 17 h. The mixture was filtered through, celite, concentrated and purified by flash chromatography. This material was dissolved in ethanol (20 mL), sodium ethoxide (1.17 g) added and the mixture heated to reflux for 5 h. The mixture was cooled, diluted with ethyl acetate and washed with 2M hydrochloric acid. The solution was dried (MgSO₄) and concentrated to obtain a solid. Trituration with DCM afforded the title compound. MH⁺249.01, RT 2.40 min.

### Intermediate 22

trans-4-(5-Aminopyridin-2-yl)cyclohexanecarboxylic acid methyl ester was prepared in an analogous manner to Intermediate 21. MH⁺248.98, RT 2.11 min.

### Intermediate 23: syn-[1-Hydroxy-4-(4-nitrophenyl)cyclohexyl]acetic acid methyl ester

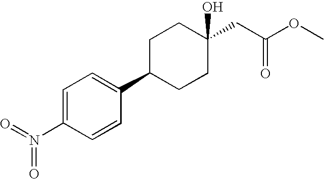

An oven-dried 3-necked flask under argon was charged with THF and lithium hexamethyldisilazane (1M, 11.4 mL) and cooled to −78° C. Methyl acetate (0.91 mL) was added and stirring continued for a further 20 min before a solution of 4-(4-nitrophenyl)cyclohexanone in THF was added drop-wise. After 90 min 1M hydrochloric acid was added, the mixture allowed to reach room temperature and extracted three times with ethyl acetate. The combined organic extracts were washed with brine, dried and concentrated. The residue was purified by flash chromatography (eluent hexane, 60% ethyl acetate) to afford syn-[1-hydroxy-4-(4-nitrophenyl)cyclohexyl]acetic acid. δ_{H} 400 MHz (CDCl₃) 1.50 (2H, m), 1.75 (2H, m), 1.92-2.18 (4H, m), 2.56 (2H, s), 2.63 (1H, m), 3.44 (1H, s), 3.78 (3H, s), 7.44 (2H, d) and 8.19 (2H, d).

### Intermediate 24: syn-[4-(4-Aminophenyl)-1-hydroxycyclohexyl]acetic acid methyl ester

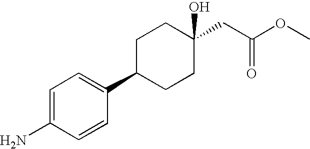

A suspension of syn-[1-hydroxy-4-(4-nitrophenyl)cyclohexyl]acetic acid methyl ester (0.195 g) and palladium on carbon (20 mg) in ethanol was stirred rapidly under hydrogen atmosphere for 17 h. The mixture was filtered through celite and concentrated to afford the title compound. MH⁺264.03, RT 1.90 min.

### Intermediate 25: syn-[1-Methoxy-4-(4-nitrophenyl)cyclohexyl]acetic acid methyl ester

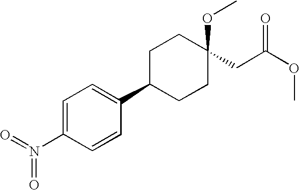

To a stirred solution of syn-[1-hydroxy-4-(4-nitrophenyl)cyclohexyl]acetic acid methyl ester (0.34 mmol) and tetrafluoroboric acid (0.34 mmol) in DCM (5 mL) at 0° C. was added trimethylsilyldiazomethane (0.61 mmol) dropwise. The reaction was neutralised by addition of triethylamine, concentrated and re-dissolved in ethyl acetate. This was washed with brine, dried (MgSO₄) and concentrated. Flash chromatography afforded the title compound. MH⁺308.05, RT 3.67 min.

### Intermediate 26: syn-[4-(4-Aminophenyl)-1-methoxycyclohexyl]acetic acid methyl ester

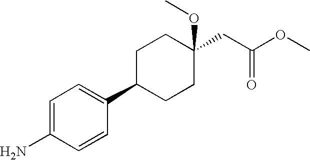

A suspension of syn-[1-hydroxy-4-(4-nitrophenyl)cyclohexyl]acetic acid methyl ester (0.195 g) and palladium on carbon (20 mg) in ethanol was stirred rapidly under hydrogen atmosphere for 3 h. The mixture was filtered through celite and concentrated and purified by flash chromatography to afford the title compound. MH⁺278.07, RT 2.27 min.

### Intermediate 27: trans-4-{4-[3-(2-Methoxy-5-methylphenyl)ureido]phenyl}cyclohexane-carboxylic acid isopropyl ester

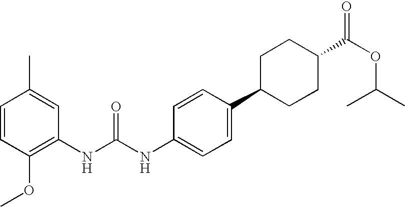

A solution of trans-4-(4-aminophenyl)cyclohexanecarboxylic acid isopropyl ester (2.3 mmol) and 2-Isocyanato-1-methyloxy-4-methylbenzene (2.5 mmol) in THF (5 mL) was stirred overnight. The mixture was diluted with DCM (5 mL) and MP-trisamine (5 mmol) added. After shaking for 3 h, the mixture was filtered and concentrated to obtain the title compound. MH⁺425.14, RT 4.26 min.

### Intermediates 28-38

The following intermediates were prepared by an analogous procedure to Intermediate 27:

trans-3-(4-{4-[3-(2-Methoxy-5-methylphenyl)ureido]phenyl}cyclohexyl)propionic acid methyl ester; MH⁺425.09, RT 4.02 min.

trans-(4-{4-[3-(5-Chloro-2-methoxyphenyl)ureido]phenyl}cyclohexyl)acetic acid ethyl ester; MH⁺445.01, RT 4.34 min.

trans-4-[4-(3-Phenylureido)phenyl]cyclohexanecarboxylic acid methyl ester; MH⁺353.06, RT 3.67 min.

trans-4-{4-[3-(2-Methoxy-5-methylphenyl)ureido]phenyl}cyclohexanecarboxylic acid methyl ester; MH⁺397.06, RT 3.89 min.

trans-3-{4-[4-(3-Phenylureido)phenyl]cyclohexyl}propionic acid methyl ester; MH⁺381.08, RT 3.97 min.

syn-(1-Hydroxy-4-{4-[3-(2-methoxy-5-methylphenyl)ureido]phenyl}cyclohexyl)acetic acid methyl ester; MH⁺427.11, RT 3.49 min.

syn-(1-Methoxy-4-{4-[3-(2-methoxy-5-methylphenyl)ureido]phenyl}cyclohexyl)acetic acid methyl ester; MH⁺441.12, RT 3.90 min.

trans-(4-{4-[3-(2-Methoxy-5-methylphenyl)ureido]phenyl}cyclohexyloxy)acetic acid methyl ester; MH⁺427.13, RT 3.84 min.

trans-2-[(4-{4-[3-(2-Methoxy-5-methylphenyl)ureido]phenyl}cyclohexanecarbonyl)-methylamino]-2-methylpropionic acid methyl ester; MH⁺496.11, RT 3.84.

trans-4-{6-[3-(2-Methoxy-5-methylphenyl)ureido]pyridin-3-yl}cyclohexanecarboxylic acid ethyl ester; MH⁺412.00, RT 3.92 min.

trans-4-{4-[3-(5-Chloromethoxy-phenyl)ureido]-phenyl}cyclohexanecarboxylic acid isopropyl ester; MH⁺445.07, RT 4.54 min.

### Intermediate 39: trans-4-{4-[3-(2-Methoxy-5-methylphenyl)ureido]phenyl}cyclohexane-carboxylic acid

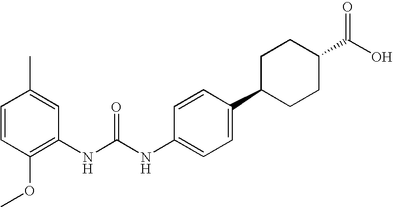

To a stirred solution of trans-4-{4-[3-(2-methoxy-5-methylphenyl)ureido]phenyl}-cyclohexanecarboxylic acid isopropyl ester (2.3 mmol) in THF/methanol (10 mL, 1:1) was added sodium hydroxide (2M, 9.2 mmol). After stirring overnight the mixture was partitioned between diethyl ether and water. The aqueous layer was separated, acidified with concentrated hydrochloric acid and extracted three times with ethyl aceate. These combined extract were dried (MgSO₄) and concentrated to afford the title compound. MH⁺383.08, RT 3.51 min.

### Intermediate 40

trans-4-(4-Nitrophenyl)cyclohexanecarboxylic acid was prepared in a similar manner to Intermediate 39, RT 4.24 min.

### Intermediate 41: trans 4-(4-Nitrophenyl)cyclohexanol

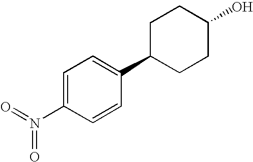

A solution of 4-(4-nitrophenyl)cyclohexanone (4.56 mmol) and pentafluorophenol (4.56 mmol) in THF (50 mL) was cooled to 0° C. and sodium borohydride (36.49 mmol) added portionwise over 5 min. After 15 min methanol was carefully added until effervescence had ceased and the mixture poured into 2M hydrochloric acid. The mixture was extracted three times with ethyl aceate and the combined extracts washed with sodium hydrogen carbonate and brine. The solution was dried (MgSO₄), concentrated and purified via flash chromatography to afford the title compound. RT 3.07 min.

### Intermediate 42: trans-[4-(4-Nitrophenyl)cyclohexyloxy]acetic acid methyl ester

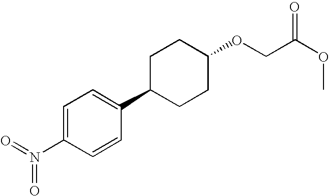

To a solution of trans 4-(4-nitrophenyl)cyclohexanol (1.13 mmol) in THF under argon atmosphere was added sodium hydride (2.26 mmol). After effervescence had ceased iodoacetic acid sodium salt (1.13 mmol) was added. After stirring for 3 days the mixture was partitioned between sodium hydroxide (0.5M) and diethyl ether. The aqueous layer was separated, acidified with concentrated hydrochloric acid and extracted three times with ethyl aceate. The combined organic extracts were dried (MgSO₄) concentrated and redissolved in DCM (5 mL) containing a few drops of methanol. Trimethylsilyldiazomethane (2M, hexane) was added in 0.25 mL aliquots until TLC indicated complete consumption of starting material. Acetic acid was added, and the reaction mixture diluted with ethyl acetate and washed with 1M sodium hydroxide, 1M hydrochloric acid and brine. The solution was dried and purified by flash chromatography to afford the title compound. RT 3.62 min.

### Intermediate 43: trans-[4-(4-Aminophenyl)cyclohexyloxy]acetic acid methyl ester

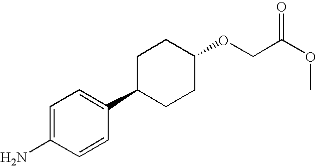

A suspension of trans-[4-(4-nitrophenyl)cyclohexyloxy]acetic acid methyl ester (0.21 g) and palladium on carbon (2 mg) in ethanol (1 mL) was stirred rapidly under hydrogen atmosphere for 3 h. The mixture was filtered through celite and concentrated to afford the title compound. MH⁺264.05, RT 2.14 min.

### Intermediate 44: trans-2-[(4-{4-[3-(2-Methoxy-5-ethylphenyl)ureido]phenyl}cyclohexane-carbonyl)amino]-2-methylpropionic acid methyl ester

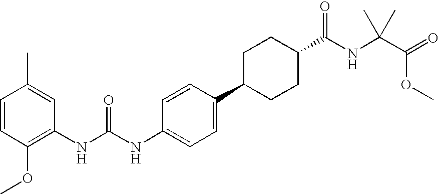

To a solution of trans-4-{4-[3-(2-methoxy-5-methyl-phenyl)ureido]phenyl}-cyclohexanecarboxylic acid (0.1 mmol) in DMF (1 mL) was added 1-(3-dimethylaminopropyl)-3-ethylcarbodiimide hydrochloride (0.2 mmol), 1-hydroxybenzotriazole (0.2 mmol) and triethylamine (0.41 mmol). After shaking for 20 min, 2-amino-2-methylpropionic acid methyl ester (0.2 mmol) was added and shaking continued overnight. The mixture was diluted with ethyl aceate and washed twice with water and twice with sodium carbonate. The solution was then washed with brine, dried and concentrated to obtain the title compound. MH⁺482.10, RT 3.64 min.

### Intermediates 45-52

The following intermediates were prepared by an analogous method to Intermediate 44:

trans-(S)-1-(4-{4-[3-(2-Methoxy-5-methylphenyl)ureido]phenyl}cyclohexane-carbonyl)pyrrolidine-2-carboxylic acid methyl ester; MH⁺494.11, RT 3.76 min.

trans-(R)-1-(4-{4-[3-(2-Methoxy-5-methylphenyl)ureido]-phenyl}cyclohexane-carbonyl)pyrrolidine-2-carboxylic acid methyl ester; MH⁺494.11, RT 3.65 min.

trans-(S)-1-[3-(4-{4-[3-(2-Methoxy-5-methylphenyl)ureido]phenyl}cyclohexyl)-propionyl]pyrrolidine-2-carboxylic acid methyl ester; MH⁺522.13, RT 3.99 min.

trans-(R)-1-[3-(4-{4-[3-(2-Methoxy-5-methylphenyl)ureido]phenyl}cyclohexyl)-propionyl]pyrrolidine-2-carboxylic acid methyl ester; MH⁺522.11, RT 3.90 min.

trans-[(4-{4-[3-(2-Methoxy-5-methylphenyl)ureido]phenyl}-cyclohexanecarbonyl)amino]acetic acid methyl ester; MH⁺454.15, RT 3.56 min.

trans-(S)-2-[(4-{4-[3-(2-Methoxy-5-methylphenyl)ureido]phenyl}cyclohexane-carbonyl)amino]-3-methylbutyric acid methyl ester; MH⁺496.17, RT 3.79 min.

trans-(S)-3-Hydroxy-2-[(4-{4-[3-(2-methoxy-5-methylphenyl)ureido]phenyl}-cyclohexanecarbonyl)amino]propionic acid methyl ester; MH⁺484.16, RT 3.37 min.

trans-(R)-3-Hydroxy-2-[(4-{4-[3-(2-methoxy-5-methylphenyl)ureido]phenyl}-cyclohexanecarbonyl)amino]propionic acid methyl ester; MH⁺484.15, RT 3.32 min.

It is to be appreciated that some of the compounds defined above are both examples of compounds of formula (I) and intermediates of use for the preparation of further compounds of formula (I), particularly via functional group conversion of the moiety —YC(O)Z.

### General Synthetic Methods

General synthetic methods, A to E, for the Examples are illustrated below.

### General Synthetic Method A

### Synthesis of trans-(4-{4-[3-(2-methoxy-5-methylphenyl)ureido]phenyl}cyclohexyl)acetic acid ethyl ester

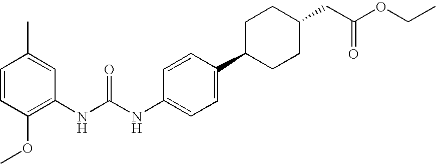

A mixture of trans-[4-(4-aminophenyl)cyclohexyl]acetic acid ethyl ester (0.19 mmol) and 2-isocyanato-1-methoxy-4-methylbenzene (0.21 mmol) was shaken in THF overnight. After this time, MP-isocyante and MP-trisamine were added and shaking continued for a further 5 h. The mixture was filtered and concentrated to obtain the title compound. MH⁺3.90, RT 425.06 min.

### General Synthetic Method B

### Synthesis of trans-(4-{4-[3-(2-methoxy-5-methylphenyl)ureido]phenyl}cyclohexyl)acetic acid

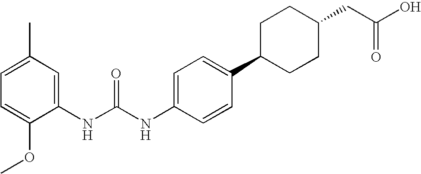

Trans-(4-{4-[3-(2-methoxy-5-methylphenyl)ureido]phenyl}cyclohexyl)acetic acid ethyl ester was dissolved in a 2:1 mixture of THF/MeOH. Sodium hydroxide (2M) was added and stirring continued until complete conversion to product was observed. The reaction mixture was diluted with methanol, and Dowex 50WX2-400 resin added to acidify the solution. The mixture was filtered and concentrated to obtain the title compound as a gum. MH⁺397.06, RT 3.74 min.

### General Synthetic Method C

### Synthesis of trans-1-{4-[4-((S)-2-hydroxymethylpyrrolidine-1-carbonyl)-cyclohexyl]phenyl}-3-(2-methoxy-5-methylphenyl)urea

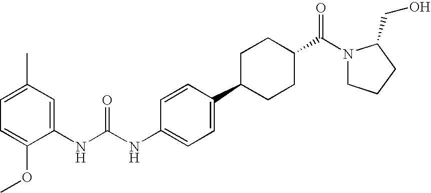

A flask was charged with trans-4-{4-[3-(2-methoxy-5-methylphenyl)ureido]pheny}-cyclohexanecarboxylic acid (0.26 mmol), 1-(3-dimethylaminopropyl)-3-ethylcarbodiimide hydrochloride (0.52 mmol), 1-hydroxybenzotriazole (0.52 mmol) and DMF (3 mL) added, followed by triethylamine (1.05 mmol). After shaking for 30 min, (R)-prolinol (0.52 mmol) was added and shaking continued for 16 h. The reaction mixture was diluted with DCM and water and shaken. The mixture was poured onto a hydrophobic frit and the DCM layer separated. The process was repeated, with the DCM layer being further shaken with water and saturated sodium carbonate solution. The DCM layer was then concentrated to afford the title compound. MH⁺466.13, RT 3.56 min.

### General Synthetic Method D

### Synthesis of trans-4-{4-[(2,3-Dihydroindole-1-carbonyl)amino]phenyl}-cyclohexanecarboxylic acid

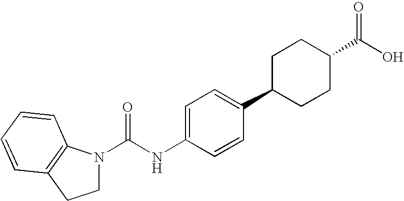

To a solution of triphosgene (0.26 g) in DCM (6 mL) under argon at 0° C. was added a solution of trans-4-(4-aminophenyl)cyclohexanecarboxylic acid isopropyl ester (0.6 g) in DCM (3 mL). After stirring for 10 min triethylamine (0.35 mg) was added. The reaction was stirred for a further 1 h and allowed to warm to room temperature over this time. The reaction mixture was split into three even aliquots, to one of which was added indoline (0.109 mg). The mixture agitated on a flatbed shaker for 72 h after which MP-isocyante and MP-trisamine were added and shaking continued for a further 5 h. The mixture was then filtered and concentrated. The resulting oil was triturated with diethyl ether and isopropanol to afford an off-white solid. This material was dissolved in a mixture of THF/methanol and sodium hydroxide (2M) added. After hydrolysis was complete, the mixture was diluted with water and extracted with ethyl acetate. The aqueous layer was separated, acidified by addition of 2M hydrochloric acid and extracted three times with ethyl acetate. The combined organic extracts were washed with brine and dried (MgSO₄), filtered and concentrated to obtain the title compound. MH⁺365.23, RT 3.50 min.

### General Synthetic Method E

### Synthesis of trans-(R)-1-(4-{4-[3-(2-methoxy-5-methylphenyl)ureido]phenyl}cyclohexane carbonyl)pyrrolidine-2-carboxylic acid

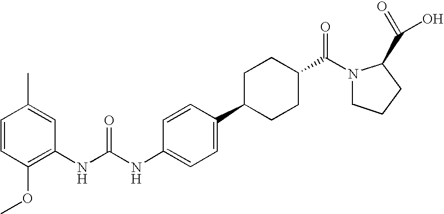

To a solution of trans-(R)-1-(4-{4-[3-(2-methoxy-5-methylphenyl)ureido]phenyl}-cyclohexanecarbonyl)pyrrolidine-2-carboxylic acid methyl ester (0.05 g) in THF (1 mL) was added a solution of lithium hydroxide (0.08 g) in water (1 mL). After shaking overnight the mixture was diluted with methanol and acidified with Dowex 50WX2-400 resin. The mixture was filtered and concentrated. The resulting residue was triturated with diethyl ether, filtered and dried to obtain the title compound. MH⁺480.11, RT 3.39 min.

The following compounds were prepared using the intermediates and general synthetic methods A-E described above or by the methods described in the Intermediates as indicated.

Retention times marked with an asterix were determined using LCMS method 2. In all other cases LCMS method 1 was employed.

| | | | General | | |
|---|---|---|---|---|---|
| | | | Synthetic | | |
| Ex | Structure | Name | Method | RT | m/z |
| 1 | 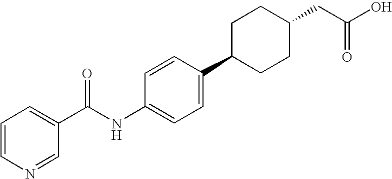 | Trans-(4-{4- [(Pyridine-3- carbonyl)amino]- phenyl}cyclohexyl)- acetic acid | B | 2.89 | 338.99 |
| 2 | 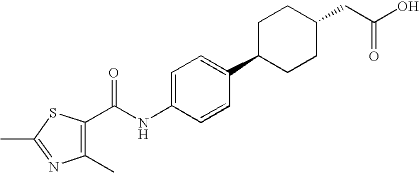 | Trans-(4-{4-[(2,4- Dimethylthiazole-5- carbonyl)amino]- phenyl}cyclohexyl)- acetic acid | B | 3.11 | 372.98 |
| 3 | 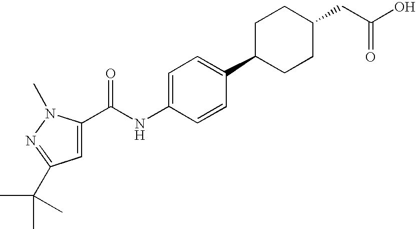 | Trans-(4-{4-[(5-tert- Butyl-2-methyl-2H- pyrazole-3-carbonyl)- amino]phenyl}- cyclohexyl)acetic acid | B | 3.31 | 398.08 |
| 4 | 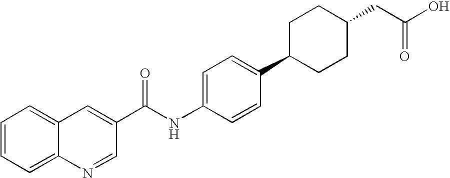 | Trans-(4-{4- [(Quinoline-3- carbonyl)amino]- phenyl}cyclohexyl)- acetic acid | B | 3.07 | 388.99 |
| 5 | 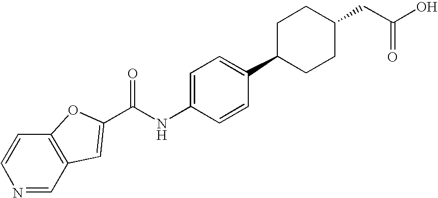 | Trans-(4-{4- [(Furo[3,2-c]- pyridine-2- carbonyl)amino]- phenyl}cyclohexyl)- acetic acid | B | 2.74 | 378.99 |
| 6 | 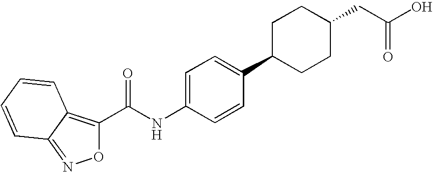 | Trans-(4-{4- [(Benzo[c]isoxazole- 3-carbonyl)amino]- phenyl}cyclohexyl)- acetic acid | B | 3.29 | 379.01 |
| 7 | 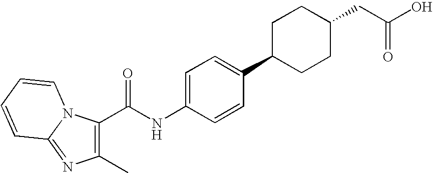 | Trans-(4-{4-[(2- Methylimidazol[1,2- a]pyridine-3- carbonyl)amino]- phenyl}cyclohexyl)- acetic acid | B | 2.64 | 392.03 |
| 8 | 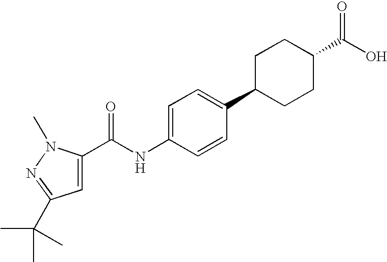 | Trans-4-{4-[(5-tert- Butyl-2-methyl-2H- pyrazole-3-carbonyl)- amino]phenyl}- cylohexane- carboxylic acid | B | 3.52 | 384.1 |
| 9 | 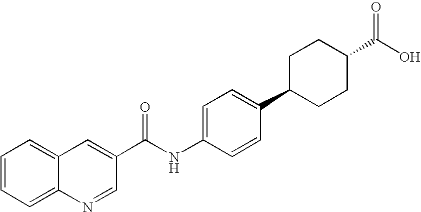 | Trans-4-{4- [(Quinoline-3- carbonyl)amino]- phenyl}- cyclohexane- carboxylic acid | B | 3.14 | 375.03 |
| 10 | 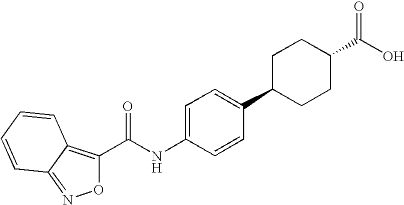 | Trans-4-{4- [(Benzo[c]isoxazole- 3-carbonyl)amino]- phenyl}- cyclohexane- carboxylic acid | See Inter- mediate 15 | 3.45 | 365.06 |
| 11 | 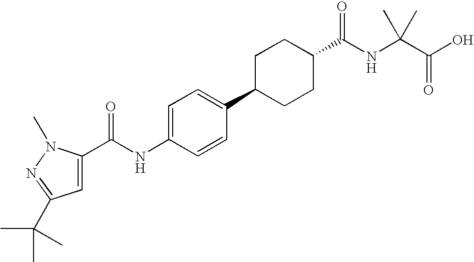 | Trans-2-[(4-{4-[(5- tert-Butyl-2-methyl- 2H-pyrazole-3- carbonyl)amino]- phenyl}- cyclohexane- carbonyl)amino]-2- methylpropionic acid | B | 3.44 | 469.09 |
| 12 | 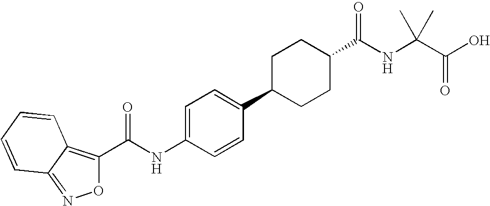 | Trans-2-[(4-{4- [(Benzo[c]isoxazole- 3-carbonyl)amino]- phenyl}- cyclohexane- carbonyl)amino]-2- methylpropionic acid | B | 3.31 | 450.07 |
| 13 | 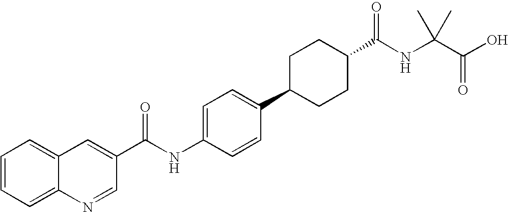 | Trans-2-Methyl-2- [(4-{4-[(quinoline-3- carbonyl)amino]- phenyl}- cyclohexane- carbonyl)amino]- propionic acid | B | 3.15 | 460.07 |
| 14 | 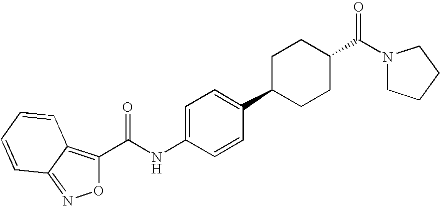 | Trans- Benzo[c]isoxazole-3- carboxylic acid {4- [4-(pyrrolidine-1- carbonyl)- cyclohexyl]phenyl}- amide | C | 3.67 | 418.10 |
| 15 | 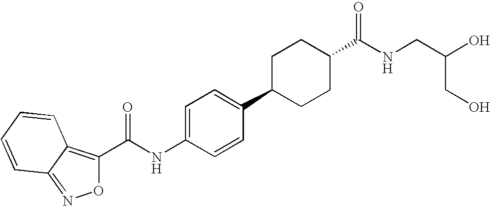 | Trans- Benzo[c]isoxazole-3- carboxylic acid {4- [4-(2,3-dihydroxy- propylcarbamoyl)- cyclohexyl]phenyl}- amide | C | 3.12 | 438.09 |
| 16 | 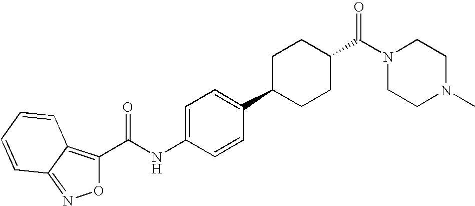 | Trans- Benzo[c]isoxazole-3- carboxylic acid {4- [4-(4-methyl- piperazine-1- carbonyl)- cyclohexyl]phenyl}- amide | C | 2.72 | 447.09 |
| 17 | 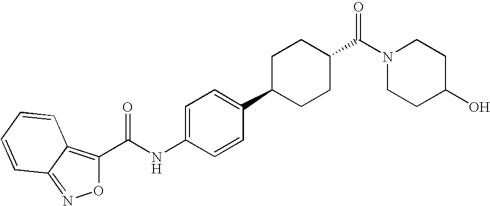 | Trans- Benzo[c]isoxazole-3- carboxylic acid {4- [4-(4-hydroxy- piperidine-1- carbonyl)- cyclohexyl]phenyl}- amide | C | 3.36 | 448.11 |
| 18 | 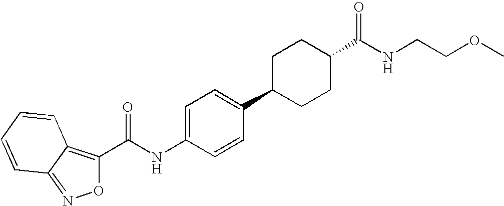 | Trans- Benzo[c]isoxazole-3- carboxylic acid {4- [4-(2-methoxy- ethylcarbamoyl)- cyclohexyl]phenyl}- amide | C | 3.45 | 422.08 |
| 19 | 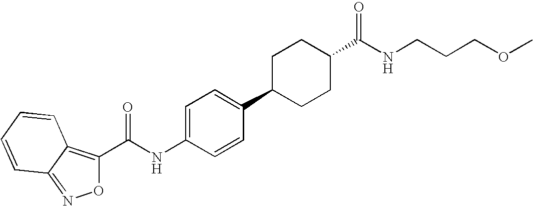 | Trans- Benzo[c]isoxazole-3- carboxylic acid {4- [4-(3-methoxy- propylcarbamoyl)- cyclohexyl]phenyl}- amide | C | 3.47 | 436.10 |
| 20 | 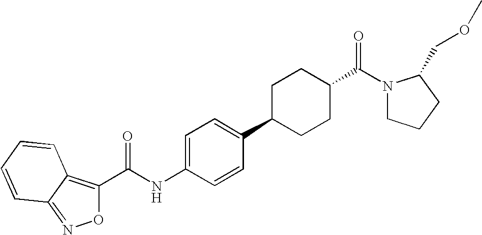 | Trans- Benzo[c]isoxazole-3- carboxylic acid ∴4- [4-((S)-2- methoxymethyl- pyrrolidine-1- carbonyl)- cyclohexyl]phenyl}- amide | C | 3.81 | 462.09 |
| 21 | 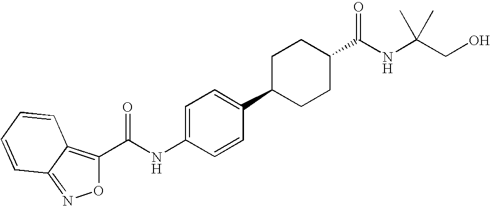 | Trans- Benzo[c]isoxazole-3- carboxylic acid {4- [4-(2-hydroxy-1,1- dimethyl- ethylcarbamoyl)- cyclohexyl]phenyl}- amide | C | 3.47 | 436.14 |
| 22 | 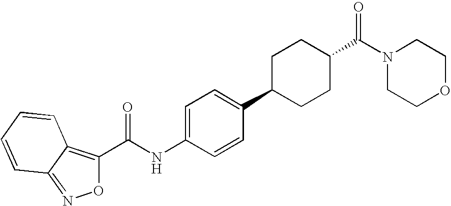 | Trans- Benzo[c]isoxazole-3- carboxylic acid {4- [4-(4-morpholine-4- carbonyl)- cyclohexyl]phenyl}- amide | C | 3.49 | 434.10 |
| 23 | 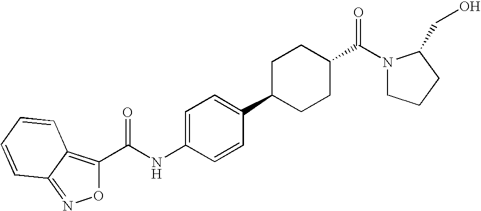 | Trans- Benzo[c]isoxazole-3- carboxylic acid {4- [4-((S)-2- hydroxymethyl- pyrrolidine-1- carbonyl)- cyclohexyl]phenyl}- amide | C | 3.44 | 448.08 |
| 24 | 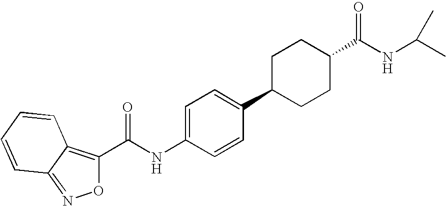 | Trans- Benzo[c]isoxazole-3- carboxylic acid {4-(4- isopropylcarbamoyl- cyclohexyl)phenyl]- amide | C | 3.62 | 406.12 |
| 25 | 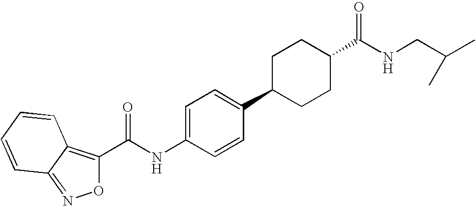 | Trans- Benzo[c]isoxazole-3- carboxylic acid [4-(4- isobutylcarbamoyl- cyclohexyl)phenyl]- amide | C | 3.76 | 420.13 |
| 26 | 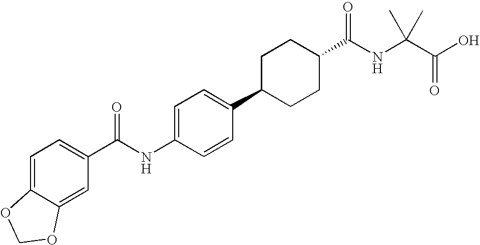 | Trans-2-({4-[4-(3- Benzo[1,3]dioxol-5- ylpropionylamino- phenyl]cyclohexane- carbonyl)amino]-2- methylpropionic acid | B | 1.60* | 481.31 |
| 27 | 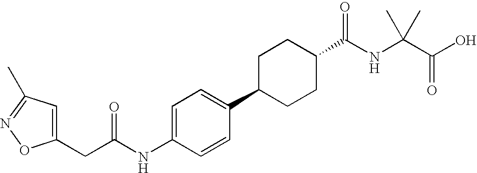 | Trans-2-Methyl-2- [(4-{4-[2-(3-methyl- isoxazol-5-yl)acetyl- amino]phenyl}- cyclohexane- carbonyl)amino]- propionic acid | B | 1.15* | 428.27 |
| 28 | 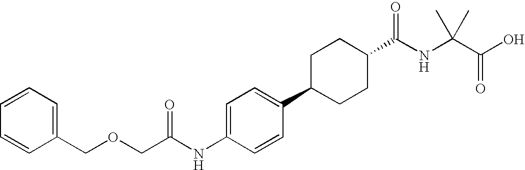 | Trans-2-({4-[4-(2- Benzyloxyacetyl- amino)phenyl]- cyclohexane- carbonyl}amino)-2- methylpropionic acid | B | 1.63* | 453.30 |
| 29 | 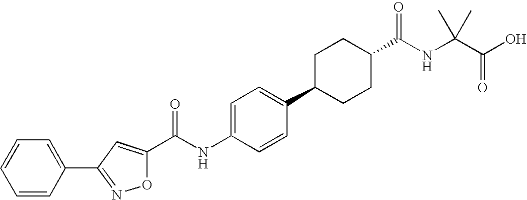 | Trans-2-Methyl-2- [4-{4-[(3-phenyl- isoxazole-5- carbonyl)amino]- phenyl}- cyclohexane- carbonyl)amino]- propionic acid | B | 1.93* | 476.30 |
| 30 | 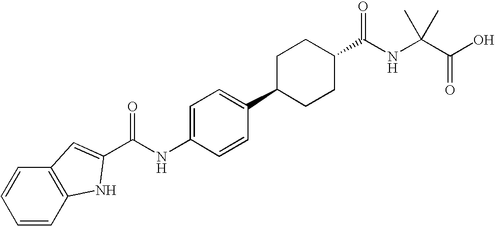 | Trans-2-[(4-{4-[(1H- Indole-2-carbonyl)- amino]phenyl}- cyclohexane- carbonyl)amino]-2- methylpropionic acid | B | 1.72* | 448.27 |
| 31 | 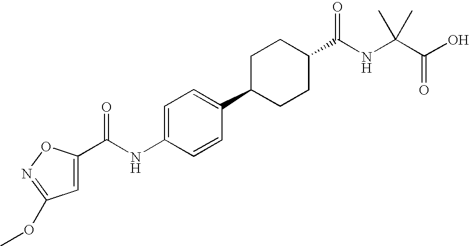 | Trans-2-[(4-{4-[(3- Methoxy-isoxazole- 5-carbonyl)amino]- phenyl}- cyclohexane- carbonyl)amino]-2- methylpropionic acid | B | 3.04 | 430.10 |
| 32 | 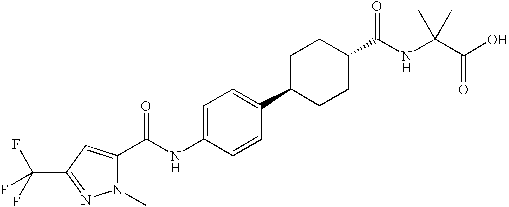 | Trans-2-Methyl-2- [(4-{4-[(2-methyl-5- trifluoromethyl-2H- pyrazole-3-carbonyl)- amino]phenyl}- cyclohexane- carbonyl)- amino]propionic acid | B | 1.72* | 481.36 |
| 33 | 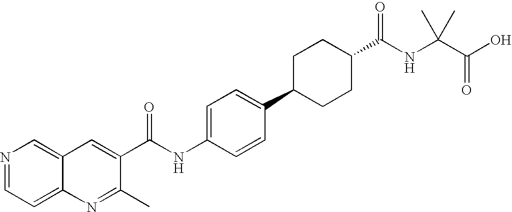 | Trans-2-Methyl-2- [(4-{4-[(2-methyl- [1,6]naphthyridine-3- carbonyl)amino]- phenyl}- cyclohexane- carbonyl)- amino]propionic acid | B | 2.76 | 475.09 |
| 34 | 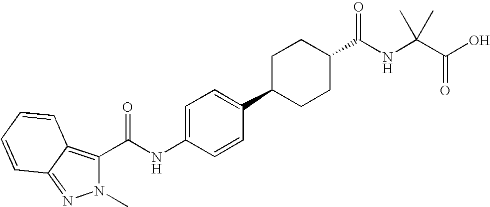 | Trans-2-Methyl-2- [(4-{4-[(2-methyl- 2H-indazole-3- carbonyl) amino]- phenyl}- cyclohexane- carbonyl)amino]- propionic acid | B | 1.55* | 463.30 |
| 35 | 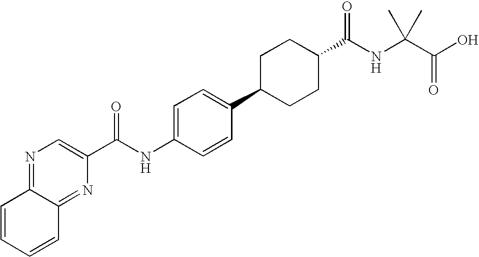 | Trans-2-Methyl-2- [(4-{4-[(quinoxaline- 2-carbonyl)amino]- phenyl}- cyclohexane- carbonyl)- amino]propionic acid | B | 1.67* | 461.36 |
| 36 | 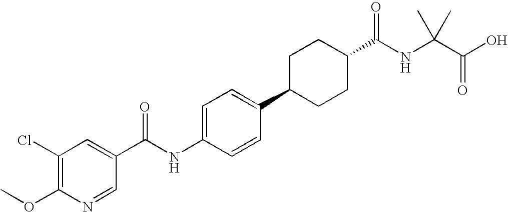 | Trans-2-[(4-{4-[(5- Chloro-6-methoxy- pyridine-3-carbonyl)- amino]phenyl}- cyclohexane- carbonyl)amino]-2- methylpropionic acid | B | 1.68* | 474.24 |
| 37 | 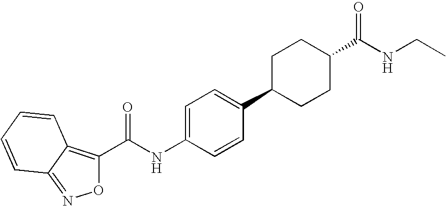 | Trans- Benzo[c]isoxazole-3- carboxylic acid [4-(4- ethylcarbamoyl- cyclohexyl)phenyl]- amide | C | 3.51 | 392.13 |
| 38 | 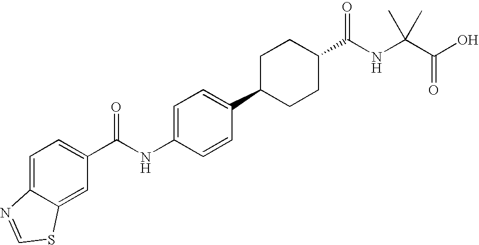 | Trans-2-[(4-{4- [(Benzothiazole-6- carbonyl)amino]- phenyl}- cyclohexane- carbonyl)amino]-2- methylpropionic acid | B | 3.15 | 466.07 |
| 39 | 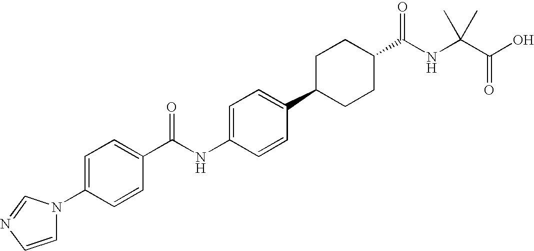 | Trans-2-({4-[4-(4- Imidazol-1-yl- benzoylamino)- phenyl]cyclohexane- carbonyl}amino)-2- methylpropionic acid | B | 1.43* | 475.36 |
| 40 | 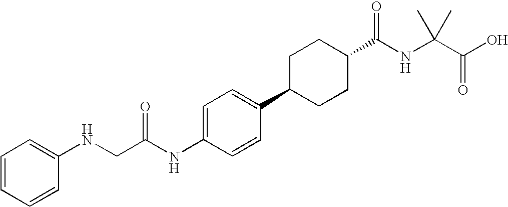 | Trans-2-Methyl-2- ({4-[4-(2-phenyl- aminoacetylamino)- phenyl]cyclohexane- carbonyl}amino)- propionic acid | B | 3.19 | 438.09 |
| 41 | 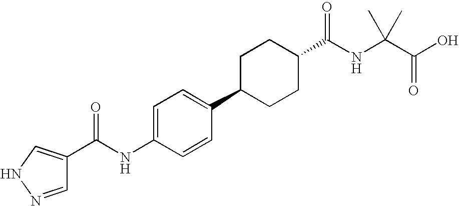 | Trans-2-Methyl-2- [(4-{4-[(1H-pyrazole- 4-carbonyl)amino]- phenyl}- cyclohexane- carbonyl)amino]- propionic acid | B | 2.67 | 399.11 |
| 42 | 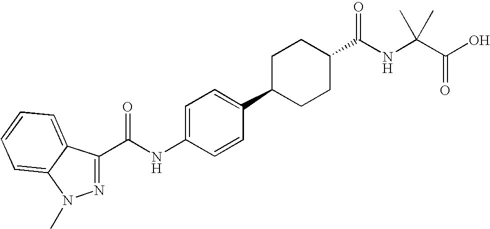 | Trans-2-Methyl-2- [(4-{4-[(1-methyl- 1H-indazole-3- carbonyl)amino]- phenyl}- cyclohexane- carbonyl)amino]- propionic acid | B | 1.77* | 463.32 |
| 43 | 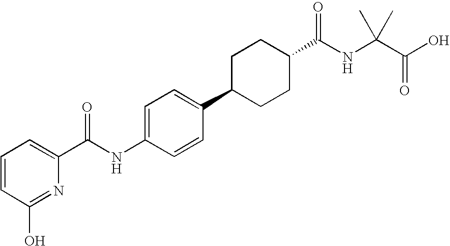 | Trans-2-[(4-{4-[(6- Hydroxypyridine-2- carbonyl)amino]- phenyl}- cyclohexane- carbonyl)amino]-2- methylpropionic acid | B | 0.83* | 426.24 |
| 44 | 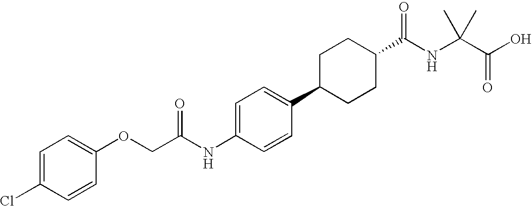 | Trans-2-[(4-{4-[2-(4- Chlorophenoxy)- acetylamino]- phenyl}- cyclohexane- carbonyl)amino]-2- methylpropionic acid | B | 1.87* | 473.25 |
| 45 | 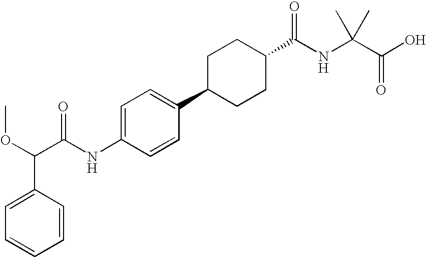 | Trans-2-({4-[4-(2- Methoxy-2-phenyl- acetylamino)phenyl]- cyclohexane- carbonyl}amino)-2- methylpropionic acid | B | 1.65* | 453.27 |
| 46 | 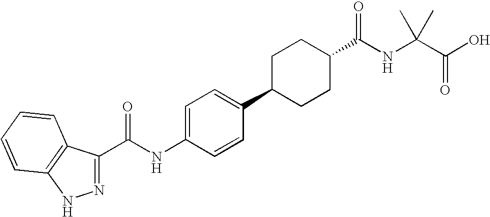 | Trans-2-[(4-{4-[(1H- Indazole-3-carbonyl)- amino]phenyl}- cyclohexane- carbonyl)amino]-2- methylpropionic acid | B | 1.62* | 449.26 |
| 47 | 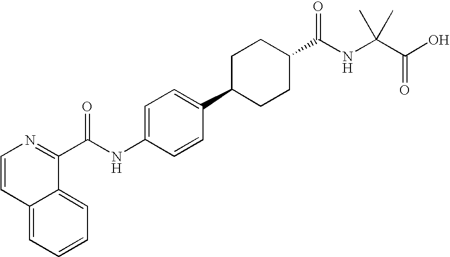 | Trans-2-[(4-{4- [(Isoquinoline-1- carbonyl)amino]- phenyl}- cyclohexane- carbonyl)amino]-2- methylpropionic acid | B | 1.80* | 460.27 |
| 48 | 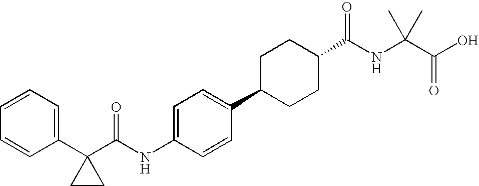 | Trans-2-Methyl-2- [(4-{4-[(1-phenyl- cyclopropane- carbonyl)amino]- phenyl}- cyclohexane- carbonyl)amino]- propionic acid | B | 1.82* | 449.24 |
| 49 | 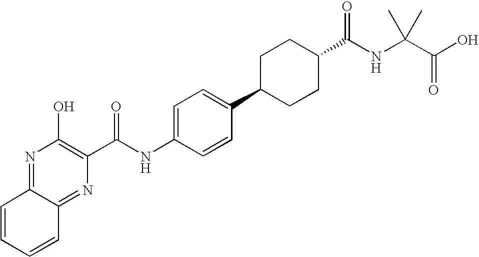 | Trans-2-[(4-{4-[(3- Hydroxy- quinoxaline-2- carbonyl)amino]- phenyl}- cyclohexane- carbonyl)amino]-2- methylpropionic acid | B | 0.97* | 477.16 |
| 50 | 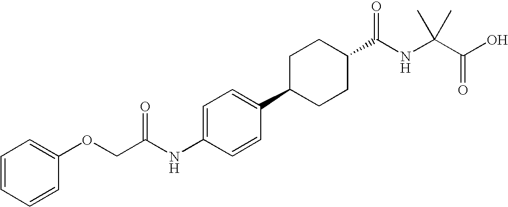 | Trans-2-Methyl-2- ({4-[4-(2-phenoxy- acetylamino)- phenyl]cyclohexane- carbonyl}amino)- propionic acid | B | 1.64* | 439.26 |
| 51 | 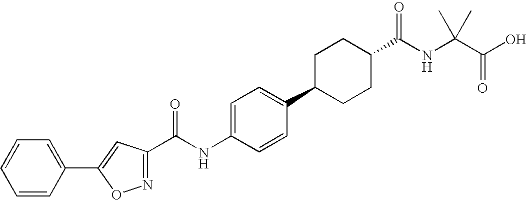 | Trans-2-Methyl-2- [(4-{4-[(5-phenyl- isoxazole-3- carbonyl)amino]- phenyl}- cyclohexane- carbonyl)amino]- propionic acid | B | 1.97* | 476.29 |
| 52 | 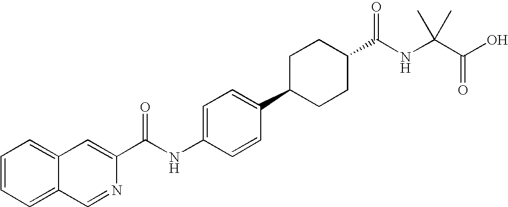 | Trans-2-[(4-{4- [(Isoquinoline-3- carbonyl)amino]- phenyl}- cyclohexane- carbonyl)amino]-2- methylpropionic acid | B | 1.87* | 460.28 |
| 53 | 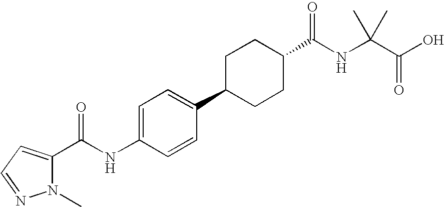 | Trans-2-Methyl-2- [(4-{4-[(2-methyl- 2H-pyrazole-3- carbonyl)amino]- phenyl}- cylohexane- carbonyl)amino]- propionic acid | B | 1.28* | 413.19 |
| 54 | 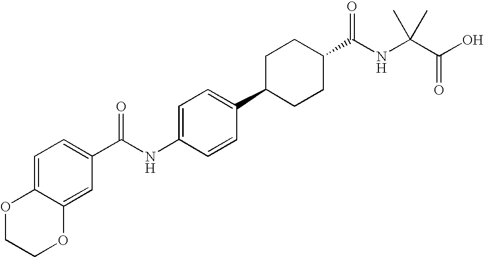 | Trans-2-[(4-{4-[(2,3- Dihydrobenzo[1,4]- dioxine-6-carbonyl)- amino]phenyl}- cyclohexane- carbonyl)amino]-2- methylpropionic acid | B | 1.65* | 467.30 |
| 55 | 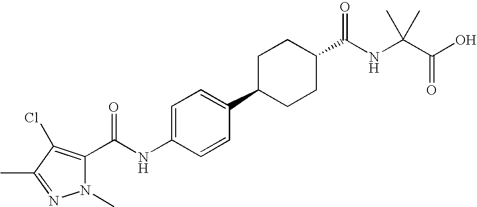 | Trans-2-[(4-{4-[(4- Chloro-2,5-dimethyl- 2H-pyrazole-3- carbonyl)amino]- phenyl}- cyclohexane- carbonyl)amino]-2- methylpropionic acid | B | 1.68* | 461.27 |
| 56 | 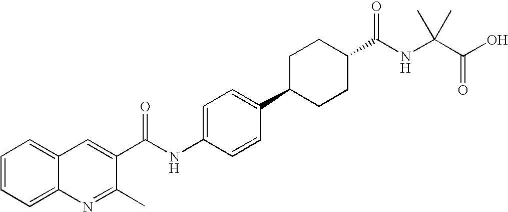 | Trans-2-Methyl-2- [(4-{4-[(2-methyl- quinoline-3- carbonyl)amino]- phenyl}- cyclohexane- carbonyl)- amino]propionic acid | B | 1.62* | 474.31 |
| 57 | 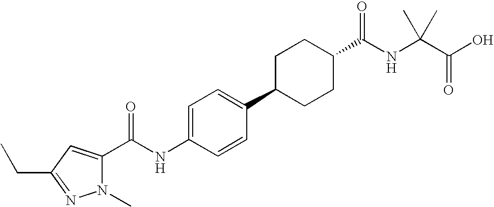 | Trans-2-[(4-{4-[(5- Ethyl-2H-pyrazole-3- carbonyl)amino]- phenyl}- cyclohexane- carbonyl)amino]-2- methylpropionic acid | B | 1.60* | 441.31 |
| 58 | 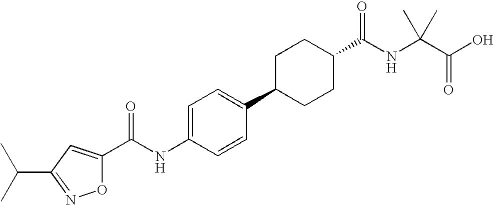 | Trans-2-[(4-{4-[(3- Isopropyl-isoxazole- 5-carbonyl)amino]- phenyl}- cyclohexane- carbonyl)amino]-2- methylpropionic acid | B | 1.75* | 442.30 |
| 59 | 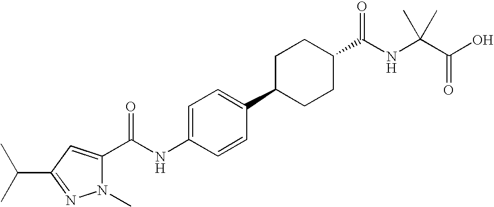 | Trans-2-[(4-{4-[(5- Isopropyl-2H- pyrazole-3-carbonyl)- amino]phenyl}- cyclohexane- carbonyl)amino]-2- methylpropionic acid | B | 1.77* | 455.33 |
| 60 | 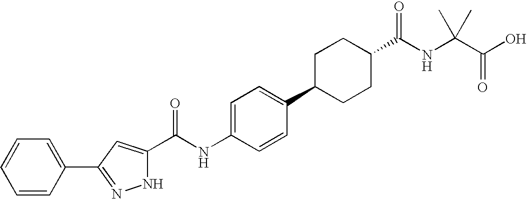 | Trans-2-Methyl-2- [(4-{4-[(5-phenyl- 2H-pyrazole-3- carbonyl)- amino]phenyl}- cyclohexane- carbonyl)amino]- propionic acid | B | 3.29 | 475.12 |
| 61 | 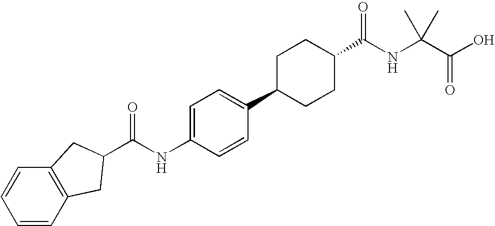 | Trans-2-[(4-{4- [(Indane-2- carbonyl)amino]- phenyl}cyclohexane- carbonyl)amino]-2- methylpropionic acid | B | 1.74* | 449.41 |
| 62 | 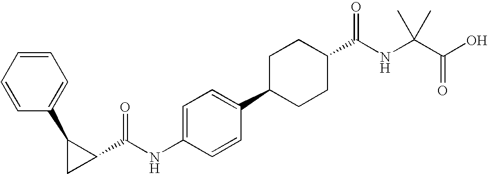 | Trans-2-Methyl-2- [(4-{4-[((1R,2R)-2- phenylcyclopropane- carbonyl)amino]- phenyl}cyclohexane- carbonyl)amino]- propionic acid | B | 1.77* | 449.45 |
| 63 | 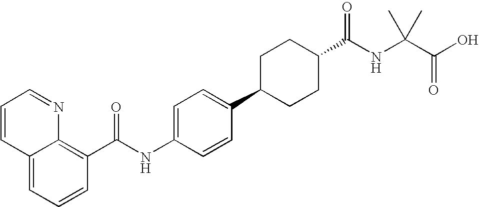 | Trans-2-Methyl-2- [(4-{4-[(quinoline-8- carbonyl)amino]- phenyl}cyclohexane- carbonyl)amino]- propionic acid | B | 1.80* | 460.44 |
| 64 | 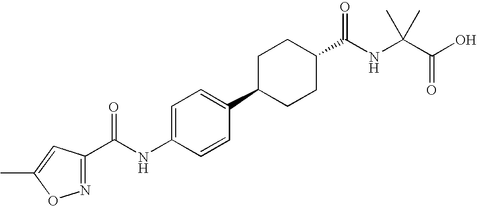 | Trans-2-Methyl-2- [(4-{4-[(5-methyl- isoxazole-3- carbonyl)amino]- phenyl}cyclohexane- carbonyl)amino]- propionic acid | B | 1.42* | 414.33 |
| 65 | 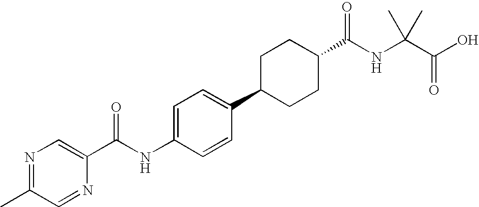 | Trans-2-Methyl-2- [(4-{4-[(5-methyl- pyrazine-2-carbonyl)- amino]phenyl}- cyclohexane- carbonyl)amino] propionic acid | B | 1.37* | 425.37 |
| 66 | 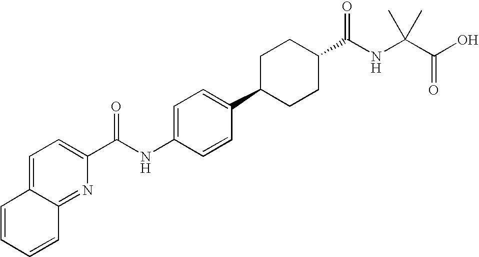 | Trans-2-Methyl-2- [(4-{4-[(quinoline-2- carbonyl)amino]- phenyl}cyclohexane- carbonyl)amino] propionic acid | B | 1.87* | 460.38 |
| 67 | 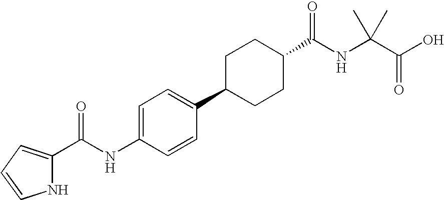 | Trans-2-Methyl-2- [(4-{4-[(1H-pyrrole- 2-carbonyl)amino]- phenyl}cyclohexane- carbonyl)amino] propionic acid | B | 1.32* | 398.32 |
| 68 | 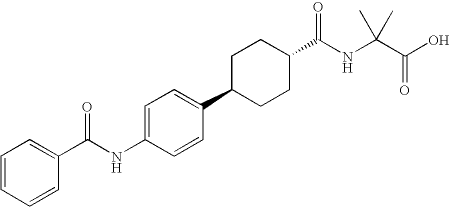 | Trans-2-{(4-(4- Benzoylamino- phenyl)cyclohexane- carbonyl]amino}-2- methylpropionic acid | B | 1.47* | 409.30 |
| 69 | 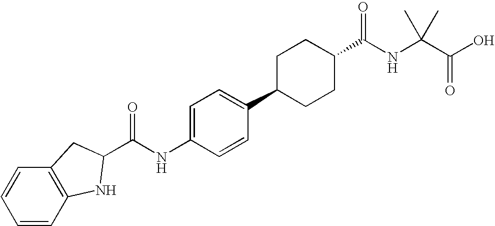 | Trans-2-[(4-{4-[(2,3- Dihydro-1H-indole- 2-carbonyl)amino]- phenyl}cyclohexane- carbonyl)amino]-2- methylpropionic acid | B | 3.14 | 450.10 |
| 70 | 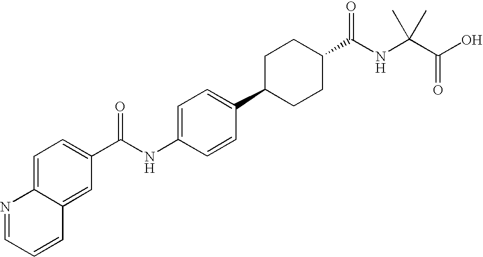 | Trans-2-Methyl-2- [(4-{4-[(quinoline-6- carbonyl)amino]- phenyl}cyclohexane- carbonyl)amino]- propionic acid | B | 1.49* | 460.27 |
| 71 | 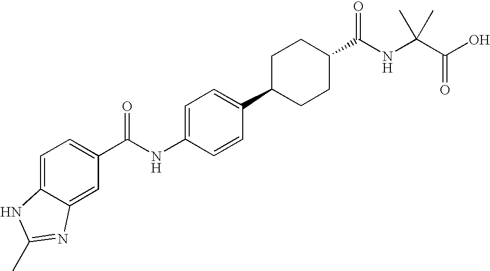 | Trans-2-Methyl-2- [(4-{4-[(2-methyl- 1H-benzoimidazole- 5-carbonyl)amino]- phenyl}cyclohexane- carbonyl)amino]- propionic acid | B | 1.28* | 463.30 |
| 72 | 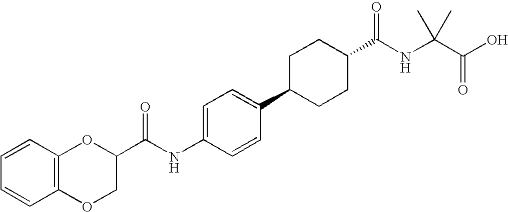 | Trans-2-[(4-{4-[(2,3- Dihydrobenzo[1,4]- dioxine-2-carbonyl)- amino]phenyl}- cyclohexane- carbonyl)amino]-2- methylpropionic acid | B | 3.34 | 467.11 |
| 73 | 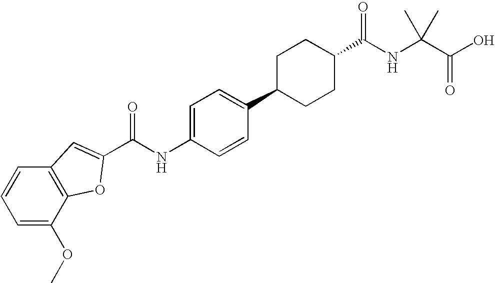 | Trans-2-[(4-{4-[(7- Methoxybenzofuran- 2-carbonyl)amino]- phenyl}cyclohexane- carbonyl)amino]-2- methylpropionic acid | B | 3.42 | 479.10 |
| 74 | 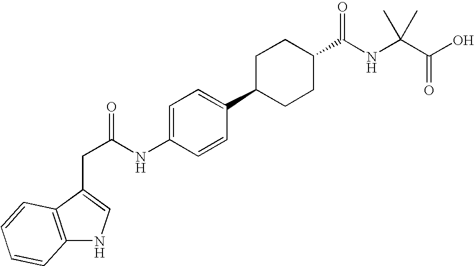 | Trans-2-({4-[4-(2- 1H-Indol-3-yl- acetylamino)phenyl]- cyclohexane- carbonyl}amino)-2- methylpropionic acid | B | 1.59* | 462.34 |
| 75 | 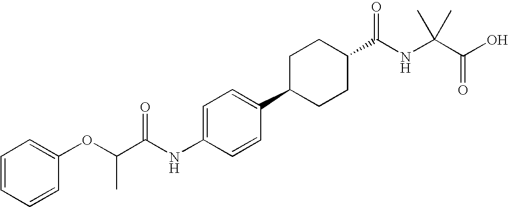 | Trans-2-Methyl-2- ({4-[4-(2-phenoxy- propionylamino)- phenyl]cyclohexane- carbonyl}amino)- propionic acid | B | 3.36 | 453.12 |
| 76 | 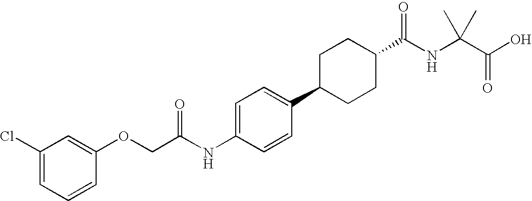 | Trans-2-[(4-{4-[2-(3- Chlorophenoxy)- acetylamino]- phenyl}cyclohexane- carbonyl)amino]-2- methylpropionic acid | B | 1.84* | 473.23 |
| 77 | 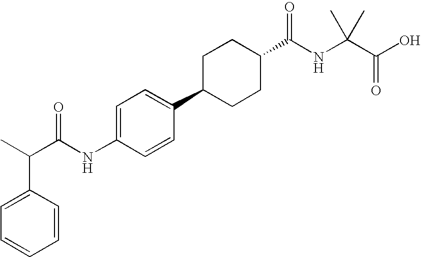 | Trans-2-Methyl-2- ({4-[4-(2-phenyl- propionylamino)- phenyl]cyclohexane- carbonyl}amino)- propionic acid | B | 1.67* | 437.28 |
| 78 | 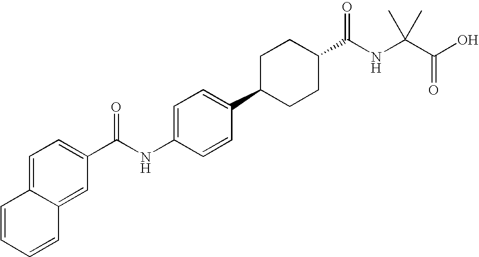 | Trans-2-Methyl-2- [(4-{4-[(naphthalene- 2-carbonyl)amino]- phenyl}cyclohexane- carbonyl)amino]- propionic acid | B | 3.51 | 459.11 |
| 79 | 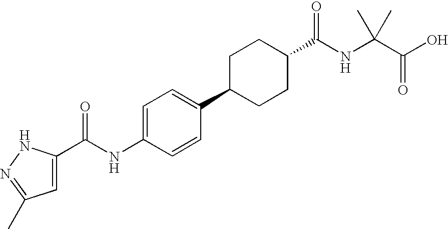 | Trans-2-Methyl-2- [(4-{4-[(5-methyl- 2H-pyrazole-3- carbonyl)amino]- phenyl}cyclohexane- carbonyl)amino]- propionic acid | B | 1.30* | 413.22 |
| 80 | 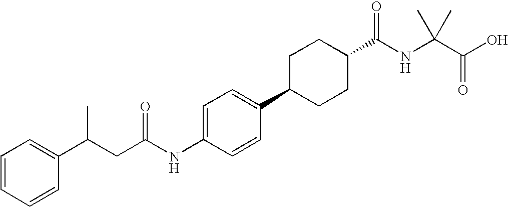 | Trans-2-Methyl-2- ({4-[4-(3-phenyl- butyrylamino)- phenyl]cyclohexane- carbonyl}amino)- propionic acid | B | 1.74* | 451.31 |
| 81 | 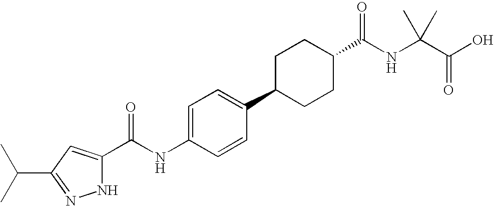 | Trans-2-[(4-{4-[(5- Isopropyl-2H- pyrazole-3-carbonyl)- amino]phenyl}- cyclohexane- carbonyl)amino]-2- methylpropionic acid | B | 3.14 | 441.11 |
| 82 | 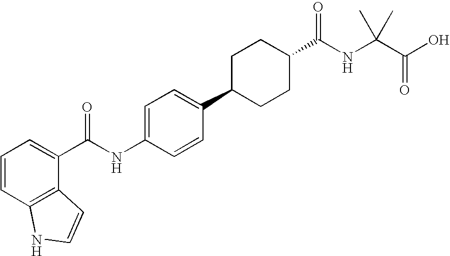 | Trans-2-[(4-{4-[(1H- Indole-4-carbonyl)- amino]phenyl}- cyclohexane- carbonyl)amino]-2- methylpropionic acid | B | 1.46* | 448.28 |
| 83 | 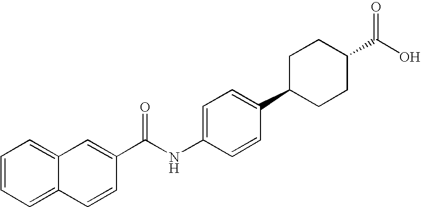 | Trans-4-{4- [(Naphthalene-2- carbonyl)amino]- phenyl}- cyclohexane- carboxylic acid | B | 3.62 | 374.08 |
| 84 | 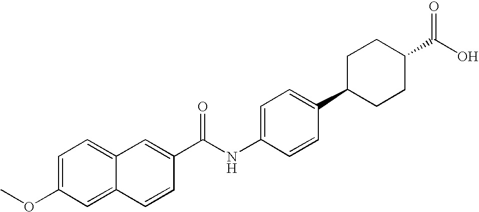 | Trans-4-{4-[(6- Methoxynaphthalene- 2-carbonyl)amino]- phenyl}cyclohexane- carboxylic acid | B | 3.65 | 404.09 |
| 85 | 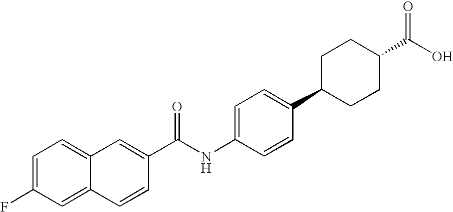 | Trans-4-{4-[(6- Fluoronaphthalene-2- carbonyl)amino]- phenyl}cyclohexane- carboxylic acid | B | 3.72 | 392.07 |
| 86 | 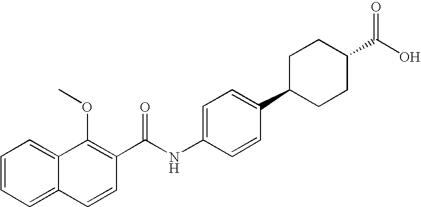 | Trans-4-{4-[(1- Methoxynaphthalene- 2-carbonyl)amino]- phenyl}cyclohexane- carboxylic acid | B | 3.77 | 404.08 |
| 87 | 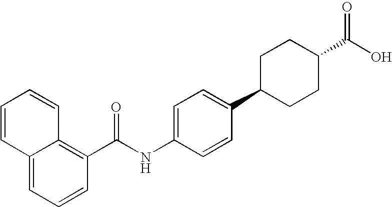 | Trans-4-{4- [(Naphthalene-2- carbonyl)amino]- phenyl}cyclohexane- carboxylic acid | B | 3.54 | 374.08 |
| 88 | 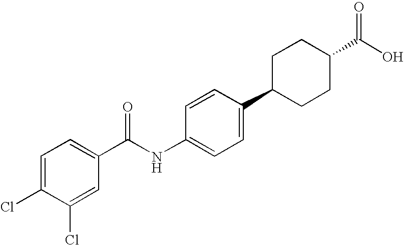 | Trans-4-[4-(3,4- Dichlorobenzoyl- amino)phenyl]- cyclohexane- carboxylic acid | See Inter- mediate 18 | 3.82 | 391.91 |
| 89 | 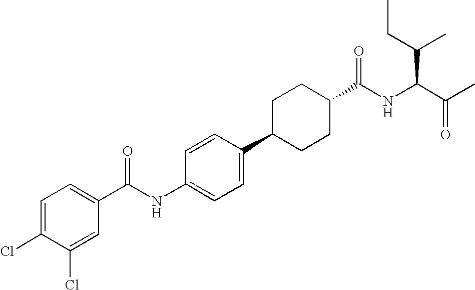 | Trans-(S)-2-({4-[4- (3,4-Dichloro- benzoylamino)- phenyl]cyclohexane- carbonyl}amino)-3- methylpentanoic acid methyl ester | C | 4.16 | 519.07 |
| 90 | 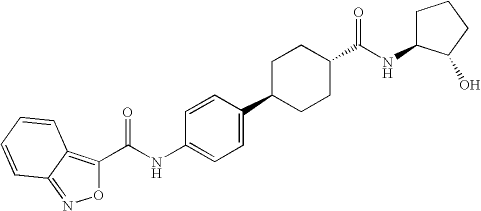 | Trans- Benzo[c]isoxazole-3- carboxylic acid {4- [4-((1S,2S)-2- hydroxycyclopentyl- carbamoyl)cyclo- hexyl]phenyl}amide | C | 3.40 | 448.09 |
| 91 | 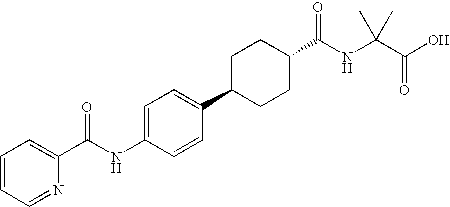 | Trans-2-Methyl-2- [(4-{4-[(pyridine-2- carbonyl)amino]- phenyl}cyclohexane- carbonyl)amino]- propionic acid | B | 1.53* | 410.31 |
| 92 | 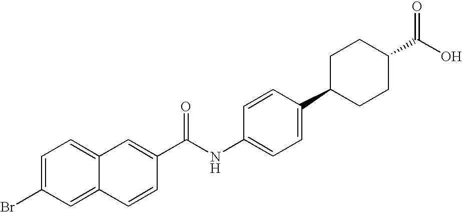 | Trans-4-{4-[(6- Bromonaphthalene-2- carbonyl)amino]- phenyl}cyclohexane- carboxylic acid | B | 3.87 | 453.96 |
| 93 | 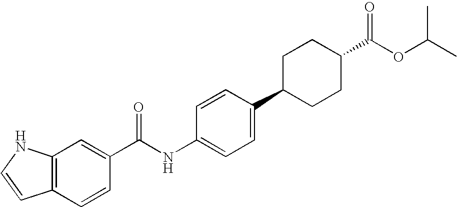 | Trans-4-{4-[(1H- Indole-6-carbonyl)- amino]phenyl}- cyclohexane- carboxylic acid isopropyl ester | C | 3.90 | 405.08 |
| 94 | 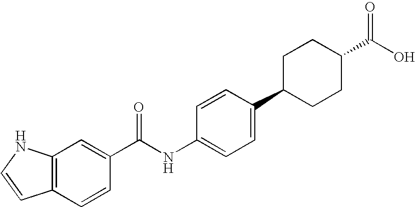 | Trans-4-{4-[(1H- Indole-6-carbonyl)- amino]phenyl}- cyclohexane- carboxylic acid | B | 3.32 | 363.15 |
| 95 | 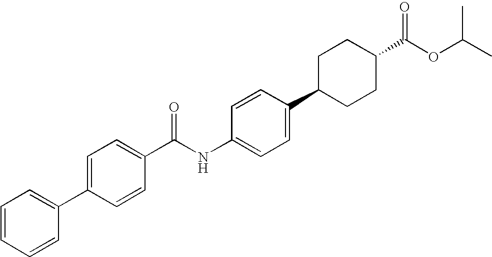 | Trans-4-{[(Biphenyl- 4-carbonyl)amino]- phenyl}cyclohexane- carboxylic acid isopropyl ester | C | 4.49 | 442.09 |
| 96 | 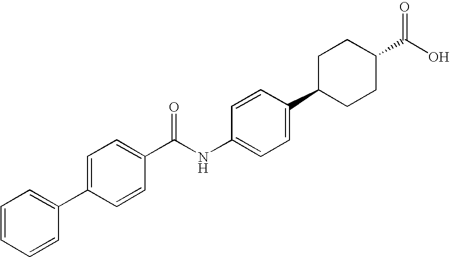 | Trans-4-{[(Biphenyl- 4-carbonyl)amino]- phenyl}cyclohexane- carboxylic acid | B | 3.79 | 400.07 |
| 97 | 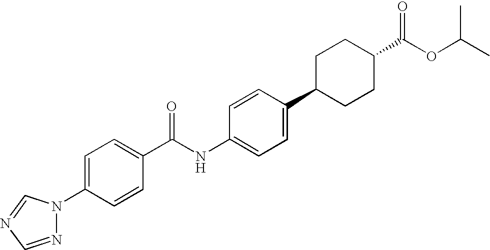 | Trans-4-[4-(4- [1,2,4]Triazol-1-yl- benzoylamino)- phenyl]cyclohexane- carboxylic acid isopropyl ester | C | 3.87 | 433.07 |
| 98 | 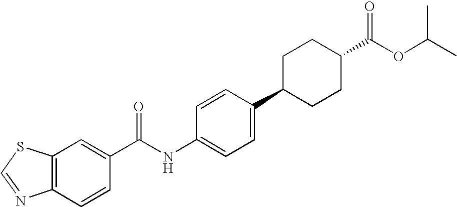 | Trans-4-{4- [(Benzothiazole-6- carbonyl)amino]- phenyl}cyclohexane- carboxylic acid isopropyl ester | C | 4.01 | 423.03 |
| 99 | 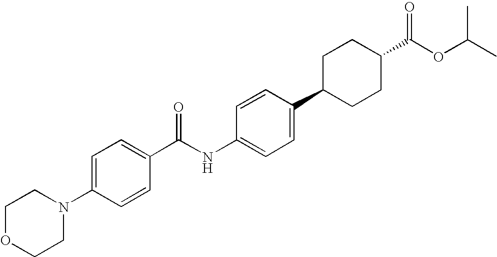 | Trans-4-[4-(4- Morpholino-4-yl- benzoylamino)- phenyl]cyclohexane- carboxylic acid isopropyl ester | C | 4.08 | 451.08 |
| 100 | 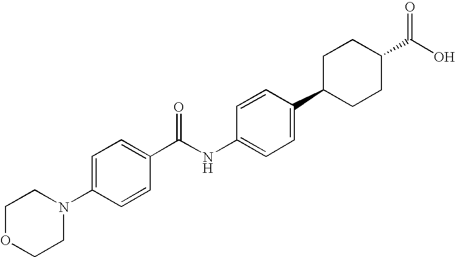 | Trans-4-[4-(4- Morpholino-4-yl- benzoylamino)- phenyl]cyclohexane- carboxylic acid | B | 3.27 | 409.06 |
| 101 | 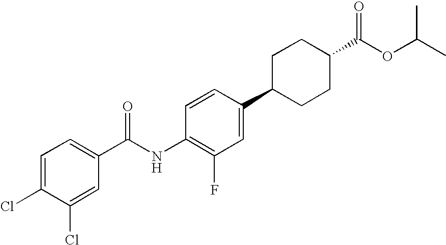 | Trans-4-[4-(3,4- Dichlorobenzoyl- amino)-3- fluorophenyl]- cyclohexane- carboxylic acid isopropyl ester | C | 4.36 | 451.98 |
| 102 | 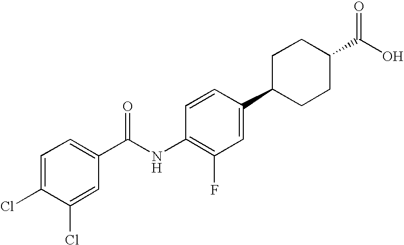 | Trans-4-[4-(3,4- Dichlorobenzoyl- amino)-3- fluorophenyl]- cyclohexane- carboxylic acid | B | 3.74 | 409.90 |
| 103 | 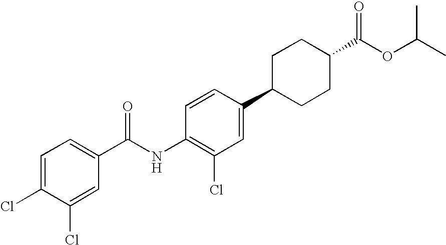 | Trans-4-[3-Chloro-4- (3,4-dichlorobenzoyl- amino)phenyl]- cyclohexane- carboxylic acid isopropyl acid | C | 4.61 | 467.93 |
| 104 | 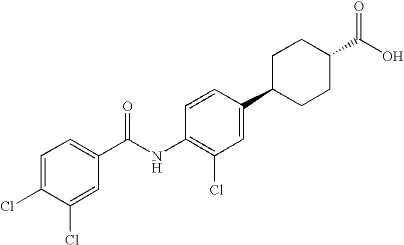 | Trans-4-[3-Chloro-4- (3,4-dichlorobenzoyl- amino)phenyl]- cyclohexane- carboxylic acid | B | 3.95 | 425.93 |
| 105 | 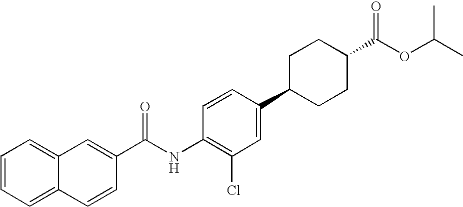 | Trans-4-{3-Chloro-4- [(naphthalene-2- carbonyl)amino]- phenyl}cyclohexane- carboxylic acid isopropyl ester | C | 4.55 | 450.04 |
| 106 | 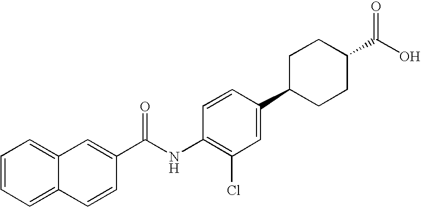 | Trans-4-{3-Chloro-4- [(naphthalene-2- carbonyl)amino]- phenyl}cyclohexane- carboxylic acid | B | 3.87 | 408.00 |
| 107 | 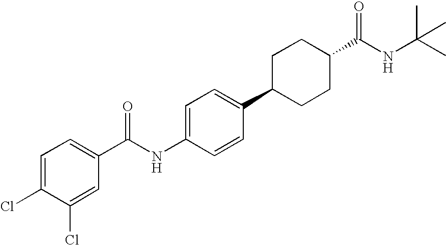 | Trans-4-[4-(4-tert- Butylcarbamoyl- cyclohexyl)phenyl]- 3,4-dichloro- benzamide | C | 4.14 | 447.01 |
| 108 | 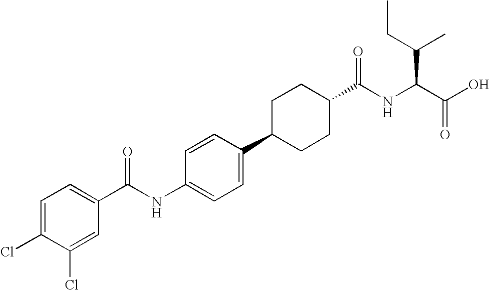 | Trans-(S)-2-({4-[4- (3,4-Dichloro- benzoylamino)- phenyl]cyclohexane- carbonyl}amino)-3- methylpentanoic acid | B | 3.81 | 505.07 |
| 109 | 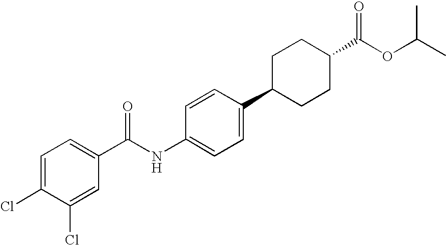 | Trans-4-[4-(3,4- Dichlorobenzoyl- amino)phenyl]- cyclohexane- carboxylic acid isopropyl ester | See Inter- mediate 18 | 4.41 | 433.99 |
| 110 | 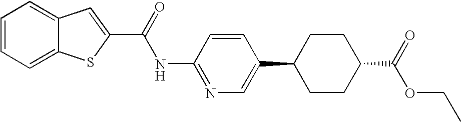 | Trans-4-{6- [(Benzo[b]thiophene- 2-carbonyl)amino]- pyridine-3-yl}- cyclohexane- carboxylic acid ethyl ester | C | 4.02 | 408.95 |
| 111 | 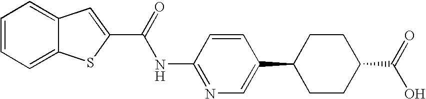 | Trans-4-{6- [(Benzo[b]thiophene- 2-carbonyl)-amino]- pyridine-3-yl}- cyclohexane- carboxylic acid | B | 3.39 | 380.93 |
| 112 | 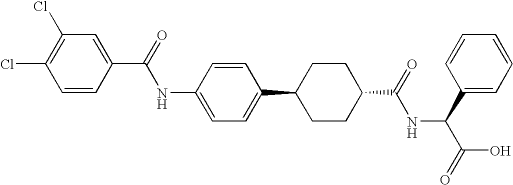 | Trans-(S)-({4-[4- (3,4-Dichloro- benzoylamino)- phenyl]cyclohexane- carbonyl}amino)- phenyl acetic acid | B | 3.90 | 524.91 |
| 113 | 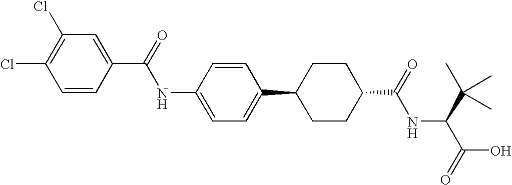 | Trans-(S)-({4-[4- (3,4-Dichloro- benzoylamino)- phenyl]cyclohexane- carbonyl}amino)-3,3- dimethyl butyric acid | B | 3.87 | 504.95 |
| 114 | 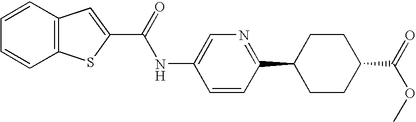 | Trans-4-{5- [(Benzo[b]thiophene- 2-carbonyl)amino]- pyridin-2-yl}- cyclohexane- carboxylic acid methyl ester | C | 3.19 | 394.88 |
| 115 | 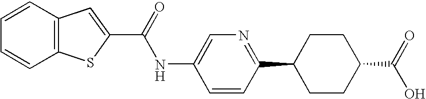 | Trans-4-{5- [(Benzo[b]thiophene- 2-carbonyl)amino]- pyridin-2-yl}- cyclohexane- carboxylic acid | B | 2.90 | 380.89 |
| 116 | 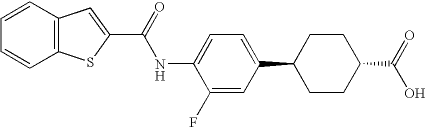 | Trans-4-{4- [(Benzo[b]thiophene- 2-carbonyl)amino]-3- fluorophenyl}- cyclohexane- carboxylic acid | B | 3.79 | 397.98 |
| 117 | 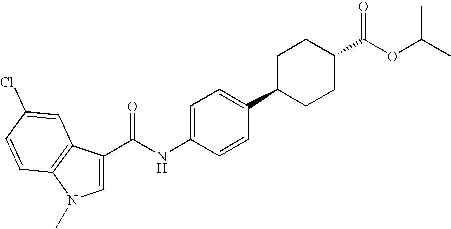 | Trans-4-{4-[(5- Chloro-1-methyl-1H- indole-3-carbonyl)- amino]phenyl}- cyclohexane- carboxylic acid isopropyl ester | C | 4.36 | 452.95 |
| 118 | 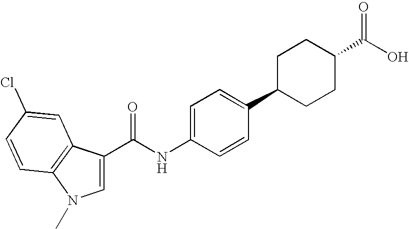 | Trans-4-{4-[(5- Chloro-1-methyl-1H- indole-3-carbonyl)- amino]phenyl}- cyclohexane- carboxylic acid | B | 3.69 | 409.35 |
| 119 | 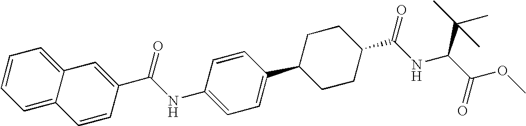 | Trans-(S)-3,3- Dimethyl-2-[(4-{4- [(naphthalene-2- carbonyl)amino]- phenyl}- cyclohexane- carbonyl)amino]- butyric acid methyl ester | C | 4.09 | 501.04 |
| 120 | 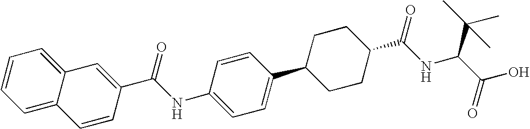 | Trans-(S)-3,3- Dimethyl-2-[(4-{4- [(naphthalene-2- carbonyl)amino]- phenyl}- cyclohexane- carbonyl)amino]- butyric acid | B | 3.81 | 487.04 |
| 121 | 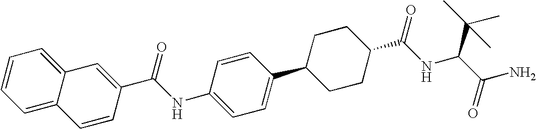 | Trans-Naphthalene- 2-carbocylic acid-{4- [4-((S)-1-carbamoyl- 2,2-dimethylpropyl- carbamoyl)- cyclohexyl]phenyl amide | C | 3.69 | 486.03 |
| 122 | 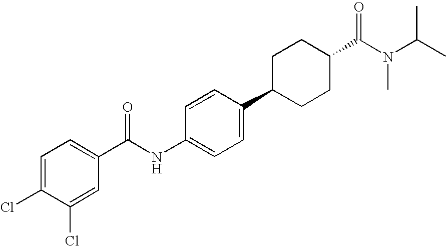 | Trans-3,4-Dichloro- N-{4-[4-(isopropyl- methylcarbamoyl)- cyclohexyl]phenyl}- benzamide | C | 4.09 | 446.92 |
| 123 | 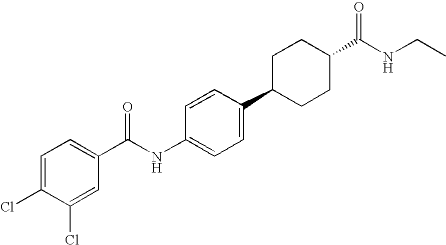 | Trans-3,4-Dichloro- N-[4-(4-ethyl- carbamoyl- cyclohexyl)phenyl]- benzamide | C | 3.81 | 420.98 |
| 124 | 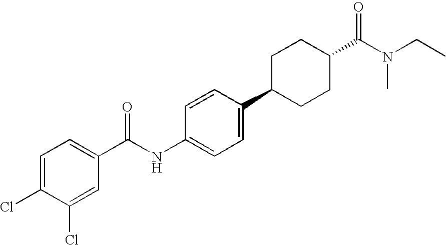 | Trans-3,4-Dichloro- N-{4-[4-(ethyl- methylcarbamoyl)- cyclohexyl]pehnyl}- benzamide | C | 3.99 | 432.98 |
| 125 | 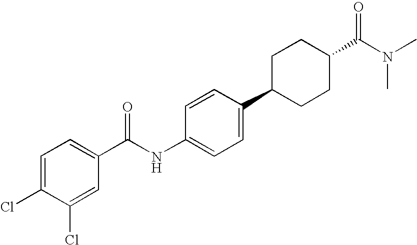 | Trans-3,4-Dichloro- N-[4-(4-(dimethyl- carbamoyl- cyclohexyl)phenyl]- benzamide | C | 3.84 | 418.94 |
| 126 | 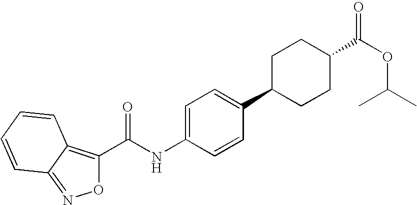 | trans-4-{4- [(Benzo(c)isoxazole- 3-carbonyl)amino]- phenyl}cyclohexane- carboxylic acid isopropyl ester | See Inter- mediate 15 | 4.34 | 407.05 |
| | | | General | | |
| | | | Synthetic | | |
| Ex | Structure | Name | Method | MH⁺ | RT |
| 127 | 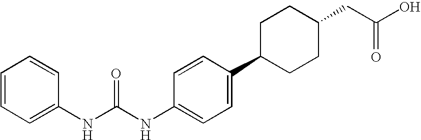 | Trans-{4-[4-(3- Phenylureido)- phenyl]cyclohexyl}- acetic acid | B | 353.05 | 3.36 |
| 128 | 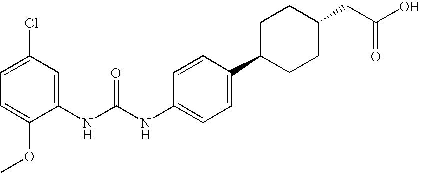 | Trans-(4-{4-[3-(5- Chloro-2-methoxy- phenyl)ureido]- phenyl}cyclohexyl)- acetic acid | B | 417.01 | 3.34 |
| 129 | 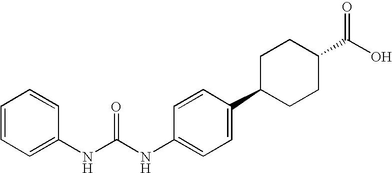 | Trans-4-[4-(3- Phenylureido)- phenyl]cyclohexane carboxylic acid | B | 339.04 | 3.32 |
| 130 | 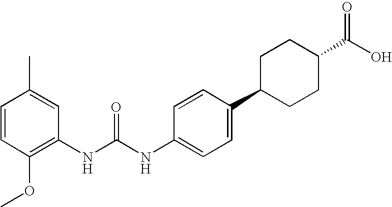 | Trans-4-{4-[3-(2- Methoxy-5-methyl- phenyl)ureido]- phenyl}- cyclohexane- carboxylic acid | See Inter- mediate 27 | 383.03 | 3.51 |
| 131 | 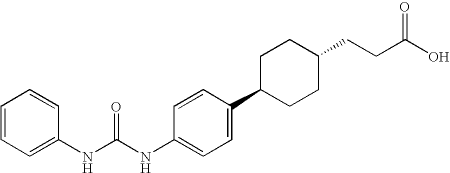 | Trans-3-{4-[4-(3- Phenylureido)- phenyl]- cyclohexyl}propionic acid | B | 367.05 | 3.59 |
| 132 | 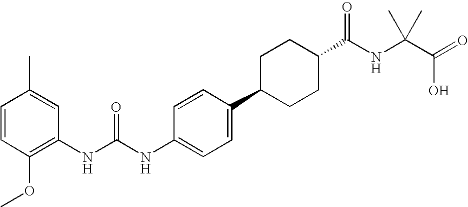 | Trans-2-[(4-{4-[3-(2- Methoxy-5-methyl- phenyl)ureido]- phenyl}- cyclohexane- carbonyl)amino]-2- methylpropionic acid | E | 468.11 | 3.47 |
| 133 | 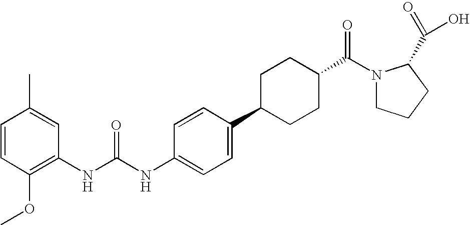 | Trans-(S)-1-(4-{4-[3- (2-Methoxy-5- methyl-phenyl)- ureido]phenyl}- cyclohexane- carbonyl)pyrrolidine- 2-carboxylic acid | E | 480.11 | 3.44 |
| 134 | 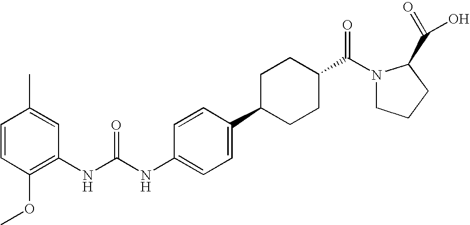 | Trans-(R)-1-(4-{4-[3- (2-Methoxy-5- methylphenyl)- ureido]phenyl}- cyclohexane carbonyl)pyrrolidine- 2-carboxylic acid | E | 480.11 | 3.39 |
| 135 | 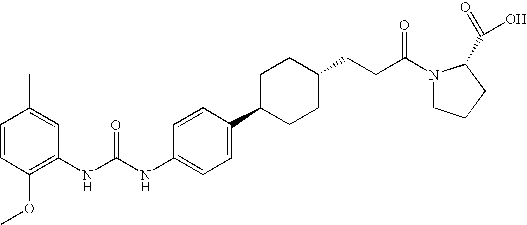 | Trans-(S)-1-[3-(4-{4- [3-(2-Methoxy-5- methylphenyl)- ureido]phenyl}- cyclohexyl)- propionyl]pyrrolidine- 2-carboxylic acid | E | 508.13 | 3.69 |
| 136 | 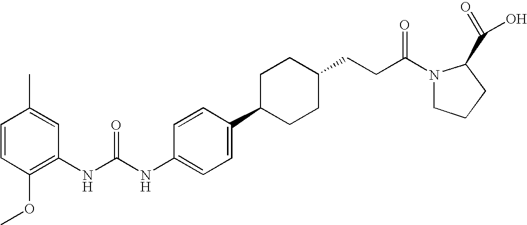 | Trans-(R)-1-[3-(4-{4- [3-(2-Methoxy-5- methylphenyl)- ureido]phenyl}- cyclohexyl)- propionyl]pyrrolidine- 2-carboxylic acid | E | 508.14 | 3.59 |
| 137 | 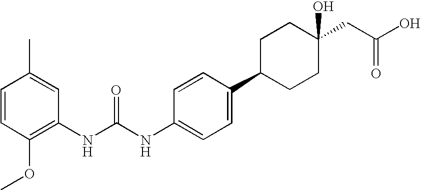 | Syn-(1-Hydroxy-4- {4-[3-(2-methoxy-5- methylphenyl)- ureido]phenyl}- cyclohexyl)-acetic acid | B | 413.08 | 3.36 |
| 138 | 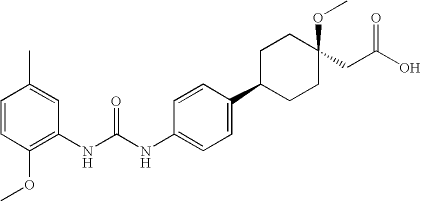 | Syn-(1-Methoxy-4- {4-[3-(2-methoxy-5- methylphenyl)ureido]- phenyl}cyclohexyl)- acetic acid | B | 427.09 | 3.31 |
| 139 | 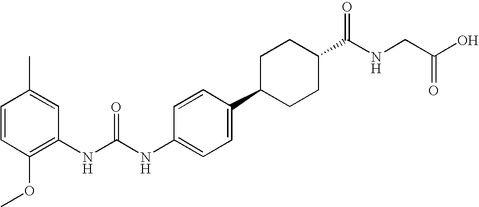 | Trans-[(4-{4-[3-(2- Methoxy-5-methyl- phenyl)ureido]- phenyl}- cyclohexane- carbonyl)amino]- acetic acid | E | 440.12 | 3.31 |
| 140 | 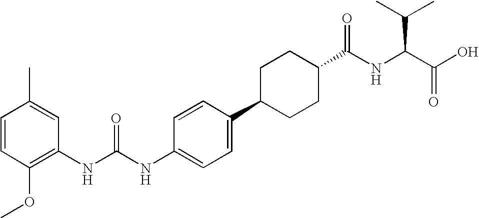 | Trans-(S)-2-[(4-{4- [3-(2-Methoxy-5- methylphenyl)- ureido]phenyl}- cyclohexane- carbonyl)amino]-3- methylbutyric acid | E | 482.15 | 3.61 |
| 141 | 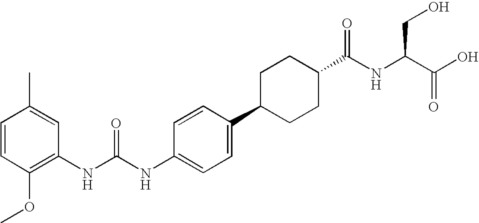 | Trans-(S)-3- Hydroxy-2-[(4-{4-[3- (2-methoxy-5- methylphenyl)- ureido]phenyl}- cyclohexane- carbonyl)amino]- propionic acid | E | 470.12 | 3.20 |
| 142 | 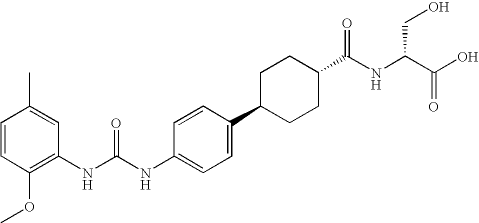 | Trans-(R)-3- Hydroxy-2-[(4-{4-[3- (2-methoxy-5- methylphenyl)- ureido]phenyl}- cyclohexane- carbonyl)amino]- propionic acid | E | 470.12 | 3.20 |
| 143 | 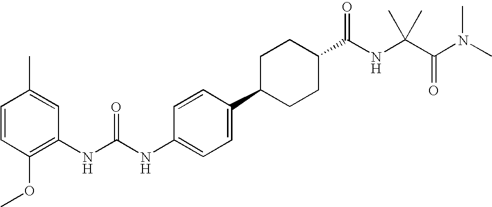 | Trans-4-{4-[3-(2- Methoxy-5-methyl- phenyl)ureido]- phenyl}cyclohexane- carboxylic acid (1- dimethylcarbamoyl- 1-methylethyl)amide | C | 495.20 | 3.42 |
| 144 | 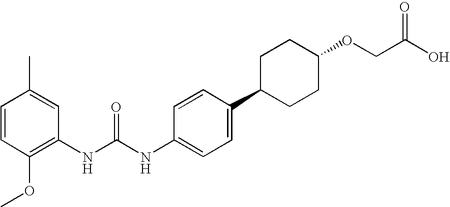 | Trans-4-{4-[3-(2- Methoxy-5-methyl- phenyl)ureido]- phenyl}- cyclohexyloxy)acetic acid | B | 413.09 | 3.61 |
| 145 | 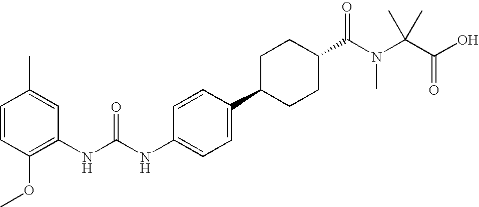 | Trans-2-[(4-{4-[3-(2- Methoxy-5-methyl- phenyl)ureido]- phenyl}cyclohexane- carbonyl)methyl- amino]-2-methyl- propionic acid | B | 482.15 | 3.51 |
| 146 | 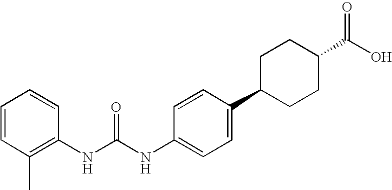 | Trans-4-[4-(3-o- Tolyl-ureido)phenyl]- cyclohexane- carboxylic acid | B | 353.12 | 3.42 |
| 147 | 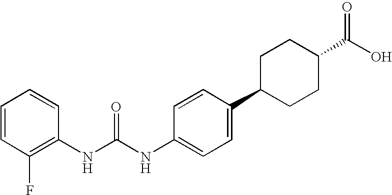 | Trans-4-{4-[3-(2- Fluorophenyl)- ureido]phenyl}- cyclohexane- carboxylic acid | B | 357.07 | 3.52 |
| 148 | 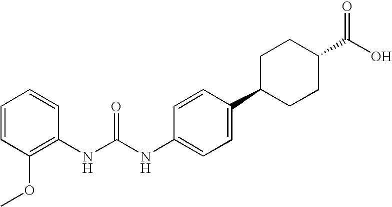 | Trans-4-{4-[3-(2- Methoxyphenyl)- ureido]phenyl}- cyclohexane- carboxylic acid | B | 369.05 | 3.45 |
| 149 | 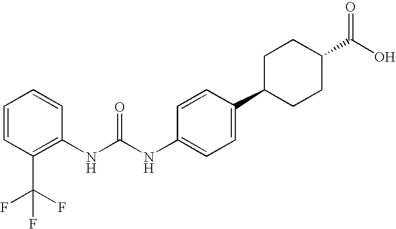 | Trans-4-{4-[3-(2- Trifluoromethyl- phenyl)ureido]- phenyl}cyclohexane- carboxylic acid | B | 407.03 | 3.59 |
| 150 | 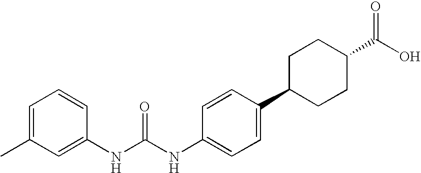 | Trans-4-[4-(3-m- Tolylureido)phenyl]- cyclohexane- carboxylic acid | B | 353.07 | 3.46 |
| 151 | 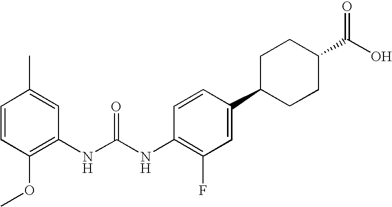 | Trans-4-{3-Fluoro-4- [3-(2-methoxy-5- methylphenyl)- ureido]phenyl}- cyclohexane- carboxylic acid | B | 401.43 | 3.67 |
| 152 | 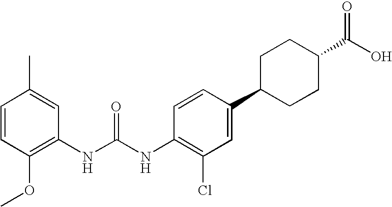 | Trans-4-{3-Chloro-4- [3-(2-methoxy-5- methylphenyl)- ureido]pehnyl}- cyclohexane- carboxylic acid | B | 417.04 | 3.76 |
| 153 | 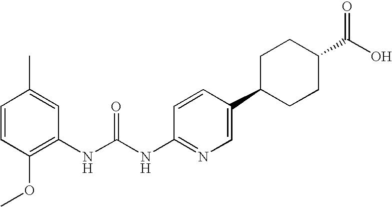 | Trans-4-{6-[3-(2- Methoxy-5-methyl- phenyl)ureido]- pyridin-3-yl}- cyclohexane- carboxylic acid | B | 383.97 | 3.92 |
| 154 | 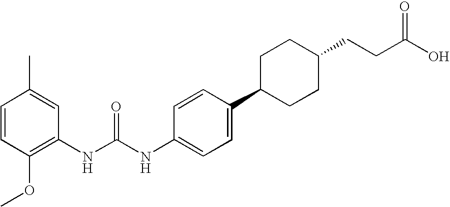 | Trans-3-(4-{4-[3-(2- Methoxy-5- methylphenyl)ureido]- phenyl}cyclohexyl)- propionic acid | B | 411.07 | 3.70 |
| 155 | 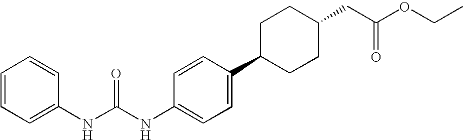 | Trans-{4-[4-(3- Phenylureido)- phenyl]cyclohexyl}- acetic acid ethyl ester | A | 381.04 | 3.57 |
| 156 | 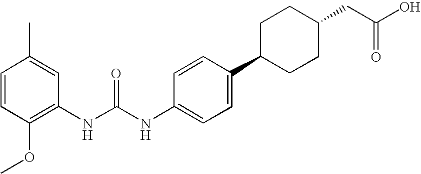 | Trans-(4-{4-[3-(2- Methoxy-5-methyl- phenyl)ureido]- phenyl}cyclohexyl) acetic acid | B | 397.06 | 3.74 |
| 157 | 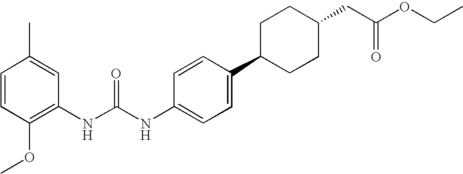 | Trans-(4-{4-[3-(2- Methoxy-5-methyl- phenyl)ureido]- phenyl}cyclohexyl) acetic acid ethyl ester | A | 425.06 | 3.90 |
| 158 | 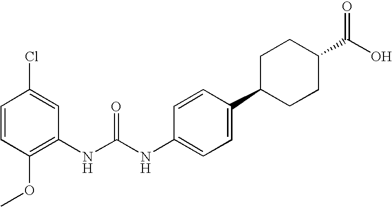 | Trans-4-{4-[3-(5- Chloromethoxy- phenyl)ureido]- phenyl}cyclohexane- carboxylic acid | B | 417.01 | 3.34 |
| 159 | 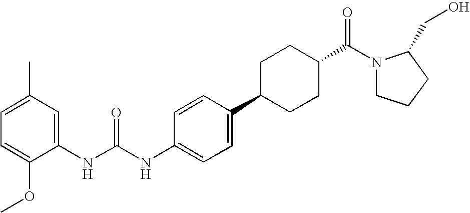 | Trans-1-{4-[4-((S)-2- Hydroxymethyl- pyrrolidine-1- carbonyl)- cyclohexyl]phenyl}- 3-(2-methoxy-5- methylphenyl)urea | C | 466.13 | 3.56 |
| 160 | 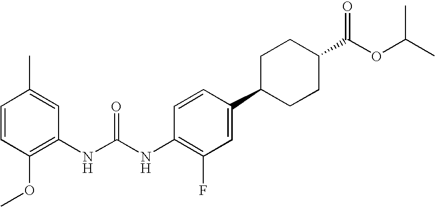 | Trans-4-{3-Fluoro-4- [3-(2-methoxy-5- methylphenyl)- ureido]phenyl}- cyclohexane- carboxylic acid isopropyl ester | A | 443.09 | 4.42 |
| 161 | 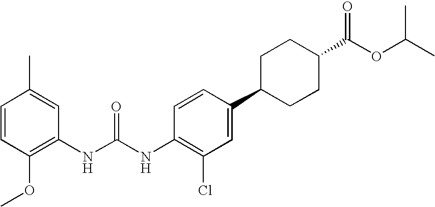 | Trans-4-{3-Chloro-4- [3-(2-methoxy-5- methylphenyl)- ureido]phenyl}- cyclohexane- carboxylic acid isopropyl ester | A | 459.08 | 4.45 |
| 162 | 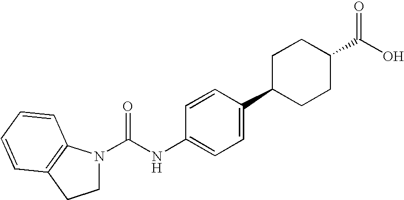 | Trans-4-{4-[(2,3- Dihydroindole-1- carbonyl)amino]- phenyl}cyclohexane- carboxylic acid | D | 365.23 | 3.50 |
| 163 | 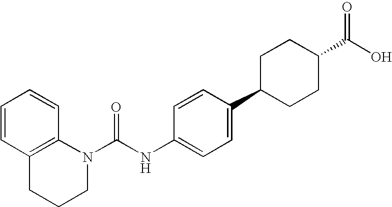 | Trans-4-{4-[(3,4- Dihydro-2H- quinoline-1- carbonyl)amino]- phenyl}cyclohexane- carboxylic acid | D | 379.15 | 3.49 |
| 164 | 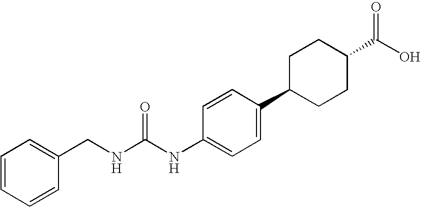 | Trans-4-[4-(3- Benzylureido)- phenyl]cyclohexane- carboxylic acid | D | 353.09 | 3.14 |
| 165 | 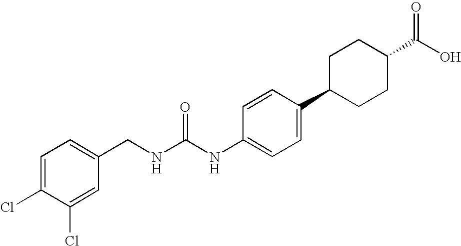 | Trans-4-{4-[3-(3,4- Dichlorobenzyl)- ureido]phenyl}- cyclohexane- carboxylic acid | D | 420.99 | 3.59 |
| 166 | 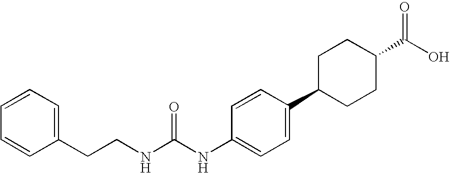 | Trans-4-[4-(3- Phenethylureido)- phenyl]cyclohexane- carboxylic acid | D | 368.07 | 3.27 |
| 167 | 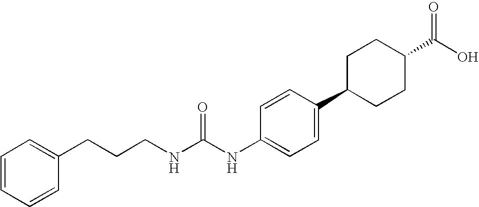 | Trans-4-{4-[3-(3- Phenpropyl)ureido]- phenyl}cyclohexane- carboxylic acid | D | 382.11 | 3.39 |
| 168 | 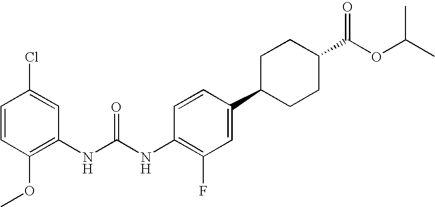 | Trans-4-{4-[3-(5- Chloro-2-methoxy- phenyl)ureido]-3- fluorophenyl}- cyclohexane- carboxylic acid isopropyl ester | A | 463.03 | 4.49 |
| 169 | 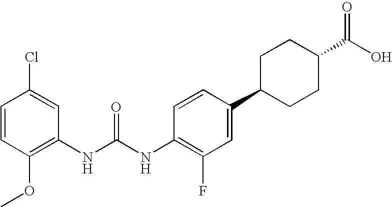 | Trans-4-{4-[3-(5- Chloro-2-methoxy- phenyl)ureido]-3- fluorophenyl}- cyclohexane- carboxylic acid | B | 420.97 | 3.87 |
| 170 | 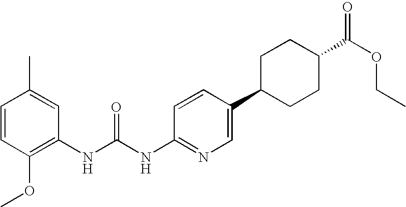 | Trans-4-{6-[3-(2- Methoxy-5-methyl- phenyl)ureido]- pyridin-3-yl}- cyclohexane- carboxylic acid ethyl ester | See Inter- mediate 27 | 412.00 | 3.92 |
| 171 | 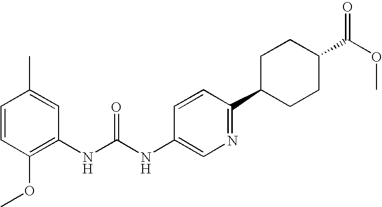 | Trans-4-{5-[3-(2- Methoxy-5-methyl- phenyl)ureido]- pyridin-3-yl}- cyclohexane- carboxylic acid methyl ester | A | 397.98 | 2.86 |
| 172 | 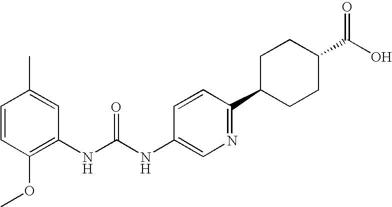 | Trans-4-{5-[3-(2- Methoxy-5-methyl- phenyl)ureido]- pyridin-3-yl}- cyclohexane- carboxylic acid | B | 383.93 | 2.72 |
| 173 | 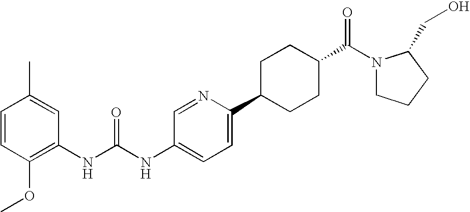 | Trans-1-{6-[4-((S)-2- Hydroxymethyl- pyrrolidine-1- carbonyl)- cyclohexyl]pyridin-3- yl}-3-(2-methoxy-5- methylphenyl)urea | C | 466.98 | 2.64 |
| 174 | 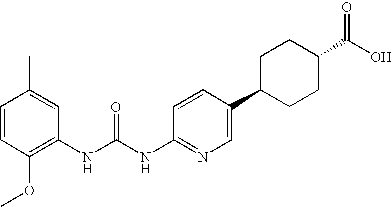 | Trans-4-{6-[3-(2- Methoxy-5-methyl- phenyl)ureido]- pyridin-3-yl}- cyclohexane- carboxylic acid | B | 383.92 | 3.22 |
| 175 | 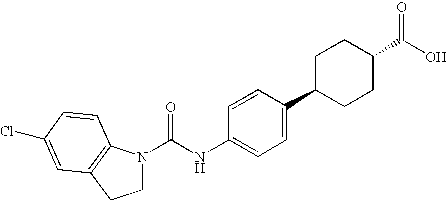 | Trans-4-{4-[(5- Chloro-2,3- dihydroindole-1- carbonyl)amino]- phenyl}cyclohexane- carboxylic acid | B | 399.04 | 3.67 |
| 176 | 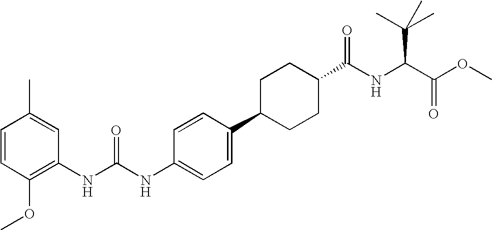 | Trans-(S)-2-[(4-{4- [3-(2-Methoxy-5- methylphenyl)- ureido]phenyl}- cyclohexane carbonyl)amino]-3,3- dimethylbutyric acid methyl ester | A | 510.0 | 4.12 |
| 177 | 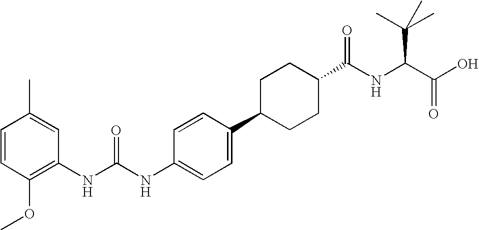 | Trans-(S)-2-[(4-{4- [3-(2-Methoxy-5- methylphenyl)- ureido]phenyl}- cyclohexane carbonyl)amino]-3,3- dimethylbutyric acid | E | 496.01 | 3.76 |
| 178 | 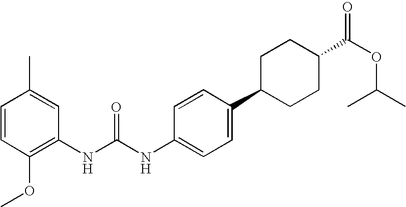 | Trans-4-{4-[3-(2- Methoxy-5- methylphenyl)ureido] phenyl}cyclohexane carboxylic acid isopropyl ester | See Inter- mediate 27 | 425.14 | 4.26 |
| 179 | 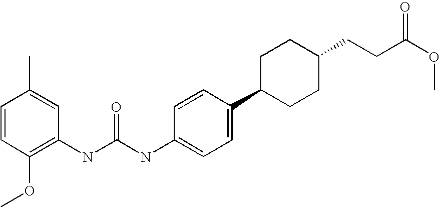 | Trans-3-(4-{4-[3-(2- Methoxy-5- methylphenyl)ureido]- phenyl}cyclohexyl)- propionic acid methyl ester | See Inter- mediate 27 | 425.09 | 4.02 |
| 180 | 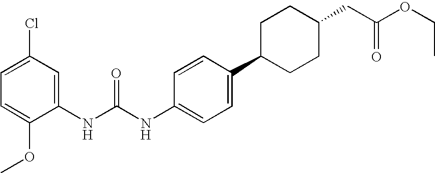 | Trans-(4-{4-[3-(5- Chloro-2-methoxy- phenyl)ureido]- phenyl}cyclohexyl)- acetic acid ethyl ester | See Inter- mediate 27 | 445.01 | 4.34 |
| 181 | 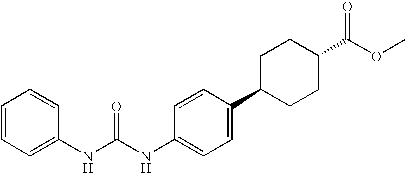 | Trans-4-[4-(3- Phenylureido)- phenyl]cyclohexane carboxylic acid methyl ester | See Inter- mediate 27 | 353.06 | 3.67 |
| 182 | 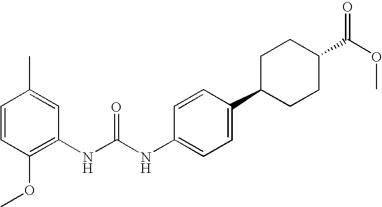 | Trans-4-{4-[3-(2- Methoxy-5-methyl- phenyl)ureido]- phenyl}- cyclohexane- carboxylic acid methyl ester | See Inter- mediate 27 | 397.06 | 3.89 |
| 183 | 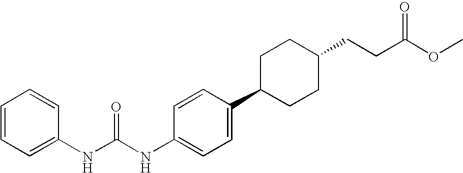 | Trans-3-{4-[4-(3- Phenylureido)- phenyl]- cyclohexyl}propionic acid methyl ester | See Inter- mediate 27 | 381.08 | 3.97 |
| 184 | 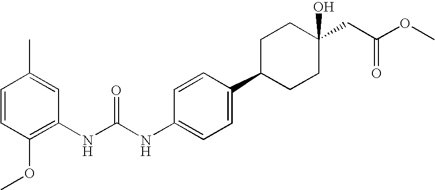 | Syn-(1-Hydroxy-4- {4-[3-(2-methoxy-5- methylphenyl)- ureido]phenyl}- cyclohexyl)-acetic acid methyl ester | See Inter- mediate 27 | 427.11 | 3.49 |
| 185 | 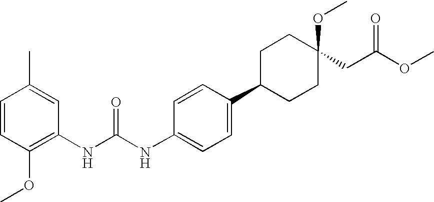 | Syn-(1-Methoxy-4- {4-[3-(2-methoxy-5- methylphenyl)ureido]- phenyl}cyclohexyl)- acetic acid methyl ester | See Inter- mediate 27 | 441.12 | 3.90 |
| 186 | 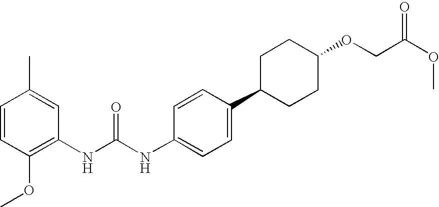 | Trans-(4-{4-[3-(2- Methoxy-5-methyl- phenyl)ureido]- phenyl}- cyclohexyloxy)acetic acid methyl ester | See Inter- mediate 27 | 427.13 | 3.84 |
| 187 | 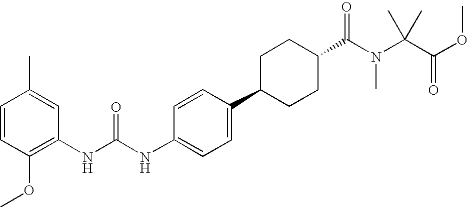 | Trans-2-[(4-{4-[3-(2- Methoxy-5-methyl- phenyl)ureido]- phenyl}cyclohexane- carbonyl)methyl- amino]-2-methyl- propionic acid methyl ester | See Inter- mediate 27 | 496.11 | 3.84 |
| 188 | 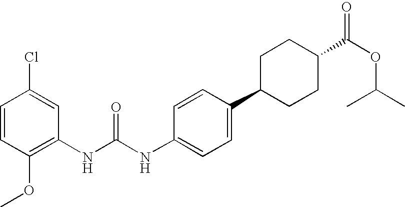 | Trans-4-{4-[3-(5- Chloromethoxy- phenyl)ureido]- phenyl}cyclohexane- carboxylic acid isopropyl ester | See Inter- mediate 27 | 445.07 | 4.54 |
| 189 | 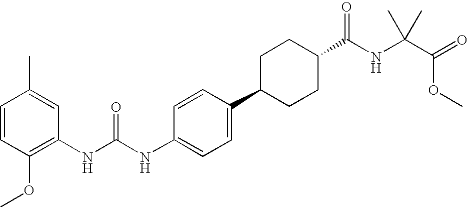 | trans-2-[(4-{4-[3-(2- Methoxy-5- methylphenyl)ureido] phenyl}cyclohexane- carbonyl)-amino]-2- methylpropionic acid methyl ester | See Inter- mediate 44 | 484.10 | 3.64 |
| 190 | 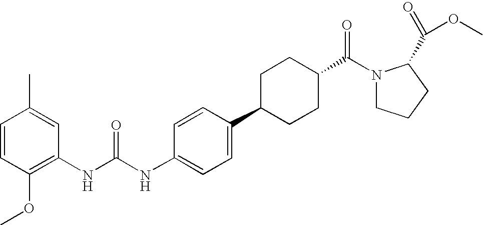 | trans-(S)-1-(4-{4-[3- (2-Methoxy-5- methylphenyl)- ureido]phenyl}- cyclohexane- carbonyl)pyrrolidine- 2-carboxylic acid methyl ester | See Inter- mediate 44 | 494.11 | 3.76 |
| 191 | 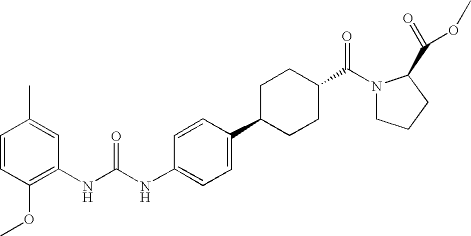 | trans-(R)-1-(4-{4-[3- (2-Methoxy-5- methylphenyl)- ureido]phenyl}- cyclohexane- carbonyl)pyrrolidine- 2-carboxylic acid methyl ester | See Inter- mediate 44 | 494.11 | 3.65 |
| 192 | 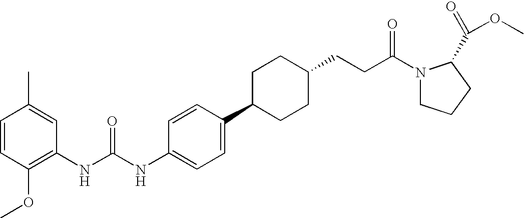 | trans-(S)-1-[3-(4-{4- [3-(2-Methoxy-5- methylphenyl)ureido] methylphenyl)ureido] phenyl}cyclohexyl)- propionyl]pyrrolidine- 2-carboxylic acid methyl ester. | See Inter- mediate 44 | 522.13 | 3.99 |
| 193 | 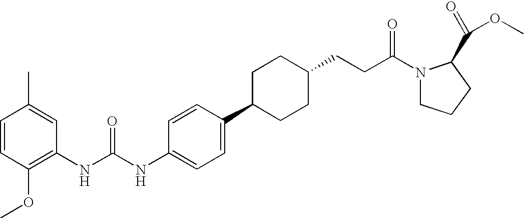 | trans-(R)-1-[3-(4-{4- [3-(2-Methoxy-5- methylphenyl)ureido] phenyl}cyclohexyl)- propionyl]pyrrolidine- 2-carboxylic acid methyl ester | See Inter- mediate 44 | 522.11 | 3.90 |
| 194 | 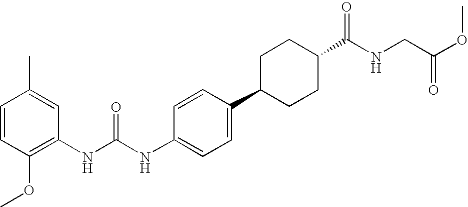 | trans-[(4-{4-[3-(2- Methoxy-5-methyl- phenyl)ureido]- phenyl}- cyclohexane- carbonyl)amino]- acetic acid methyl ester | See Inter- mediate 44 | 454.15 | 3.56 |
| 195 | 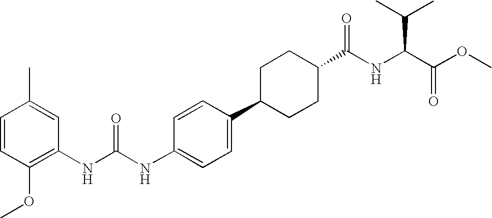 | trans-(S)-2-[(4-{4-[3- (2-Methoxy-5- methylphenyl)ureido] phenyl}cyclohexane- carbonyl)amino]-3- methylbutyric acid methyl ester. | See Inter- mediate 44 | 496.17 | 3.79 |
| 196 | 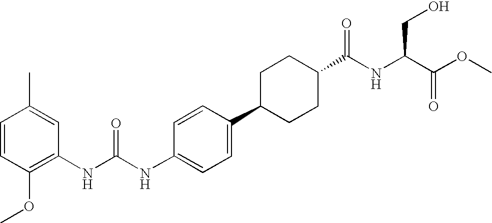 | trans-(S)-3-Hydroxy- 2-[(4-{4-[3-(2- methoxy-5- methylphenyl)ureido] phenyl}- cyclohexane- carbonyl)amino]- propionic acid methyl ester | See Inter- mediate 44 | 484.16 | 3.37 |
| 197 | 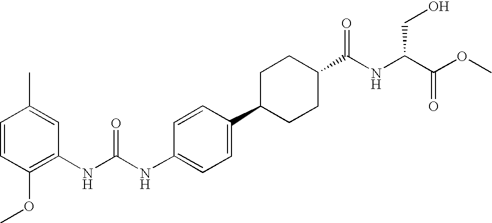 | trans-(R)-3-Hydroxy- 2-[(4-{4-[3-(2- methoxy-5- methylphenyl)ureido] phenyl}- cyclohexane- carbonyl)amino]- propionic acid methyl ester | See Inter- mediate 44 | 484.15 | 3.32 |

The biological activity of the compounds of the invention may be tested in the following assay systems:

### Evaluation of DGAT1 Enzyme Inhibition

DGAT assays for compound screening were carried out in 96-well round bottom plates in a total volume of 50 μl containing 25 mM Hepes pH 7.4, 150 mM NaCl, 2 mM MgCl₂, 20 mM sodium fluoride, 1 mM dithiothreitol, 0.5 mg/mL BSA, 0.1% Triton X100, 200 uM DAG (1,2-dioleoyl-sn-glycerol) and 15 μM [¹⁴C] oleoyl CoA (specific activity 50-60 mCi/mmol) and a range of inhibitor concentrations in 1% DMSO. Reactions were initiated by the addition of human DGAT1 microsomes (0.4 μg/well) followed by incubation at 30° C. for 20 min. At the end of incubation, reactions were stopped by the addition of 5 μl 0.5N HCl, then 4 μl of each reaction was spotted directly onto a silica gel 60 (20×20 cm) TLC plate pre-scored to give twenty 1 cm wide lanes for lipid separation. Each lane was also spotted with 2 μl of carrier triglyceride (5 mg/mL) made up in chloroform/methanol (2:1 v/v mixture). After drying, the TLC plate was resolved over a distance of 15 cm in a solvent consisting of hexane, diethyl ether and acetic acid at a ratio of 80:20:1 v/v. The triglyceride spots were then located by iodine vapour staining.

Following chromatography, the triglyceride spots were cut out and placed in a scintillation vial for counting. The percentage inhibition values were calculated relative to control and where appropriate IC₅₀ values were derived using the 4 parameter logistic model in XL fit. Generally compounds of the invention showed IC₅₀ values below 5 μM, thus, for example, the compound of Example 114 showed an IC₅₀ value of 1.8 μM and the compound of Example 137 showed an IC₅₀ value of 1.3 μM.

The enzyme source used for the assay was prepared from Sf21 cells infected with baculovirus expressing human DGAT1. Full length human DGAT10RF (1467 bp) (Nucleotide Accession: NM 012079; Protein Accession: NP036211) was PCR amplified from human liver cDNA (Marathon Ready) and cloned into pFastBac HTa vector (Life Technologies) containing a 6-Histidine N-terminus. Subsequent baculovirus generation was carried out using Invitrogen's Bac to Bac system to yield optimal protein expression in Sf21 insect cells after 48 h of infection at a multiplicity of infection of 5 pfu/cell.

### Preparation of Microsomes

Sf21 cells infected with human DGAT1 recombinant baculovirus were washed in ice-cold phosphate buffered saline and resuspended in a buffer containing 20 mM Hepes pH 7.5, 1 mM CaCl₂, 1 mM MgCl₂, 1 mM dithiothreitol and Complete Protease Inhibitor cocktail (Roche) 48 hours post infection. The suspended cells were allowed to swell on ice for 10 min after which time they were lysed with 15 strokes of a Dounce homogeniser (pestle B). Sucrose was then added to the lysate to a final concentration of 7.5% and the mixture was centrifuged at 1,500 g for 10 min at 4° C. The supernatant was collected and further centrifuged at 110,000 g for 1 h at 4° C. The resulting pellet, constituting the microsome fraction, was finally resuspended in 20 mM Hepes pH 7.5, 0.25M sucrose and Complete Protease Inhibitor and stored at −80° C. The protein concentration was determined using the Bio-Rad protein assay reagent kit.

### Evaluation of Inhibition of Cellular Triglyceride Synthesis

The cell-based assay for DGAT is conducted with mouse 3T3L1 adipocytes. The 3T3L1 cells are grown in 24 well plates in DMEM supplemented with 10% FBS, Penicillin and Streptomycin. Triglyceride formation is determined in day 0 adipocytes by the addition of 0.25 μCi of [1 ¹⁴C]-oleic acid (55 mCi/mmol) in the presence of a range of concentrations of inhibitors using DMSO as the carrier with a maximum DMSO concentration of 0.25%. After 16 h the cells are washed with PBS followed by lysis in 1 mM EDTA. The lysates are transferred to glass tubes and the lipids extracted by the addition of 2:1 (v/v) of chloroform:methanol and 1M H₂SO₄. The organic phase is removed, dried down and reconstituted in hexane. 4 μl of the reconstituted sample is then applied to silica coated TLC plates along with non-labelled triolein as carrier and standard. The TLC plates are developed in solvent system of n-hexane:diethyl ether:acetic acid (80:20:1). The plates are then exposed to iodine vapour, triglycerides spots visualised and marked before exposure of the plates to X-ray film. Following autoradiography, the triglyceride spots are excised from the TLC and counted by liquid scintillation counting. EC₅₀ values are then determined as the inhibitor concentration at 50% of the dynamic range of the ¹⁴C dpm incorporation. Generally compounds showed EC₅₀ values below 10 μM.

What is claimed is:

1. A compound of formula (I) 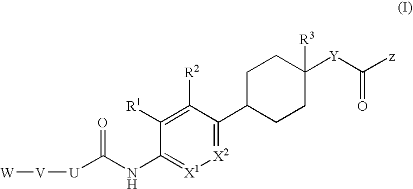
one of X¹ and X² is N and the other is CR⁴;
R¹, R² and R⁴ independently represent a group selected from hydrogen, F, Cl, methyl and methoxy;
R³ represents hydrogen, C₁-C₃ alkyl, hydroxy or C₁-C₃ alkoxy;
Y represents a bond, or —(CH₂)ₙ(CR¹⁰R¹³)ₒ(CH₂)ₚ—; or when R³ represents hydrogen or C₁-C₃ alkyl then Y may also represent —O—(CH₂)ₙ(CR¹⁰R¹³)ₒ(CH₂)ₚ—;
n represents an integer 0 to 3;
o represents 0 or 1;
p represents an integer 0 to 3;
provided that n+o+p is 1, 2 or 3;
Z represents hydroxy, C₁-C₆ alkoxy or —NR⁶R⁷;
R⁶ represents hydrogen, C₁-C₆ alkyl or C₃-C₆ cycloalkyl either of which groups may optionally be substituted by one or two groups selected from hydroxy, C₁-C₃ alkoxy, —NR⁸R⁹, phenyl, —CONR¹¹R¹² and COOR^{S}, provided that there are at least two carbon atoms between the nitrogen of the —NR⁶R⁷ group and any hydroxy, —C₁-C₃ alkoxy, or —NR⁸R⁹ substituent on R⁶;
R⁷ represents hydrogen or C₁-C₆ alkyl;
or R⁶ and R⁷ are joined such that —NR⁶R⁷ forms a 5- to 7-membered heterocyclic ring optionally containing an additional heteroatom selected from N, O and S, which ring may optionally be substituted by a group selected from hydroxy, C₁-C₃ alkoxy, C₁-C₃ alkyl, CH₂OH, CH₂OMe and COOH;
U represents a bond or >N-Q;
Q represents hydrogen, or Q is joined to W such that WVNQ together form a 5- to 7-membered nitrogen containing heterocyclic ring fused to a phenyl which may optionally be substituted as for W;
when U represents a bond, V represents a bond, (CH₂)ₘ which may be optionally substituted by C₁-C₃ alkyl, hydroxy or C₁-C₃ alkoxy, —(CH₂)ₐO(CH₂)_{b}—, —(CH₂)_{c}OCH(Me)—, —NR¹⁴(CH₂)_{d}— or cyclopropyl in which the points of attachment are 1,1 or 1,2;
when U represents >N-Q, V represents a bond or (CH₂)ₘ;
m represents an integer 1 to 3;
a represents 0 or 1;
b represents 1 or 2;
c represents 0 or 1;
d represents 1 or 2;
R⁵, R⁸, R⁹, R¹¹, R¹² and R¹⁴ independently represent hydrogen or C₁-C₃ alkyl;
R¹⁰ and R¹³ independently represent hydrogen or C₁-C₃ alkyl or, together with the carbon atom to which they are attached, can be joined to form a C₃-C₆ cycloalkyl ring; and
W represents a 5- to 10-membered monocyclic or bicyclic aromatic or heteroaromatic ring, which bicyclic rings may contain one unsaturated ring; any of said rings being optionally substituted by one or more groups selected from halo, hydroxy, cyano, C₁-C₆ alkyl or C₁-C₃ alkoxy, either of which may be substituted by one or more fluoro atoms; and when W is monocyclic may also be optionally substituted by phenyl, a 5- to 6-membered heteroaromatic, 5- to 7-membered heterocyclic or C₃-C₆ cycloalkyl ring;
or a pharmaceutically acceptable salt or ester thereof.

2. A compound of formula (I) according to claim 1, or a pharmaceutically acceptable salt or ester thereof, wherein U represents a bond.

3. A compound of formula (I) according to claim 1, or a pharmaceutically acceptable salt or ester thereof, wherein U represents >N-Q.

4. A compound of formula (I) according to claim 1, or a pharmaceutically acceptable salt or ester thereof, wherein R³ represents hydrogen.

5. A compound of formula (I) according to claim 1, or a pharmaceutically acceptable salt or ester thereof, wherein X¹ represents CR⁴.

6. A compound of formula (I) according to claim 1, or a pharmaceutically acceptable salt or ester thereof, wherein X² represents CR⁴.

7. A compound of formula (I) according to claim 5, or a pharmaceutically acceptable salt or ester thereof, wherein R⁴ represents hydrogen.

8. A compound of formula (I) according to claim 1, or a pharmaceutically acceptable salt or ester thereof, wherein Q represents hydrogen.

9. A compound of formula (I) according to claim 1, or a pharmaceutically acceptable salt or ester thereof, wherein Y represents a bond.

10. A compound of formula (I) according to claim 1, or a pharmaceutically acceptable salt or ester thereof, wherein Y represents CH₂.

11. A compound of formula (I) according to claim 1, or a pharmaceutically acceptable salt or ester thereof, wherein Z represents hydroxy.

12. A compound of formula (I) according to claim 1, or a pharmaceutically acceptable salt or ester thereof, wherein Z represents C₁-C₆ alkoxy.

13. A compound of formula (I) according to claim 1, or a pharmaceutically acceptable salt or ester thereof, wherein Z represents —NR⁶R⁷.

14. A compound of formula (I) as defined in claim 1, or a pharmaceutically acceptable salt or ester thereof, wherein V represents a bond.

15. A compound of formula (I) as defined in claim 1, or a pharmaceutically acceptable salt or ester thereof, wherein W represents optionally substituted phenyl, naphthyl, benzo[c]isoxazole, pyrazole, quinoline, pyridine, isoxazole or benzofuran.

16. A compound of formula (I) as defined in claim 1, or a pharmaceutically acceptable salt or ester thereof, wherein the stereochemical orientation of the
—YC(O)Z group to the aromatic ring across the cyclohexane ring is trans.

17. (canceled)

18. A pharmaceutical composition comprising a compound according to claim 1, or a pharmaceutically acceptable salt or ester thereof, and a pharmaceutically acceptable carrier.

19. A method for the treatment of a disease or condition in which inhibition of DGAT1 plays a role comprising a step of administering to a subject in need thereof an effective amount of a compound according to claim 1, or a pharmaceutically acceptable salt or ester thereof, such that a disease or condition in which inhibition of DGAT1 plays a role is thereby treated.

20. A method for the treatment of a disease or condition in which inhibition of DGAT1 is desirable comprising a step of administering to a subject in need thereof an effective amount of a compound according to claim 1, or a pharmaceutically acceptable salt or ester thereof, such that a disease or condition in which inhibition of DGAT1 is desirable is thereby treated.

21. A method for the treatment of obesity comprising a step of administering to a subject in need thereof an effective amount of a compound according to claim 1, or a pharmaceutically acceptable salt or ester thereof, such that obesity is thereby treated.

22. A method for the treatment of a metabolic disease selected from Type II diabetes, metabolic syndrome (syndrome X), impaired glucose tolerance, dyslipidemia, hyperlipidemia, hypertriglyceridemia, hypercholesterolemia, low HDL levels and hypertension, comprising a step of administering to a subject in need thereof an effective amount of a compound according to claim 1, or a pharmaceutically acceptable salt or ester thereof, such that said metabolic disease is thereby treated.

23. A process for the preparation of a compound of formula (I) as defined in claim 1, or a pharmaceutically acceptable salt or ester thereof, which comprises:
a) reaction of a compound of formula (II), or a protected derivative thereof, 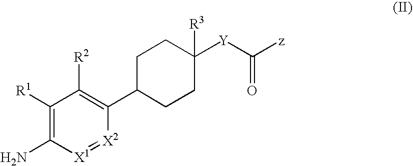
wherein X¹, X², R¹, R², R³, Y and Z are as defined in claim 1;
with, when U is a bond, a compound of formula (III), or a protected or activated derivative thereof:
W—V—COOH (III)
or when U is >N-Q, with a compound of formula (IIIA) or (IIIB), or a protected derivative of either thererof: 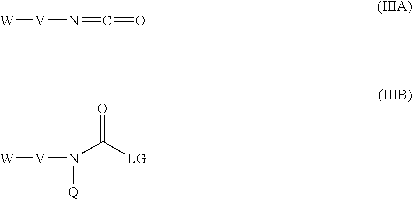
wherein LG is a leaving group, and wherein Q, W and V are as defined in any one of claims 1 to 17; or
b) when U is >N-Q, reaction of a compound of formula (XXVII): 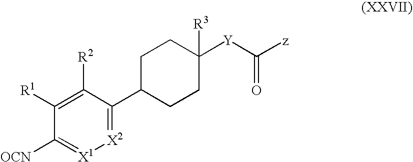
or a compound of formula (XXVIII): 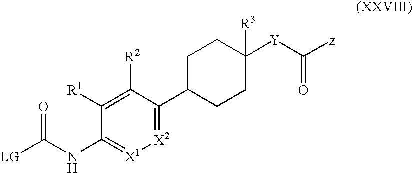
or a protected derivative of either thereof; where LG is a leaving group, and wherein X¹, X², R¹, R², R³, Y and Z are as defined in claim 1, with a compound of formula (XXIX):
W—V—NQH (XXIX)
or a protected derivative thereof, wherein Q, W and V are as defined in claim 1; and
c) if required, interconversion of group —YC(O)Z to a further group —YC(O)Z as defined in claim 1.

24. A compound selected from the following: 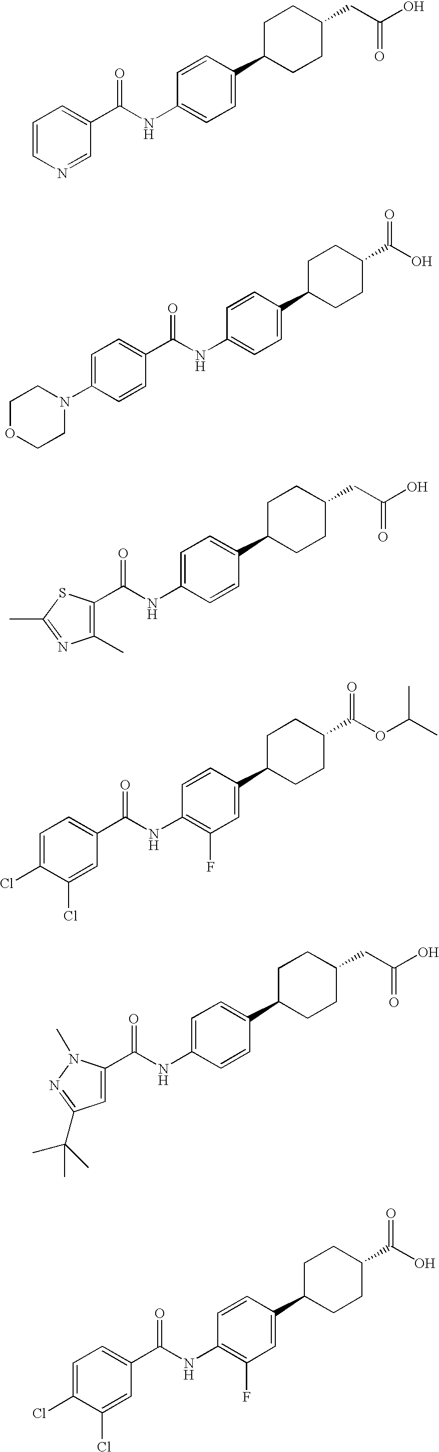 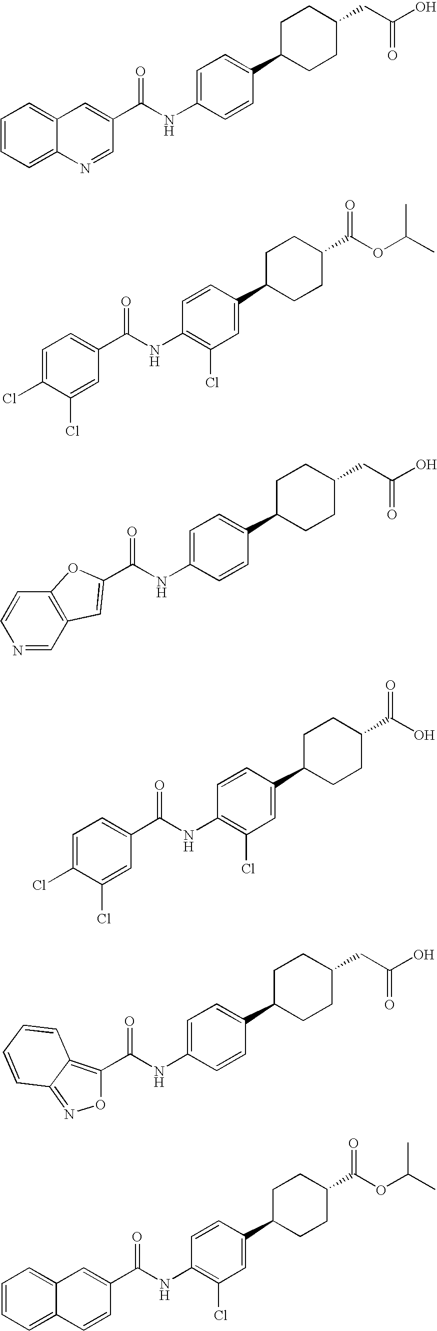 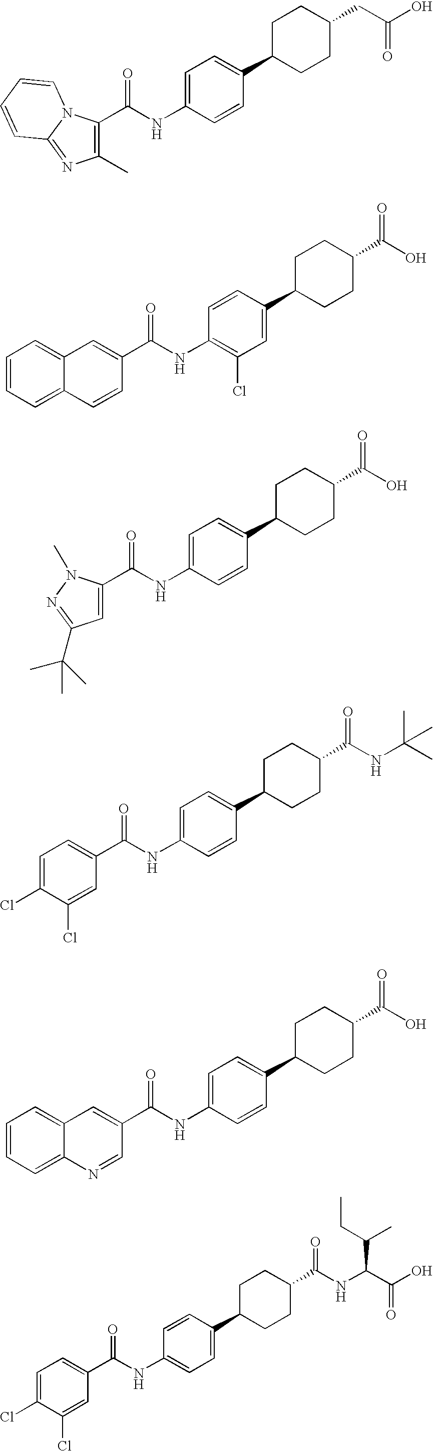 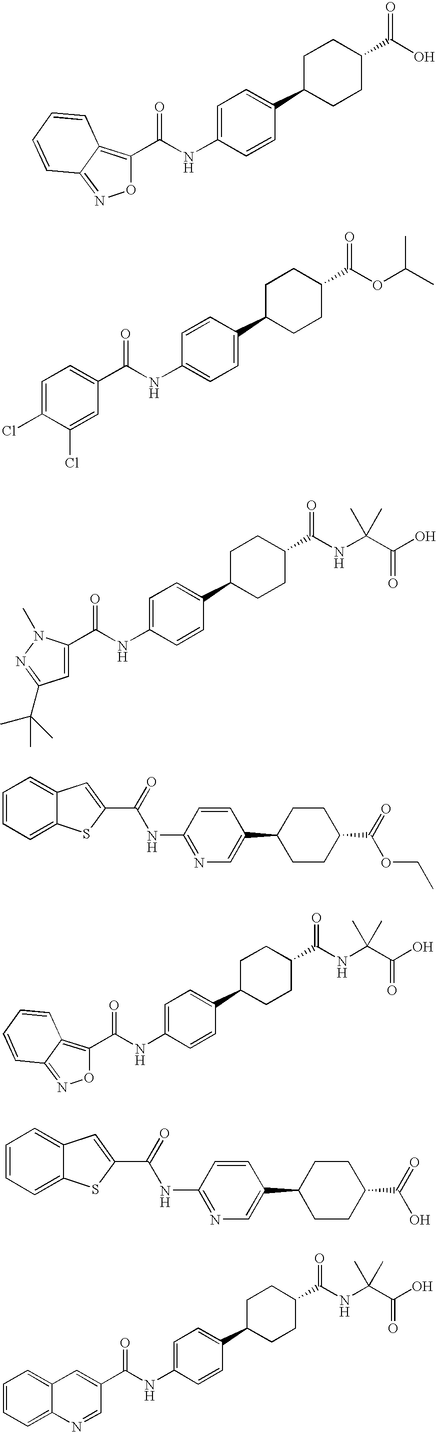 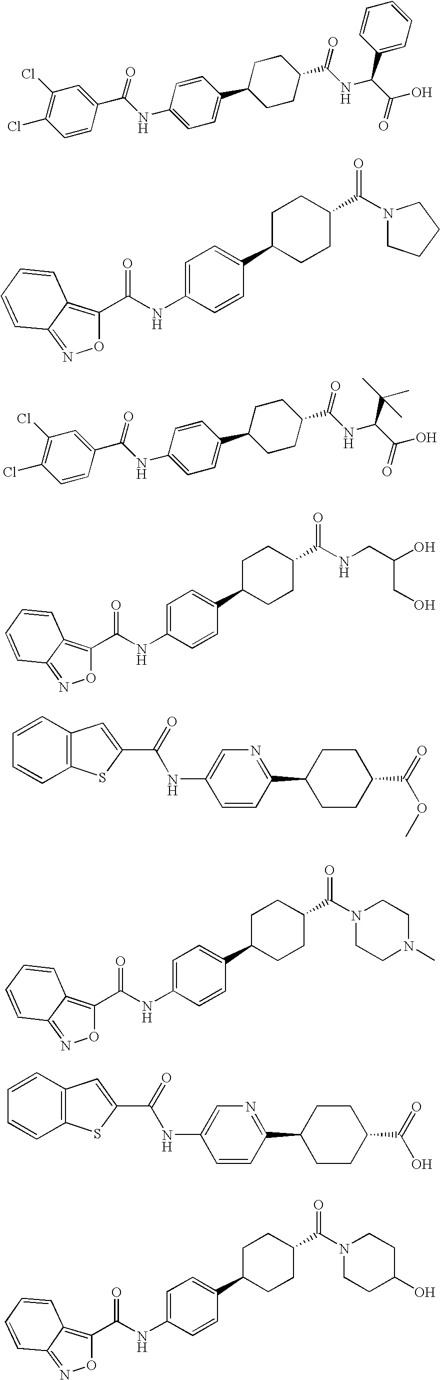 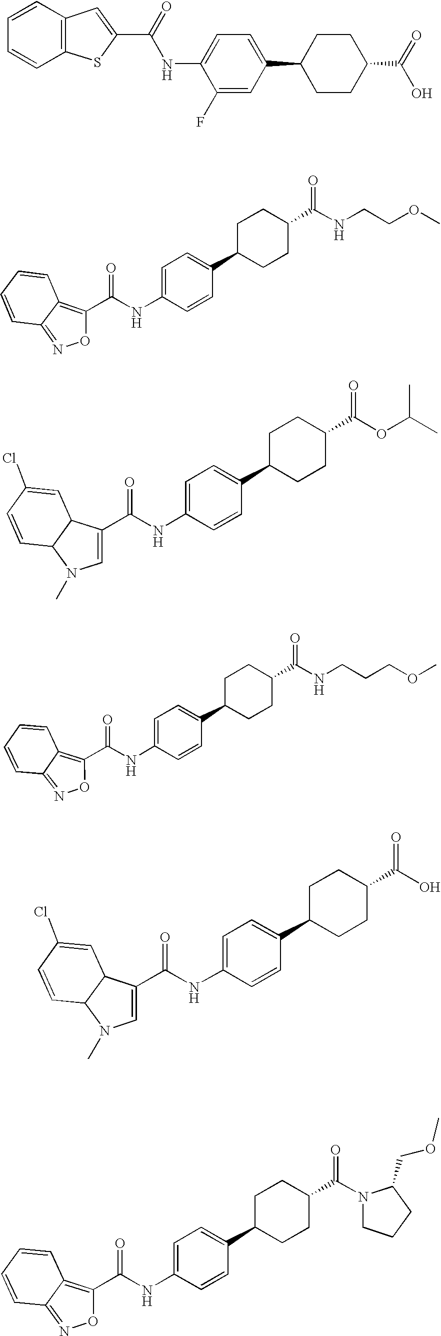 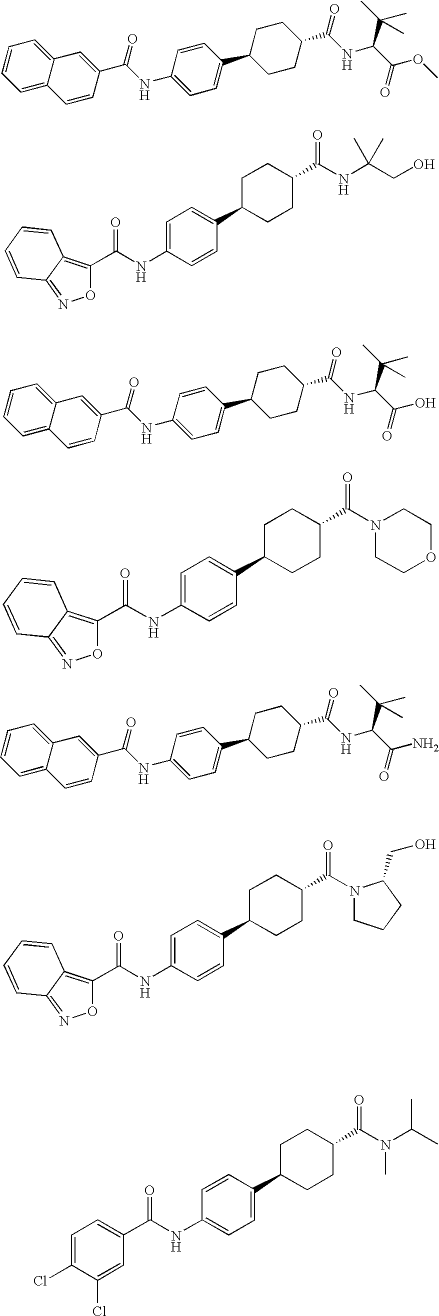 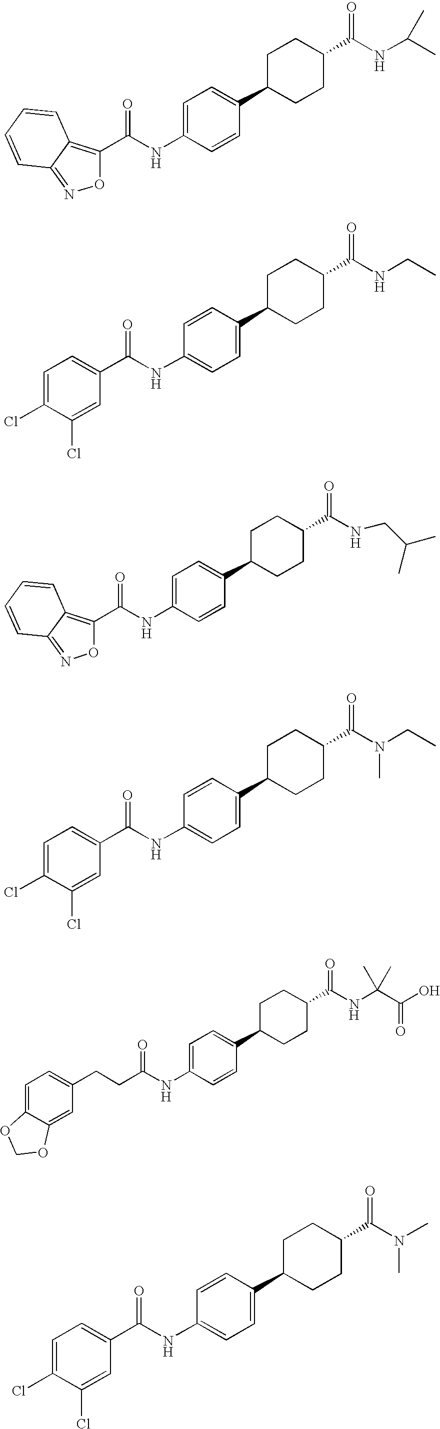 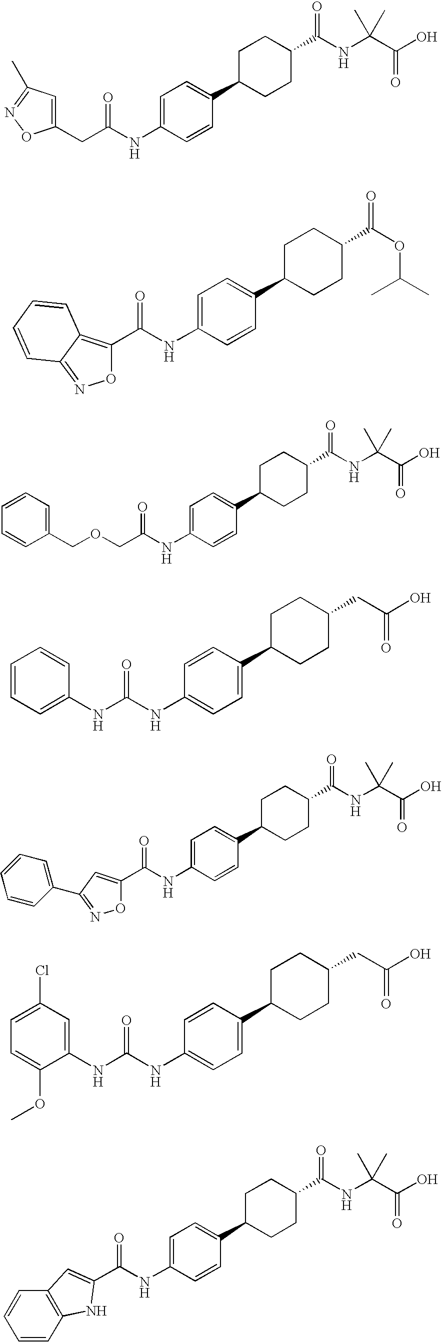 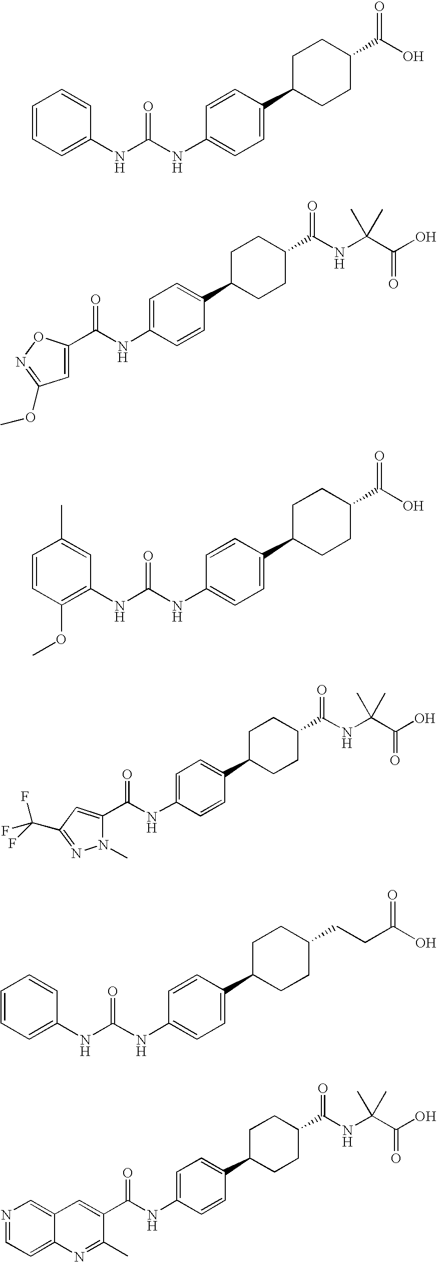 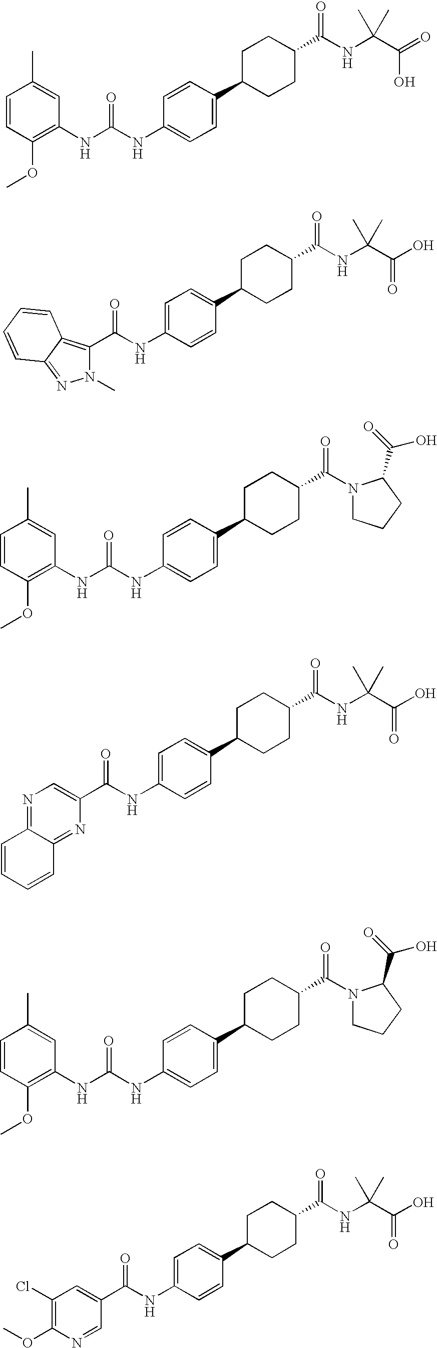 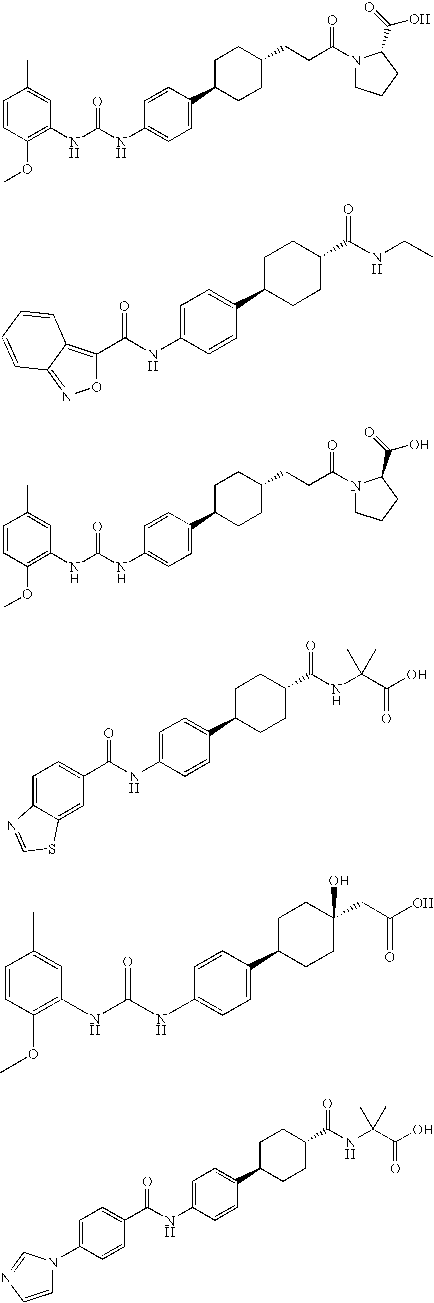 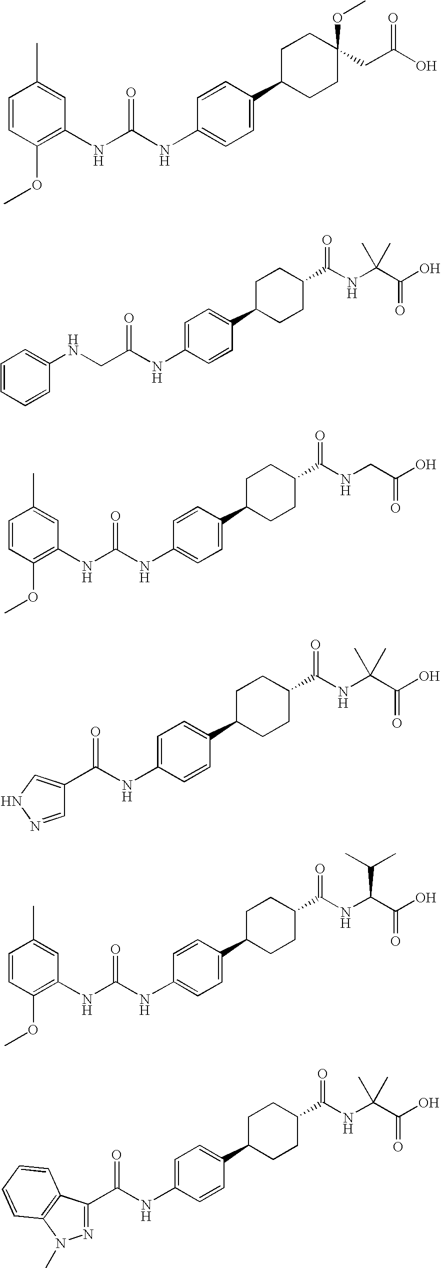 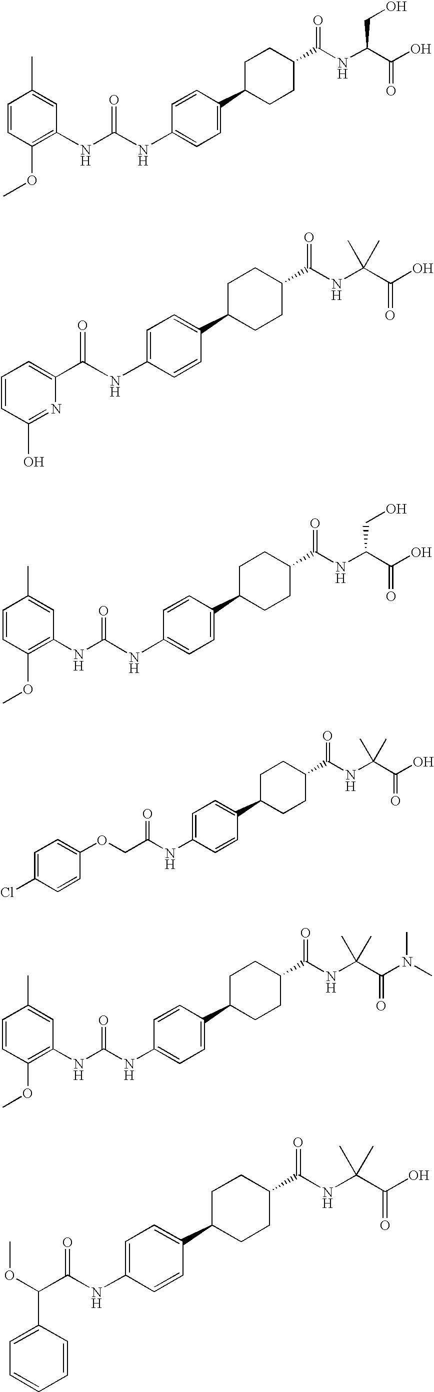 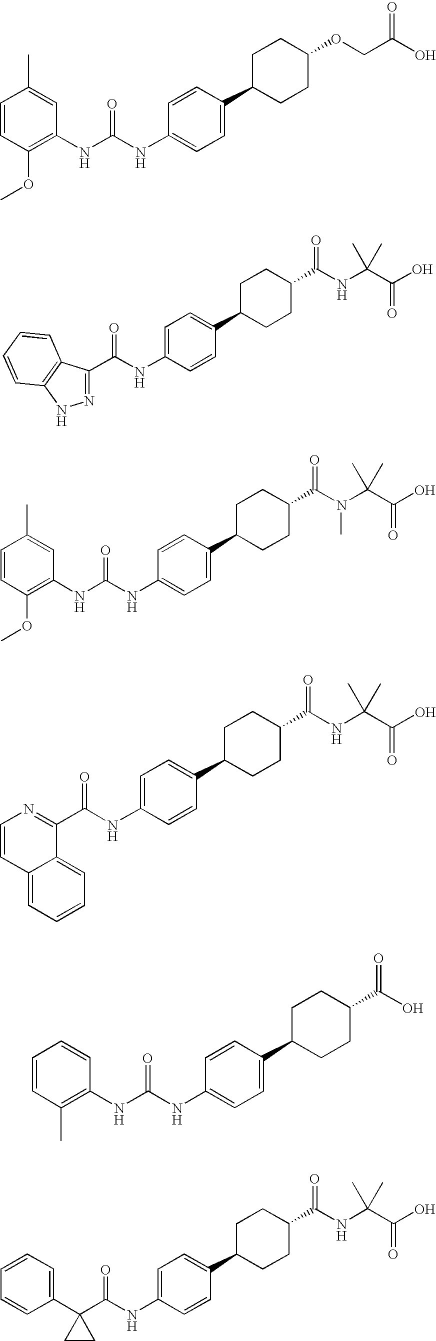 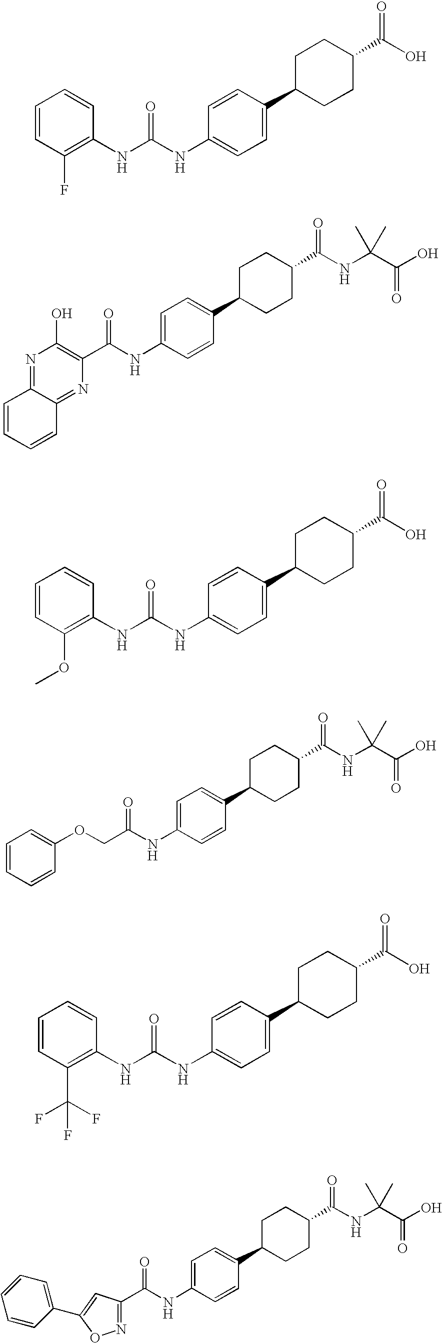 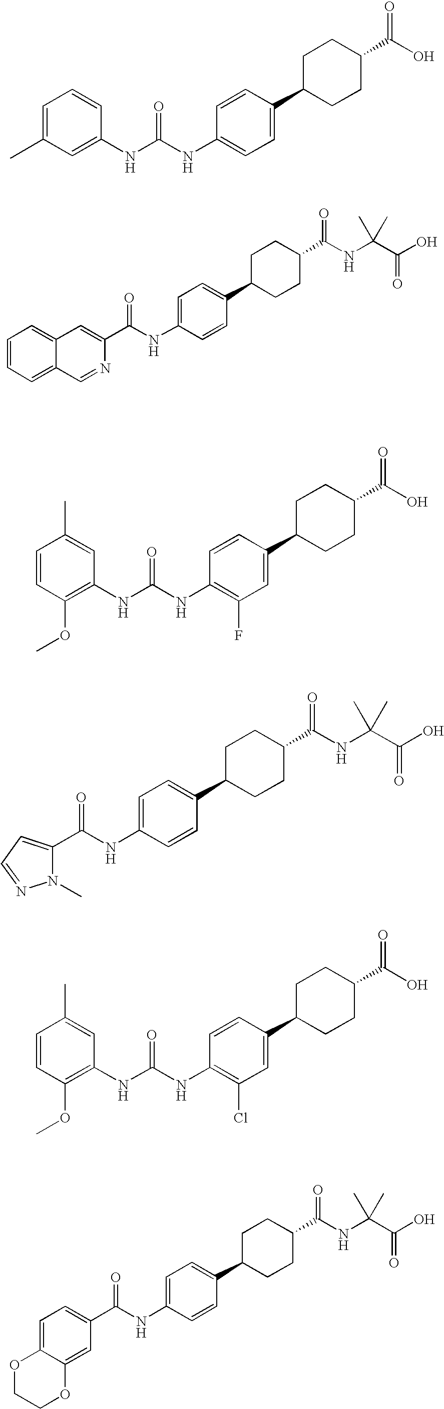 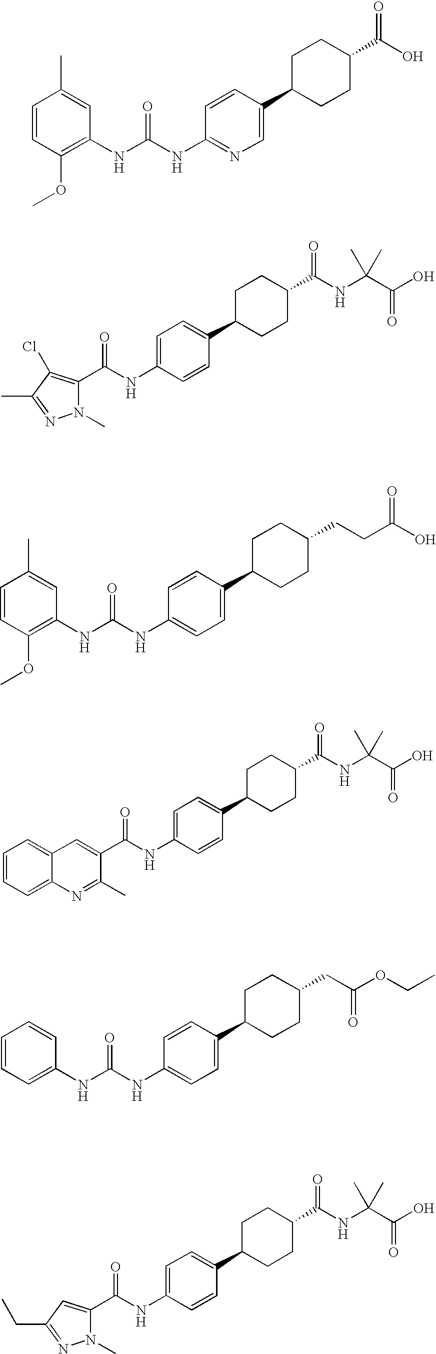 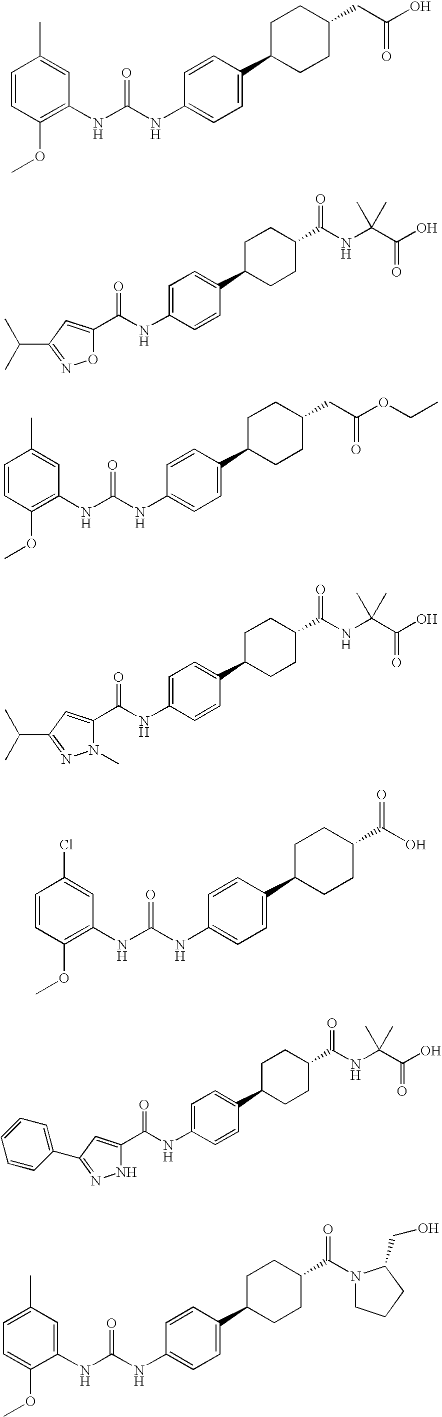 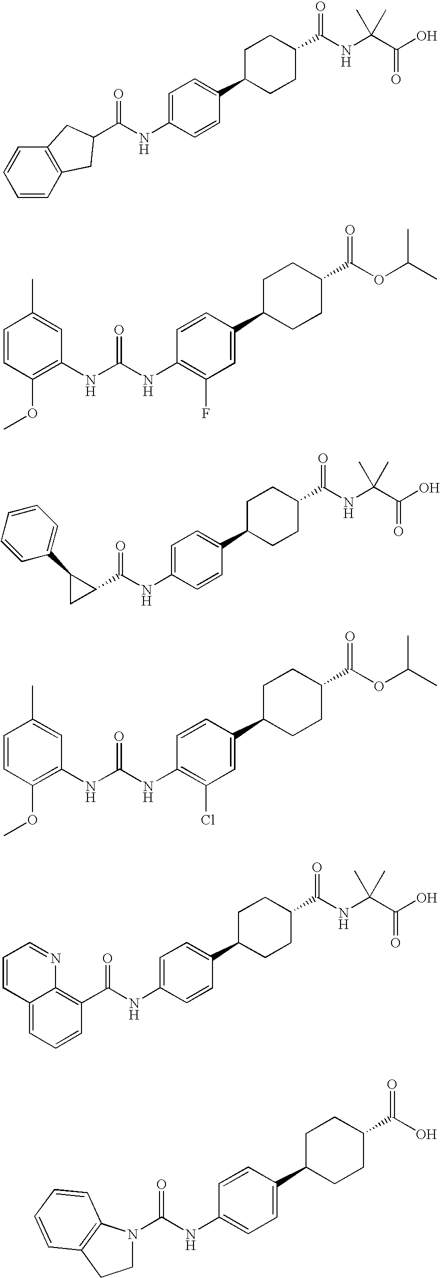 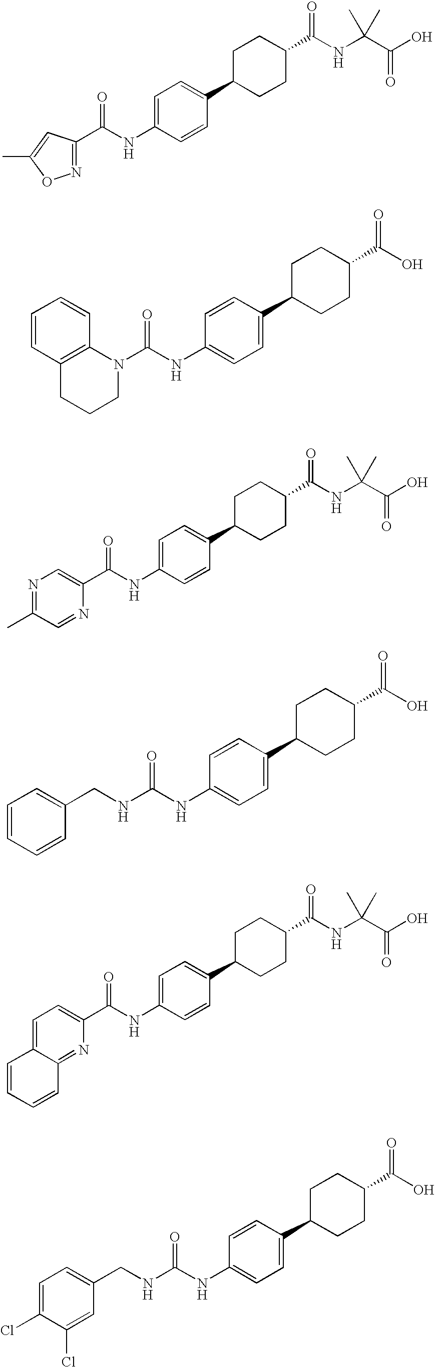 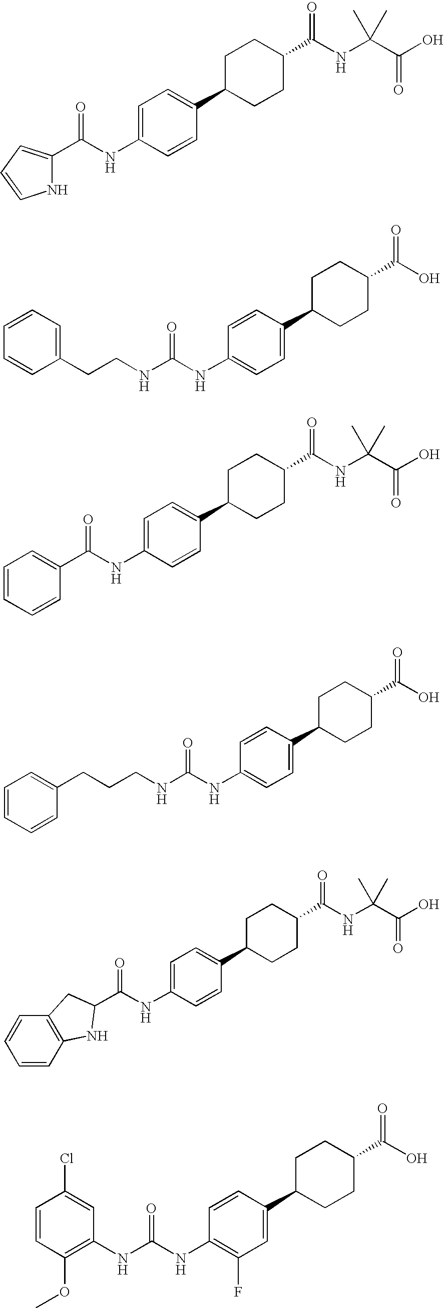 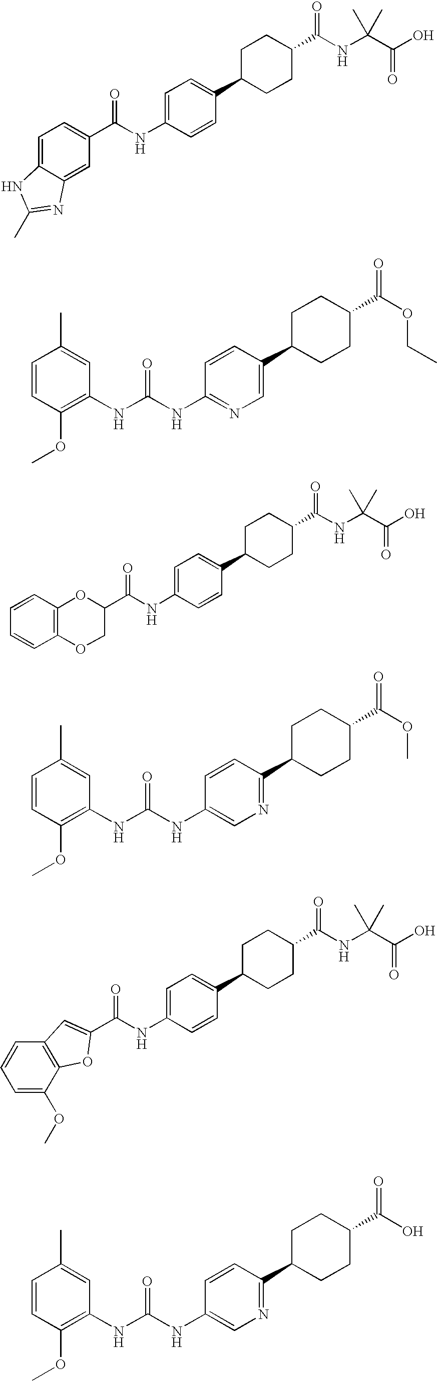 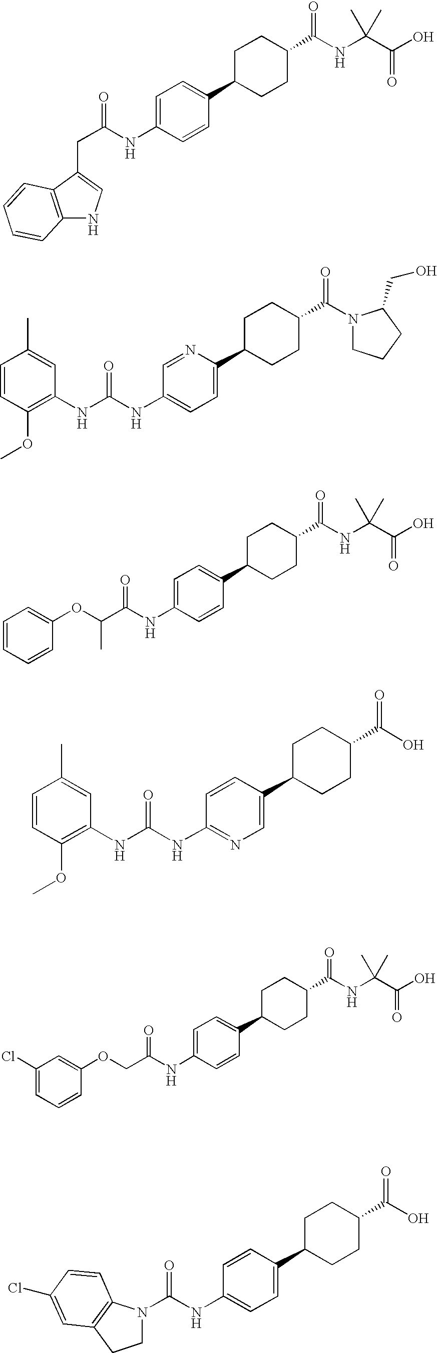 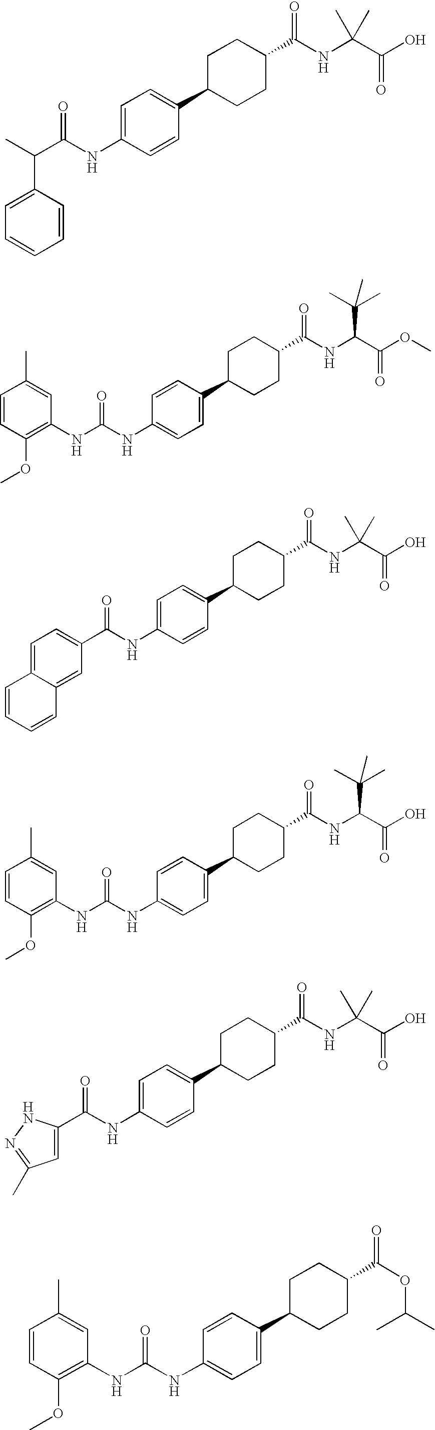 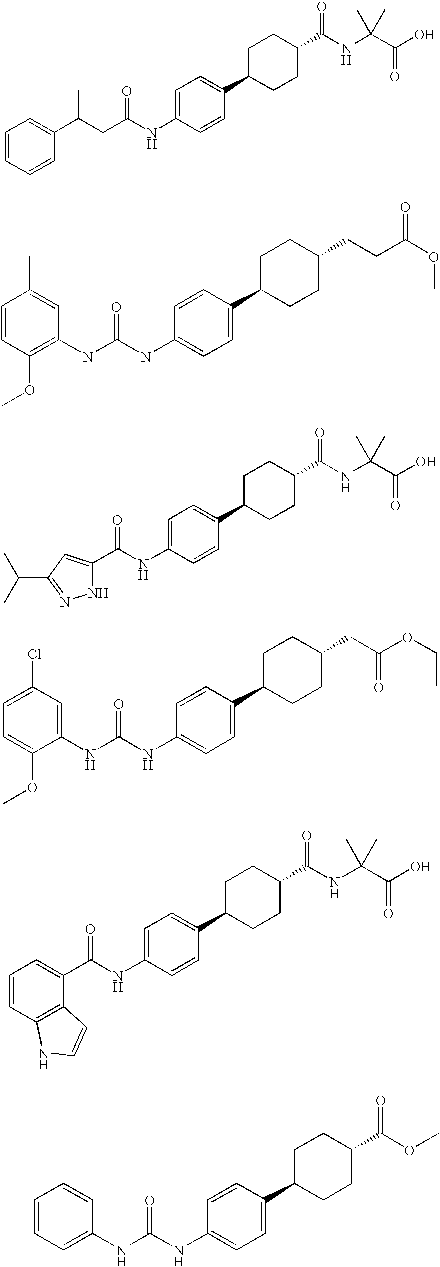 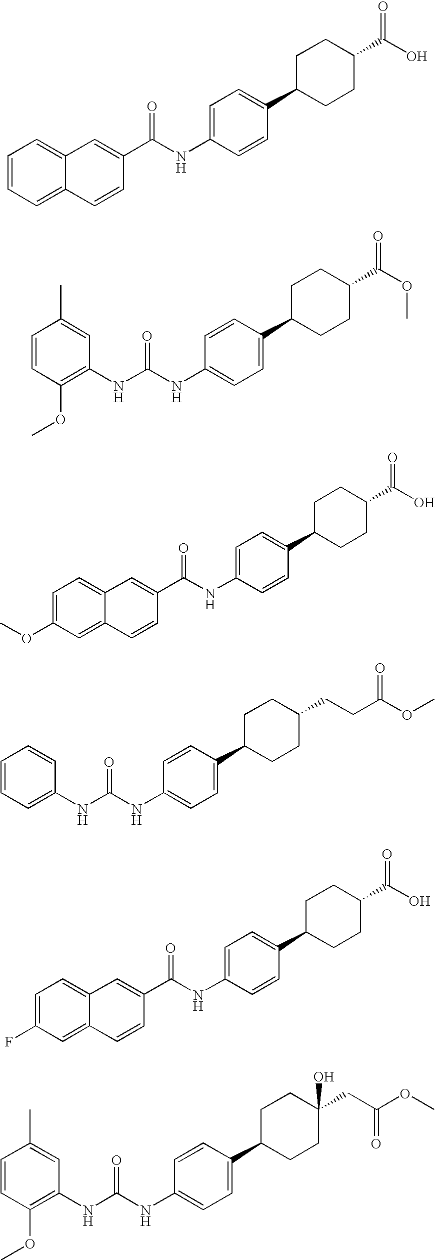 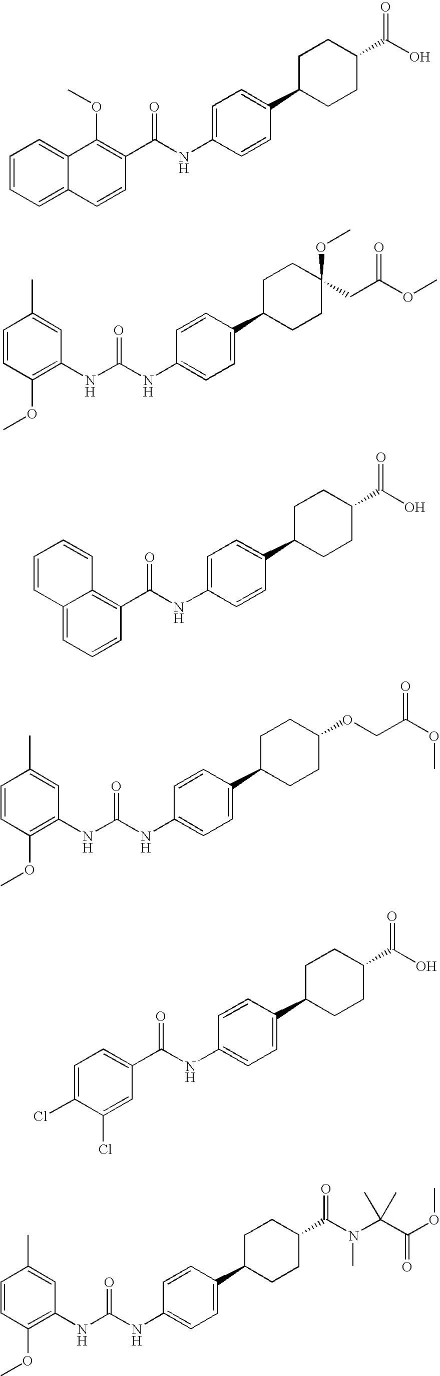 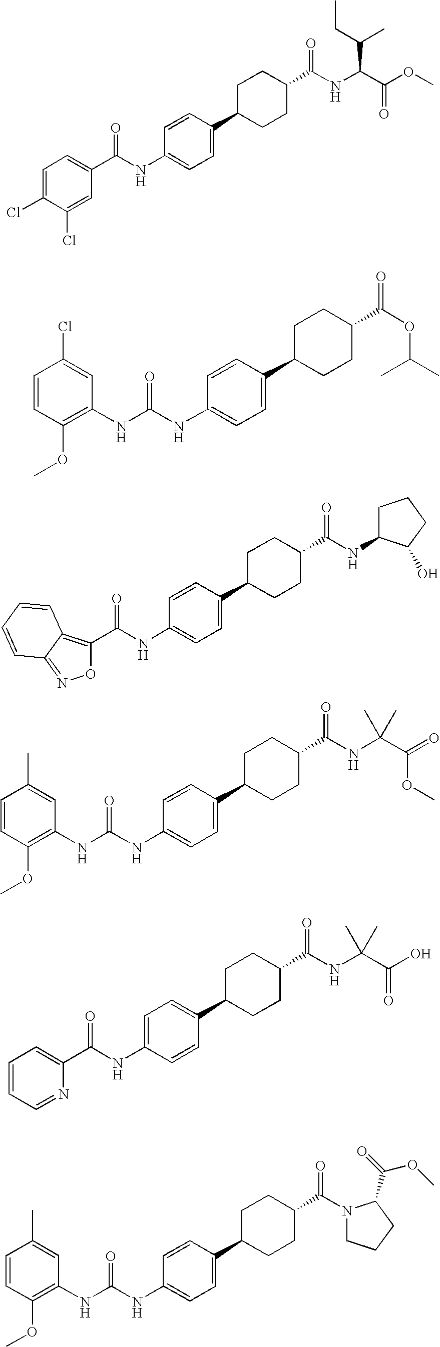 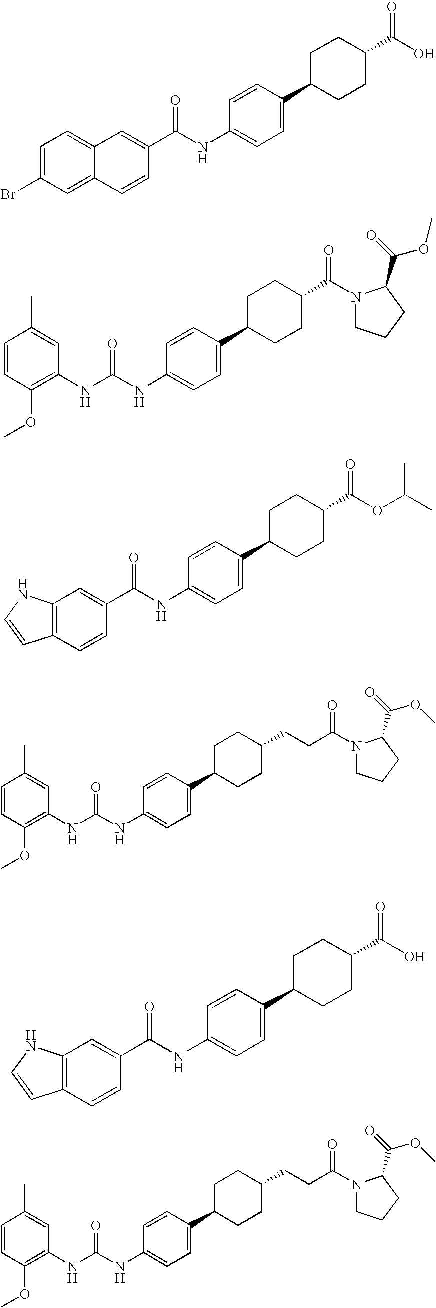 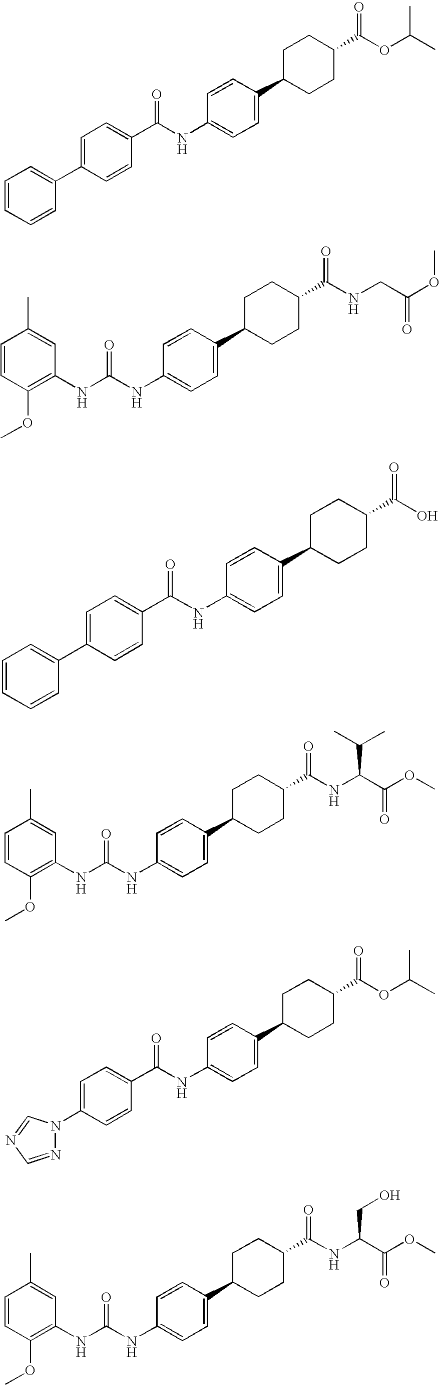 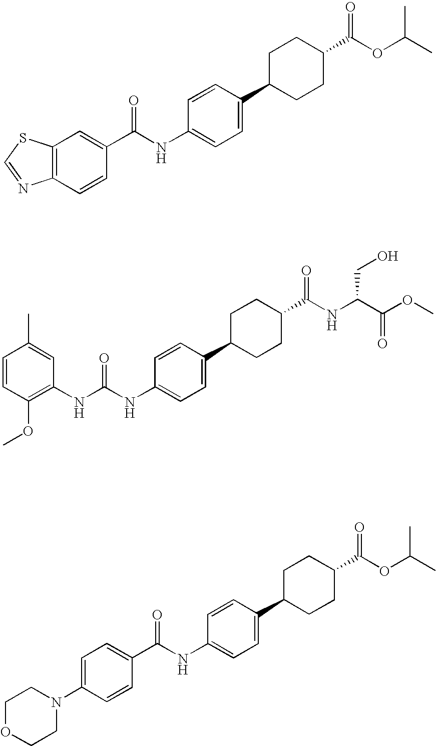
or a pharmaceutically acceptable salt or ester thereof.
